(19) United States
(12) Patent Application Publication
(10) Pub. No.: US 2014/0007296 A1
(43) Pub. Date: Jan. 2, 2014

(54) Methods and Compositions Using Small Interfering RNA (SIRNA) for Nematode Control in Plants

(71) Applicants: Xiang Huang, Durham, NC (US); Thomas Z. McNeill, Frisco, TX (US); Michael Schweiner, Durham, NC (US)

(72) Inventors: Xiang Huang, Durham, NC (US); Thomas Z. McNeill, Frisco, TX (US); Michael Schweiner, Durham, NC (US)

(73) Assignee: SYNGENTA PARTICIPATIONS AG, Basel (CH)

(21) Appl. No.: 13/992,659

(22) PCT Filed: Dec. 9, 2011
(86) PCT No.: PCT/US2011/064082

§ 371 Date: Sep. 16, 2013

Related U.S. Application Data
(60) Provisional application No. 61/421,275, filed on Dec. 9, 2010.

(51) Int. Cl. C12N 15/113

(52) U.S. Cl. CPC C12N 15/113
USPC 800/279; 435/468; 435/415; 435/419; 435/320.1; 514/44 A; 536/24.5; 800/301

(57) ABSTRACT

The present invention provides a double stranded RNA molecule comprising an antisense strand and a sense strand, wherein the nucleotide sequence of the antisense strand is complementary to a portion of the nucleotide sequence of a Hg-rps-23 gene of a soybean cyst nematode, nucleic acid molecules encoding the RNA molecules and compositions comprising the nucleic acid molecules and RNA molecules of this invention, as well as methods of their use in enhancing resistance of a plant or plant cell to nematode infestation and infection.

### STATEMENT OF PRIORITY

This application claims the benefit, under 35 U.S.C. §119(e) of U.S. Provisional Application Ser. No. 61/421,275, filed Dec. 9, 2010, the entire contents of which are incorporated by reference herein.

### FIELD OF THE INVENTION

The invention relates to the control of nematode parasitism in plants using small interfering RNA (siRNA).

### BACKGROUND OF THE INVENTION

Plant parasites (pests and pathogens) cause billion dollar crop losses world-wide each year. The nematode, in particular, the soybean cyst nematode (SCN), is the number one pathogen of soybean.

Nematodes are obligate, sedentary endoparasites that feed on the roots, leaves and stems of more than 2,000 vegetables, fruits, and ornamental plants, causing an estimated $100 billion crop loss worldwide.

Nematodes are present throughout the United States, but are mostly a problem in warm, humid areas of the south and west, as well as in sandy soils. Soybean cyst nematode (SCN), Heterodera glycines, was first discovered in North Carolina in 1954. It is the most serious pest of soybean plants. Once SCN is present in a field, it cannot feasibly be eradicated using known methods. Although soybean is the major economic crop attacked by SCN, SCN parasitizes some fifty hosts in total, including field crops, vegetables, ornamentals, and weeds.

Signs of nematode damage include stunting and yellowing of leaves, as well as wilting of the plants during hot periods. However, nematodes, including SCN, can cause significant yield loss without obvious above-ground symptoms. SCN infection in a plant can 1) result in dwarfed or stunted roots, 2) decrease the number of nitrogen-fixing nodules on the roots, and 3) make the roots more susceptible to attack by other soil-borne plant pathogens.

SCN has a life cycle consisting of an egg stage, four juvenile stages and an adult stage. After the first molt within the egg, SCN second stage juveniles (J2) hatch, move through the soil, penetrate roots and move toward the vascular cylinder. J2 is the only life stage of the nematode that can infect soybean roots. Migratory juveniles select a host cell in the cortex, endodermis, or pericycle and induce host cell fusion as part of the formation of a permanent feeding site called a syncytium. At this point the nematode becomes sedentary and differentiates to the third (J3) and fourth (J4) juvenile stages and then matures to an adult female or male. The actively feeding nematodes thus steal essential nutrients from the plant resulting in yield loss. As the nematodes feed, they swell and eventually the female nematodes become so large that they break through the root tissue and are exposed on the surface of the root.

Male nematodes, which are not swollen as adults, undergo a metamorphosis to resume a vermiform shape at the J4 stage and migrate back out of the root to fertilize adult females. The males then die, while the females remain attached to the root system and continue to feed. Following fertilization, the female produces eggs, most of which remain inside the body. After dying, the female body develops into a hardened cyst that encases the eggs. Cysts eventually dislodge and are found free in the soil. The walls of the cyst become very tough, providing protection for the 200-400 eggs contained within. SCN eggs survive within the cyst until proper hatching conditions occur. Although many of the eggs may hatch within the first year, many will survive within the cysts for several years.

Traditional practices for managing SCN include maintaining proper fertility and soil pH levels in SCN-infested land; controlling other plant diseases, as well as insect and weed pests; using sanitation practices such as plowing, planting, and cultivating of SCN-infested fields only after working non-infested fields; cleaning equipment thoroughly after working in infested fields; not using seed from plants grown on infested land for planting non-infested fields unless the seed has been properly cleaned; rotating infested fields and alternating host crops with non-host crops, such as, corn, oat and alfalfa; using pesticides or fumigants (e.g., nematicides); and planting resistant soybean varieties. While many of these can be effective, in addition to being time consuming and costly to implement, some of these approaches are no longer feasible, such as the application of nematicides, due to their toxicity and negative environmental impact. Thus, there is currently no efficient and effective approach to control of nematode infection in plants. Therefore, there is a need for compositions and methods for preventing, controlling, and reducing nematode parasitism in plants.

Accordingly, the present invention overcomes the deficiencies in the art by providing compositions and methods comprising small interfering RNAs for control of nematode infestation, infection and disease in plants.

### SUMMARY OF THE INVENTION

The present invention provides a double stranded RNA molecule comprising an antisense strand and a sense strand, wherein the nucleotide sequence of the antisense strand is complementary to a portion of the nucleotide sequence of a Hg-rps-23 gene of a soybean cyst nematode, the portion consisting essentially of about 18 to about 25 consecutive nucleotides of SEQ ID NO:931 (481 nt sequence of Hg-rps-23); wherein the double stranded RNA molecule inhibits expression of the Hg-rps-23 gene.

In addition, the present invention provides a chimeric nucleic acid molecule comprising an antisense strand having the nucleotide sequence of any of SEQ ID NOs:464-926 operably associated with a plant microRNA precursor molecule.

Also provided herein is an artificial plant microRNA precursor molecule comprising an antisense strand having the nucleotide sequence of any of SEQ ID Nos:464-926.

Furthermore, the present invention provides a composition comprising two or more of the RNA molecules of this invention wherein the two or more RNA molecules each comprise a different antisense strand.

A composition is also provided, comprising two or more of the chimeric nucleic acid molecules of this invention, wherein the two or more chimeric nucleic acid molecules each comprise a different antisense strand, as well as a composition comprising two or more of the artificial plant microRNA precursor molecules of this invention, wherein the two or more artificial plant microRNA precursor molecules each comprise a different antisense strand.

The present invention also provides a transformed plant cell comprising a nucleic acid molecule, a nucleic acid construct, a chimeric nucleic acid molecule, an artificial plant microRNA precursor molecule and/or a composition of this invention, wherein the transformed plant cell has enhanced resistance to soybean cyst nematode infection as compared to a control plant cell.

Furthermore, the present invention provides a transgenic plant comprising a nucleic acid molecule, a nucleic acid construct, a chimeric nucleic acid molecule, an artificial plant microRNA precursor molecule and/or a composition of this invention, wherein the transgenic plant has enhanced resistance to soybean cyst nematode infection as compared to a control plant.

It is further contemplated that a nucleic acid molecule, a nucleic acid construct, a chimeric nucleic acid molecule, an artificial plant microRNA precursor molecule and/or a composition of this invention can be employed in various methods. Thus, the present invention additionally provides a method of enhancing resistance of a plant cell to infection by a nematode, comprising introducing into the plant cell a nucleic acid molecule, a nucleic acid construct, a chimeric nucleic acid molecule, an artificial plant microRNA precursor molecule and/or a composition of this invention, thereby enhancing resistance of the plant cell to infection by the nematode.

Also provided herein is a method for controlling the infection of a plant cell by a nematode, comprising contacting the nematode infecting the plant cell with a nucleic acid molecule, a nucleic acid construct, a chimeric nucleic acid molecule, an artificial plant microRNA precursor molecule and/or a composition of any of this invention, thereby controlling infection of the plant cell by the nematode.

Additional embodiments include a method of enhancing resistance of a plant to infection by a nematode, comprising introducing into cells of the plant a nucleic acid molecule, a nucleic acid construct, a chimeric nucleic acid molecule, an artificial plant microRNA precursor molecule and/or a composition of this invention, thereby enhancing resistance of the plant to infection by the nematode.

The present invention also provides a method for controlling the infection of a plant by a nematode, comprising contacting the nematode infecting the plant with a nucleic acid molecule, a nucleic acid construct, a chimeric nucleic acid molecule, an artificial plant microRNA precursor molecule and/or a composition of this invention, thereby controlling infection of the plant by the nematode.

Further aspects of this invention include a method of reducing nematode cyst development on roots of a plant infected by a nematode, comprising introducing into cells of the plant a nucleic acid molecule, a nucleic acid construct, a chimeric nucleic acid molecule, an artificial plant microRNA precursor molecule and/or a composition of this invention, thereby reducing nematode cyst development on roots of the plant.

Additionally provided herein is a method of producing a transformed plant cell having enhanced resistance to nematode infection, comprising introducing into the plant cell a nucleic acid molecule, a nucleic acid construct, a chimeric nucleic acid molecule, an artificial plant microRNA precursor molecule and/or a composition of this invention, thereby producing a transformed plant cell having enhanced resistance to nematode infection relative to a control plant cell.

Furthermore, the present invention provides a method of producing a transgenic plant having enhanced resistance to nematode infection, comprising transforming cells of the plant with a nucleic acid molecule, a nucleic acid construct, a chimeric nucleic acid molecule, an artificial plant microRNA precursor molecule and/or a composition of this invention, thereby producing a transgenic plant having enhanced resistance to nematode infection relative to a control plant.

An additional embodiment includes a method of making a transgenic plant having enhanced resistance to nematode infection, comprising: a) transforming a plant cell with a nucleic acid molecule, a nucleic acid construct, a chimeric nucleic acid molecule, an artificial plant microRNA precursor molecule and/or a composition of this invention to produce a transformed plant cell; and b) growing the transformed plant cell into a transgenic plant, whereby the transgenic plant has enhanced resistant to nematode infection relative to a control plant.

In yet further embodiments, the present invention provides a crop comprising a plurality of the transgenic plant of any of the respective preceding claims, planted together in an agricultural field, as well as a method of improving crop yield, comprising: a) introducing a nucleic acid molecule, a nucleic acid construct, a chimeric nucleic acid molecule, an artificial plant microRNA precursor molecule and/or a composition of this invention into cells of a plant; and b) cultivating a plurality of the plant of (a) as a crop, resulting in a plurality of plants having enhanced resistance to nematode infection, thereby improving crop yield. These and other aspects of the invention are set forth in more detail in the description of the invention below.

### BRIEF DESCRIPTION OF THE DRAWINGS

FIG. 1. Photographs of J2s after each treatment.

FIG. 2. RNAi soaking and reproduction assay on soybean (Error bar=standard error).

FIG. 3. Effect of in-planta shRNA on SCN development (Error bar=standard error).

FIG. 4. amiR-rps23 hairy root-SCN assay (n=events; error bar=standard error).

FIG. 5. Northern blot to detect si-rps23-1 small RNA. Si-rps23-1 (arrows) was generated in hairy root samples (lanes 3, 4, 5). Lane 2=negative control roots. Lane 1=molecular marker.

FIGS. 6A-E. Effects of sh-rps23-1 on SCN cyst formation in transgenic whole plants. The average cysts of homozygous plants of the same events are reduced compared to either the null or heterozygous plants. A. Event SYNR092608A003A; B. Event SYNR093000A003A; C. Event SYNR093002A002A; D. Event SYNR093008A004A; E. SYNR093000A007A.

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety

The present invention is based on the unexpected discovery that small interfering RNAs can be used to control nematode infection in a plant and impart to a plant enhanced resistance to nematode infestation and/or infection. Thus, in one aspect, the present invention provides a double stranded RNA molecule comprising an antisense strand and a sense strand, wherein the nucleotide sequence of the antisense strand is complementary to a portion of the nucleotide sequence of a Hg-rps-23 gene of a soybean cyst nematode, the portion consisting essentially of about 18 to about 25 consecutive nucleotides of SEQ ID NO:931; wherein the double stranded RNA molecule inhibits expression of the Hg-rps-23 gene. The double stranded RNA molecule can comprise, consist essentially of or consist of about 18 to about 25 nucleotides (e.g., 18, 19, 20, 21, 22, 23, 24, or 25). Additional nucleotides can be added at the 3′ end, the 5′ end or both the 3′ and 5′ ends to facilitate manipulation of the RNA molecule but that do not materially affect the basic characteristics or function of the double stranded RNA molecule in RNA interference (RNAi).

In some embodiments, the RNA molecule of this invention is designed to target a portion of the nucleotide sequence of the Hg-rps-23 gene consisting essentially of the nucleotide sequence of any of SEQ ID NOs:1-463 (Table 1). Nonlimiting examples of an RNA molecule of this invention include an RNA molecule that targets the portion of the nucleotide sequence of the Hg-rps-23 gene consisting essentially of the nucleotide sequence of SEQ ID NO:64 and an RNA molecule that targets the portion of the nucleotide sequence of the Hg-rps-23 gene consists essentially of the nucleotide sequence of SEQ ID NO:258.

Thus, in various embodiments of the double stranded RNA molecule of this invention, the nucleotide sequence of the antisense strand can consist essentially of the nucleotide sequence of any of SEQ ID NOs:464-926 (Table 2) and in particular nonlimiting examples, the nucleotide sequence of the antisense strand can consist essentially of the nucleotide sequence of SEQ ID NO:863 or the nucleotide sequence of the antisense strand can consist essentially of the nucleotide sequence of SEQ ID NO:669. It is to be understood that the nucleotide sequences of SEQ ID NOs:464-926 (Table 2), including SEQ ID NO:863 and SEQ ID NO:669, which are all 19 nucleotides in length, can have one nucleotide at either the 3′ or 5′ end deleted or can have up to 6 nucleotides added at the 3′ end, the 5′ end or both, in any combination to achieve an antisense strand consisting essentially of the nucleotide sequence of any of SEQ ID NOs: 464-926 (Table 2), as it would be understood that the deletion of the one nucleotide or the addition of up to the six nucleotides do not materially affect the basic characteristics or function of the double stranded RNA molecule identified as any of SEQ ID NOs:464-926 (Table 2). Such additional nucleotides can be nucleotides that extend the complementarity of the antisense strand along the target sequence and/or such nucleotides can be nucleotides that facilitate manipulation of the RNA molecule or a nucleic acid molecule encoding the RNA molecule, as would be known to one of ordinary skill in the art. For example, in the exemplary siRNA molecules provided herein, a TT overhang at the 3; end is present, which is used to stabilize the siRNA duplex and does not affect the specificity of the siRNA.

In some embodiments of this invention, the sense strand of the double stranded RNA molecule can be fully complementary to the antisense strand or the sense strand can be substantially complementary or partially complementary to the antisense strand. By substantially or partially complementary is meant that the sense strand and the antisense strand can be mismatched at about 1, 2, 3, 4, 5, 6, 7, 8, 9, or 10 nucleotide pairings. Such mismatches can be introduced into the sense strand sequence, e.g., near the 3′ end, to enhance processing of the double stranded RNA molecule by Dicer, to duplicate a pattern of mismatches in a siRNA molecule inserted into a chimeric nucleic acid molecule or artificial microRNA precursor molecule of this invention (see Examples section), and the like, as would be known to one of skill in the art. Such modification will weaken the base pairing at one end of the duplex and generate strand asymmetry, therefore enhancing the chance of the antisense strand, instead of the sense strand, being processed and silencing the intended gene (Geng and Ding “Double-mismatched siRNAs enhance selective gene silencing of a mutant ALS-causing Allelel” Acta Pharmacol. Sin. 29:211-216 (2008); Schwarz et al. “Asymmetry in the assembly of the RNAi enzyme complex” Cell 115:199-208 (2003)). Nonlimiting examples of antisense/sense strand pairs in which mismatches have been introduced into the sense sequence include the sense strand AUUGCAAAUUGUUUUGAAATT (SEQ ID NO:928 with 3′ TT included; Table 3) and the corresponding antisense strand UUUCAGAGCAAUUUGCAAUTT (SEQ ID NO:836 with 3′ TT included) for si-rps23-2 and the sense strand UUGCAUCCUUGGUGAUUAATT (SEQ ID NO:929 with 3′TT included; Table 3), and the corresponding antisense strand UUGGUCGCCAAGGAUGCAATT (SEQ ID NO:740 with 3′ TT included) for si-rps23-3.

The present invention also includes embodiments in which the double stranded RNA molecule can be a short hairpin RNA (shRNA) molecule. Nonlimiting examples of nucleotide sequences encoding a shRNA of this invention include gaagcgcaatttccgagaatatcaagagtattctcggaaattgcgcttctgtttttt (SEQ ID NO:932), which is the shRNA sequence for sh-rps23-1, and acctgaagaagttgaacaatatcaagagtattgttcaacttcttcaggttgttttttt (SEQ ID NO:933), which is the shRNA sequence for sh-rps23-4. The design and production of any such shRNA of this invention is well known in the art.

In some embodiments of this invention, a chimeric nucleic acid molecule is provided, comprising an antisense strand having the nucleotide sequence of any of SEQ ID NOs:464-926 (Table 2) operably associated with a plant microRNA precursor molecule, which in some embodiments can be a soybean microRNA precursor molecule and in particular embodiments can be gma-MIR164.

In further embodiments, the present invention provides an artificial plant microRNA precursor molecule comprising an antisense strand having the nucleotide sequence of any of SEQ ID Nos:464-926 (Table 2), which in some embodiments can be a soybean microRNA precursor molecule and in particular embodiments can be gma-MIR164.

The use of artificial plant microRNAs to deliver a nucleotide sequence of interest (e.g., an artificial miRNA; siRNA/siRNA*) into a plant is well known in the art (see, e.g., Schwab et al. “Highly specific gene silencing by artificial microRNAs in Arabidopsis” The Plant Cell 18:1121-1133 (2006) and Examples section herein). In the present invention, such artificial plant microRNAs are chimeric or hybrid molecules, having a plant microRNA precursor backbone and a nematode (i.e., animal) siRNA sequence inserted therein. As would be understood by one of skill in the art, it is typically desirable to maintain mismatches that normally occur in the plant microRNA precursor sequence in any nucleotide sequence that is substituted into the plant microRNA precursor backbone. For example, to produce the artificial microRNA precursor molecule designated amiRrps23-1 described herein, the mismatch positions on the miR164/miR164* duplex were maintained in the si-rps-231si-rps-23-1* sequence (see Example section), resulting in the following sequence: ggatccagctccttgtttctcggaaattgcgcttcttagtctcttggatctcaaatgccactgaacccaagaagcgcaacctccgagaaca acacgggtttgagctc (SEQ ID NO:934).

Any plant microRNA (miRNA) precursor is suitable for the compositions and methods of this invention. Nonlimiting examples include any family members of the following plant miRNA precursors: miR156, miR159, miR160, miR161, miR162, miR163, miR164, miR165, miR166, miR167, miR168, miR169, miR170, miR171, miR172, miR173, miR319, miR390, miR393, miR395, miR396, miR397, miR398, miR399, miR408, miR447, as well as any other plant miRNA precursors now known or later identified.

Further provided herein is a nucleic acid construct (e.g., a vector or plasmid) comprising a nucleotide sequence encoding a double stranded nucleic acid molecule, a chimeric nucleic acid molecule and/or a plant microRNA precursor molecule of this invention.

The present invention further provides a composition comprising two or more of the RNA molecules of this invention, wherein the two or more RNA molecules each comprise a different antisense strand. The two or more RNA molecules can be present on the same nucleic acid construct, on different nucleic acid constructs or any combination thereof.

In particular embodiments, the double stranded nucleic acid molecule of this invention can comprise, consist essentially of or consist of an antisense strand consisting essentially of the nucleotide sequence of SEQ ID NO:863 (si-rps23-1 antisense) and/or an antisense strand consisting essentially of the nucleotide sequence of SEQ ID NO:669 (si-rps23-4 antisense).

Further provided herein is a composition comprising two or more of the nucleic acid constructs of this invention, wherein the two or more nucleic acid constructs each comprise a different antisense strand.

In addition, the present invention provides a composition comprising two or more of the nucleic acid molecules of this invention, wherein the two or more nucleic acid molecules each encode a different antisense strand.

Further provided herein is a composition comprising two or more of the nucleic acid constructs of this invention that encode a nucleic acid molecule encoding an antisense strand, wherein the two or more nucleic acid constructs each comprise a nucleic acid molecule encoding a different antisense strand.

The present invention also provides a composition comprising two or more of the chimeric nucleic acid molecules of this invention, wherein the two or more chimeric nucleic acid molecules each comprise a different antisense strand.

In yet further embodiments, the present invention provides a composition comprising two or more of the artificial plant microRNA precursor molecules of this invention, wherein the two or more artificial plant microRNA precursor molecules each comprise a different antisense strand.

It is understood that the compositions of this invention can comprise, consist essentially of or consist of any of the nucleic acid molecules, nucleic acid constructs, chimeric nucleic acid molecules and/or artificial microRNA precursor molecules in any combination and in any ratio relative to one another. Furthermore, by “two or more” is meant 2, 3, 4, 5, 6, 7, 8, 9, 10, etc., up to a total number of nucleic acid molecules, nucleic acid constructs, chimeric nucleic acid molecules and/or artificial microRNA precursor molecules of this invention.

The present invention encompasses plant cells and plants in accordance with the embodiments of this invention, Thus, in some embodiments, the present invention provides a transformed plant cell comprising a nucleic acid molecule, a nucleic acid construct, a chimeric nucleic acid molecule, an artificial plant microRNA precursor molecule and/or a composition of this invention, wherein the transformed plant cell has enhanced resistance to soybean cyst nematode infection as compared to a control plant cell.

Also provided herein is a transgenic plant comprising a nucleic acid molecule, a nucleic acid construct, a chimeric nucleic acid molecule, an artificial plant microRNA precursor molecule and/or a composition of this invention, wherein the transgenic plant has enhanced resistance to soybean cyst nematode infection as compared to a control plant.

In some embodiments, the transformed plant cell of this invention can be a cell of a legume plant. Furthermore, the transgenic plant of this invention can be a legume plant. Nonlimiting examples of a legume plant of this invention include soybean (cultivated and wild), green bean, snap bean, dry bean, red bean, lima bean, mung bean, kidney bean and bush bean.

In further embodiments, the transformed plant cell of this invention can be a cell of any plant that can be a host plant for nematode (e.g., soybean cyst nematode) infection. The transgenic plant of this invention can be any plant that can be a host plant for nematode infection. Nonlimiting examples of such host plants include lespedeza, vetch (common, hairy or winter), lupine, clover (crimson, scarlet or alsike), sweetclover, birdsfoot trefoil, crownvetch, garden pea, cowpea, black-eyed pea, black locust, Bells of Ireland, common chickweed, mousear chickweed, mullein, sicklepod, Digitalis penstemon, pokeweed, purslane, bittercress, Rocky Mountain beeplant, spotted geranium, toadflax, winged pigweed, vetch (American, Carolina or wood), burclover, toothed medic, dalea, Canadian milkvetch, borage, canary bird flower, caraway, Chinese lantern plant, coralbell, cup-flower, delphinium, foxglove, geum, common horehound, poppy, sage, snapdragon, sweet basil, sweetpea, verbena, henbit, hop clovers, beggars weed, tick clover, corn cockle, hogpeanut, milkpea, maize, barley, canola, wheat, cotton, tobacco, sugarbeet, potato, tomato, cabbage, cucumber, lettuce and wildbean.

Various methods are provided herein, employing the nucleic acid molecules, nucleic acid constructs, chimeric nucleic acid molecules, artificial microRNA precursors and/or compositions of this invention. Thus, in one aspect, the present invention provides a method of enhancing resistance of a plant cell to infection by a nematode, comprising introducing into the cell a nucleic acid molecule, a nucleic acid construct, a chimeric nucleic acid molecule, an artificial plant microRNA precursor molecule and/or a composition of this invention, thereby enhancing resistance of the plant cell to infection by the nematode.

Also provided herein is a method for controlling the infection of a plant cell by a nematode, comprising contacting the nematode infecting the plant cell with a nucleic acid molecule, a nucleic acid construct, a chimeric nucleic acid molecule, an artificial plant microRNA precursor molecule and/or a composition of this invention, thereby controlling infection of the plant cell by the nematode.

In addition, the present invention provides a method of enhancing resistance of a plant to infection by a nematode, comprising introducing into cells of the plant a nucleic acid molecule, a nucleic acid construct, a chimeric nucleic acid molecule, an artificial plant microRNA precursor molecule and/or a composition of this invention, thereby enhancing resistance of the plant to infection by the nematode.

Further provided is a method for controlling the infection of a plant by a nematode, comprising contacting the nematode infecting the plant with a nucleic acid molecule, a nucleic acid construct, a chimeric nucleic acid molecule, an artificial plant microRNA precursor molecule and/or a composition of this invention, thereby controlling infection of the plant by the nematode.

Additional embodiments of this invention include a method of reducing nematode cyst development on roots of a plant infected by a nematode, comprising introducing into cells of the plant a nucleic acid molecule, a nucleic acid construct, a chimeric nucleic acid molecule, an artificial plant microRNA precursor molecule and/or a composition of this invention, thereby reducing nematode cyst development on roots of the plant.

Furthermore, the present invention provides a method of producing a transformed plant cell having enhanced resistance to nematode infection, comprising introducing into the cell a nucleic acid molecule, a nucleic acid construct, a chimeric nucleic acid molecule, an artificial plant microRNA precursor molecule and/or a composition of this invention, thereby producing a transformed plant cell having enhanced resistance to nematode infection relative to a control plant cell. The present invention also provides a transformed plant cell produced by such method.

Additionally provided herein is a method of producing a transgenic plant having enhanced resistance to nematode infection, comprising transforming cells of the plant with the nucleic acid molecule, the nucleic acid construct, the chimeric nucleic acid molecule, the artificial plant microRNA precursor molecule and/or the composition of any of the respective preceding claims, thereby producing a transgenic plant having enhanced resistance to nematode infection relative to a control plant. Also provided is a transgenic plant produced by such method.

Further aspects of the invention include a method of making a transgenic plant having enhanced resistance to nematode infection, comprising: a) transforming a plant cell with the nucleic acid molecule, the nucleic acid construct, the chimeric nucleic acid molecule, the artificial plant microRNA precursor molecule and/or the composition of any of the respective preceding claims to produce a transformed plant cell; and b) growing the transformed plant cell into a transgenic plant, whereby the transgenic plant has enhanced resistant to nematode infection relative to a control plant. A transgenic plant produced by such method is also provided herein.

A nematode of this invention includes, but is not limited to soybean cyst nematode (Heterodera glycines), the root-knot nematode species (Meloidogyne spp.), other cyst nematode species (Heterodera spp.), the lesion nematode species (Pratylenchus spp.), the reniform nematode (Rotylenchulus reniformis), the burrowing nematode (Radopholus similis), the citrus nematode (Tylenchulus semipenetrans), lance nematodes (Hoplolaimus spp.), stunt nematodes (Tylenchorhynchus spp.), spiral nematodes (Helicotylenchus spp.), sting nematodes (Belonoluimus spp.) and ring nematodes (Criconema spp.)

In accordance with the invention, a parasitic nematode is contacted with a siRNA molecule of this invention, which specifically inhibits expression of a target gene that is essential for survival, metamorphosis, or reproduction of the nematode. Preferably, the parasitic nematode comes into contact with the siRNA after entering a plant in which the siRNA of this invention is present. In one embodiment, the siRNA is encoded by a nucleic acid construct (e.g., a vector), which has been transformed into an ancestor of the infected plant. The nucleic acid construct expressing the siRNA can be under the transcriptional control of a root specific promoter or a parasitic nematode feeding cell-specific promoter.

In particular embodiments, the present invention provides double stranded RNA containing a nucleotide sequence that is fully complementary to a portion of the target gene for inhibition. However, it is to be understood that 100% complementarity between the antisense strand of the double stranded RNA molecule and the target sequence is not required to practice the present invention. Thus, sequence variations that might be expected due to genetic mutation, strain polymorphism, or evolutionary divergence can be tolerated. RNA sequences with insertions, deletions, and single point mutations relative to the target sequence may also be effective for inhibition. Thus, sequence identity and complementarity can be optimized by sequence comparison and alignment algorithms known in the art (see Gribskov and Devereux, Sequence Analysis Primer, Stockton Press, 1991) and calculating the percent difference between the nucleotide sequences by, for example, the Smith-Waterman algorithm as implemented in the BESTFIT software program using default parameters (e.g., University of Wisconsin Genetic Computing Group). Greater than 90% complementarity, or even 100% complementarity, between the inhibitory RNA and the portion of the target gene is preferred. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60° C. hybridization for 12-16 hours; followed by washing).

The dsRNA of the invention may optionally comprise a single stranded overhang at either or both ends. The double-stranded structure may be formed by a single self-complementary RNA strand (i.e., forming a hairpin loop) or two complementary RNA strands. RNA duplex formation may be initiated either inside or outside the cell. When the dsRNA of the invention forms a hairpin loop, it may optionally comprise an intron and/or a nucleotide spacer, which is a stretch of nucleotides between the complementary RNA strands, to stabilize the hairpin sequence in cells. The RNA may be introduced in an amount that allows delivery of at least one copy per cell. Higher doses of double-stranded material may yield more effective inhibition.

In some embodiments, the present invention provides a nucleic acid construct comprising a nucleic acid encoding a dsRNA molecule of this invention, wherein expression of the nucleic acid construct in a plant cell (e.g., a transformed plant cell) results in increased resistance to a nematode as compared to a wild-type variety of the plant cell (e.g., a control plant cell or nontransformed plant cell). As used herein, the term “nucleic acid construct” means a nucleic acid molecule capable of transporting another nucleic acid to which it is linked. One type of nucleic acid construct is a vector, which can be a transformation vector or an expression vector. Another type of nucleic acid construct of this invention is a “plasmid,” which refers to a circular double stranded nucleic acid loop into which additional nucleic acid segments can be ligated. Another type of nucleic acid construct is a viral vector, wherein additional nucleic acid segments can be ligated into a viral genome. Certain vectors are capable of autonomous replication in a plant cell into which they are introduced. Other vectors are integrated into the genome of a plant cell upon introduction into the plant cell, and are then replicated along with the plant cell genome. Moreover, certain vectors can direct the expression of genes or coding sequences to which they are operatively linked. Such vectors are referred to herein as “expression vectors.” In some embodiments of this invention, an expression vector can be a viral vector (e.g., potato virus X; tobacco rattle virus; Geminivirus).

An expression vector of the invention can comprise a nucleic acid of the invention in a form suitable for expression of the nucleic acid in a plant cell, which means that the expression vector includes one or more regulatory sequences, selected on the basis of the plant cells to be used for expression, which is operatively linked to the nucleic acid sequence to be expressed. With respect to an expression vector, “operatively linked” is intended to mean that the nucleotide sequence of interest is linked to the regulatory sequence(s) in a manner which allows for expression of the nucleotide sequence (e.g., in a plant cell when the vector is introduced into the plant cell). The term “regulatory sequence” is intended to include promoters, enhancers, and other expression control elements (e.g., polyadenylation signals) as are well known in the art. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of dsRNA desired, etc. The expression vectors of the invention can be introduced into plant cells to thereby produce dsRNA molecules encoded by nucleic acids as described herein.

In some embodiments of the present invention, the expression vector can comprise a regulatory sequence operably linked to a nucleotide sequence that is a template for one or both strands of the claimed dsRNA molecules. In one embodiment, the nucleic acid molecule further comprises a promoter flanking either end of the nucleic acid molecule, wherein the promoters drive expression of each individual DNA strand, thereby generating two RNAs that hybridize and form the dsRNA. In another embodiment, the nucleic acid molecule comprises a nucleotide sequence that is transcribed into both strands of the dsRNA on one transcription unit, wherein the sense strand is transcribed from the 5′ end of the transcription unit and the antisense strand is transcribed from the 3′ end, wherein the two strands are separated by about 3 to about 500 basepairs, and wherein after transcription, the RNA transcript folds on itself to form a hairpin. In accordance with the invention, the spacer region in the hairpin transcript can be any nucleic acid fragment.

In some embodiments of this invention, the introduced nucleic acid molecule may be maintained in the plant cell stably if it is incorporated into a non-chromosomal autonomous replicon or integrated into the plant chromosomes. Alternatively, the introduced nucleic acid molecule may be present on an extra-chromosomal non-replicating vector and be transiently expressed or transiently active. Whether present in an extra-chromosomal non-replicating vector or a vector that is integrated into a chromosome, the nucleic acid molecule can be present in a plant expression cassette. A plant expression cassette can contain regulatory sequences that drive gene expression in plant cells that are operably linked so that each sequence can fulfill its function, for example, termination of transcription by polyadenylation signals. Exemplary polyadenylation signals can be those originating from Agrobacterium tumefaciens t-DNA such as the gene known as octopine synthase of the Ti-plasmid pTiACH5 (Gielen et al. EMBO J. 3:835 (1984)) or functional equivalents thereof, but also all other terminators functionally active in plants are suitable. A plant expression cassette of this invention can also contain other operably linked sequences like translational enhancers such as the overdrive-sequence containing the 5′-untranslated leader sequence from tobacco mosaic virus enhancing the polypeptide per RNA ratio (Gallie et al. Nucl. Acids Research 15:8693-8711 (1987)).

A nucleic acid molecule of this invention can be introduced into a cell by any method known to those of skill in the art. In some embodiments of the present invention, transformation of a plant cell of this invention can comprise nuclear transformation. In other embodiments, transformation of a plant cell of this invention can comprises plastid transformation (e.g., chloroplast transformation).

Procedures for transforming plants are well known and routine in the art and are described throughout the literature. Non-limiting examples of methods for transformation of plants include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Mild et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Thus, in some embodiments, the introducing into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethyleneglycol-mediated transformation, any other electrical, chemical, physical and/or biological mechanism that results in the introduction of nucleic acid into the plant, plant part and/or cell thereof, or any combination thereof.

Agrobacterium-mediated transformation is a commonly used method for transforming plants, in particular, dicot plants, because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen & Willmitzer (1988) Nucleic Acids Res. 16:9877).

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of this invention. Alternatively, a cell or cells can be surrounded by the vector so that the vector is carried into the cell by the wake of the particle. Biologically active particles (e.g., dried yeast cells, dried bacterium or a bacteriophage, each containing one or more nucleic acids sought to be introduced) also can be propelled into plant tissue.

Thus, in particular embodiments of the present invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans at al. (Handbook of Plant Cell Cultures, Vol. 1, MacMillan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

Likewise, the genetic properties engineered into the transgenic seeds and plants, plant parts, and/or plant cells of the present invention described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A nucleotide sequence therefore can be introduced into the plant, plant part and/or plant cell in any number of ways that are well known in the art. The methods of the invention do not depend on a particular method for introducing one or more nucleotide sequences into a plant, only that they gain access to the interior of at least one cell of the plant.

Physical methods of introducing dsRNA into nematodes include injection of a solution containing the dsRNA or soaking the nematode in a solution of the dsRNA. Preferably, the dsRNA of the invention is introduced into nematodes when the nematodes ingest transgenic plants containing nucleic acid constructs encoding the dsRNA.

Thus, in some embodiments, the present invention provides plants, plant parts and/or plant cells having enhanced or increased resistance to nematode infestation or infection, produced by the methods of the present invention. In further embodiments, the present invention provides plants, plant parts and/or plant cells having increased or enhanced resistance to soybean cyst nematode infestation or infection, produced by the methods of the present invention. In still other embodiments, the present invention provides soybean plants, soybean plant parts and/or soybean plant cells having increased or enhanced resistance to soybean cyst nematode infestation or infection, produced by the methods of the present invention.

Further aspects of the present invention provide plants, plant parts and/or plant cells having reduced formation of soybean cyst nematode cysts produced by the methods of the present invention. In still further aspects, the present invention provides soybean plants, soybean plant parts and/or soybean plant cells having reduced formation of soybean cyst nematode cysts produced by the methods of the present invention.

In yet further aspects, the present invention provides a crop comprising a plurality of any transgenic plant of this invention, planted together in an agricultural field. In particular embodiments, the crop can be a legume crop and in certain embodiments the crop can be a soybean crop.

Also provided herein is a method of improving crop yield, comprising: a) introducing the nucleic acid molecule, the nucleic acid construct, the chimeric nucleic acid molecule, the artificial plant microRNA precursor molecule and/or the composition of any of the respective preceding claims into cells of a plant; b) cultivating a plurality of the plant of (a) as a crop, resulting in a plurality of plants having enhanced resistance to nematode infection, thereby improving crop yield.

### DEFINITIONS

As used herein, “a,” “an” or “the” can mean one or more than one. For example, a cell can mean a single cell or a multiplicity of cells.

As used herein, “and/or” refers to and encompasses any and all possible combinations of one or more of the associated listed items, as well as the lack of combinations when interpreted in the alternative (or).

Further, the term “about,” as used herein when referring to a measurable value such as an amount of a compound or agent, dose, time, temperature, and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The term “plant” is intended to encompass plants at any stage of maturity or development, as well as any tissues or organs (plant parts) taken or derived from any such plant unless otherwise clearly indicated by context. The present invention also includes transgenic seeds produced by the transgenic plants of the present invention. In one embodiment, the seeds are true breeding for an increased resistance to nematode infection as compared to a wild-type variety of the plant seed. In particular embodiments of the invention, the plant is a soybean plant.

As used herein, the term “plant part” includes but is not limited to pollen, seeds, branches, fruit, kernels, ears, cobs, husks, stalks, root tips, anthers, stems, roots, flowers, ovules, stamens, leaves, embryos, meristematic regions, callus tissue, anther cultures, gametophytes, sporophytes, pollen, microspores, protoplasts, hairy root cultures, and the like. plant cells including plant cells that are intact in plants and/or parts of plants, plant protoplasts, plant tissues, plant cell tissue cultures, plant calli, plant clumps, and the like. Further, as used herein, “plant cell” refers to a structural and physiological unit of the plant, which comprises a cell wall and also may refer to a protoplast. Thus, as used herein, a “plant cell” includes, but is not limited to, a protoplast, gamete producing cell, and a cell that regenerates into a whole plant. Tissue culture of various tissues of plants and regeneration of plants therefrom is well known in the art.

A plant cell of the present invention can be in the form of an isolated single cell or can be a cultured cell or can be a part of a higher-organized unit such as, for example, a plant tissue or a plant organ.

As used herein, the term “enhanced resistance” or “increased resistance” refers to the reduction, delay and/or prevention of a nematode infestation and/or infection in a transformed plant cell and/or transgenic plant of this invention as compared with a nontransformed plant cell (e.g., control plant cell) or a nontransgenic plant (e.g., control plant). Reducing, delaying or preventing an infection by a nematode will cause a plant to have enhanced or increased resistance to the nematode, however, such increased resistance does not imply that the plant necessarily has 100% resistance to infestation or infection. In some embodiments, the resistance to infestation or infection by a nematode in a transformed plant cell or transgenic plant of this invention is greater than about 10%, 20%, 30%, 40%, 50%, 60%, 70%, 80%, 90%, or 95% in comparison to a wild type plant or plant cell (e.g., a control plant or control plant cell) that is not resistant to nematodes. The plant's resistance to infection by the nematode may be due to the death, sterility, arrest in development, and/or impaired mobility of the nematode upon exposure to the dsRNA specific to an essential gene.

The terms “reduce,” “reduced,” “reducing,” “reduction,” “diminish,” and “decrease” (and grammatical variations thereof), as used herein, describe a decrease in the soybean cyst nematode cyst formation on a plant (e.g., soybean) by the introduction of a nucleic acid molecule, nucleic acid construct, chimeric nucleic acid molecule, artificial microRNA precursor molecule and/or composition of the present invention into the plant, thereby producing a transgenic plant having decreased or reduced cyst formation on the transgenic plant. This decrease in cyst formation can be observed, by comparing the number of cysts formed on the plant transformed with the nucleic acid molecule, nucleic acid construct, chimeric nucleic acid molecule, artificial microRNA precursor molecule and/or composition to the number formed on a soybean plant that is not transformed with the nucleic acid molecule, nucleic acid construct, chimeric nucleic acid molecule, artificial microRNA precursor molecule and/or composition.

As used herein, the term “amount sufficient to inhibit expression” refers to a concentration or amount of the dsRNA that is sufficient to reduce levels or stability of mRNA or protein produced from a target gene (e.g., hg-rps-23) in a nematode (e.g., soybean cyst nematode). As used herein, “inhibiting expression” refers to the absence or observable decrease in the level of protein and/or mRNA product from a target gene. Inhibition of target gene expression may be lethal to the nematode, or such inhibition may delay or prevent entry into a particular developmental stage (e.g., metamorphosis), if plant disease is associated with a particular stage of the nematode's life cycle. The consequences of inhibition can be confirmed by examination of the outward properties of the nematode (e.g., as described in the Examples section here).

As used herein, “RNAi” or “RNA interference” refers to the process of sequence-specific post-transcriptional gene silencing (e.g., in nematodes), mediated by double-stranded RNA (dsRNA). As used herein, “dsRNA” refers to RNA that is partially or completely double stranded. Double stranded RNA is also referred to as small interfering RNA (siRNA), small interfering nucleic acid (siNA), microRNA (mRNA), and the like. In the RNAi process, dsRNA comprising a first (antisense) strand that is complementary to a portion of a target gene and a second (sense) strand that is fully or partially complementary to the first antisense strand is introduced into an organism (e.g., nematode), by, e.g., soaking and/or feeding. After introduction into the organism, the target gene-specific dsRNA is processed into relatively small fragments (siRNAs) and can subsequently become distributed throughout the organism, leading to a loss-of-function mutation having a phenotype that, over the period of a generation, may come to closely resemble the phenotype arising from a complete or partial deletion of the target gene. Alternatively, the target gene-specific dsRNA is processed into relatively short fragments by a plant cell containing the RNAi processing machinery; and when the plant-processed short dsRNA is ingested by a parasitic organism, such as a nematode, the loss-of-function phenotype is obtained.

MicroRNAs (miRNAs) are non-protein coding RNAs, generally of between about 18 to about 25 nucleotides in length (commonly about 20-24 nucleotides in length in plants). These miRNas direct cleavage in trans of target transcripts, negatively regulating the expression of genes involved in various regulation and development pathways (Bartel, Cell, 116:281-297 (2004); Zhang et al. Dev. Biol. 289:3-16 (2006)). As such, miRNAs have been shown to be involved in different aspects of plant growth and development as well as in signal transduction and protein degradation. In addition, small endogenous mRNAs including miRNAs may also be involved in biotic stress responses such as pathogen attack. Since the first miRNAs were discovered in plants (Reinhart et al. Genes Dev. 16:1616-1626 (2002), Park et al. Curr. Biol, 12:1484-1495 (2002)) many hundreds have been identified. Furthermore, many plant miRNAs have been shown to be highly conserved across very divergent taxa. (Floyd et al. Nature 428:485-486 (2004); Zhang et al. Plant J. 46:243-259 (2006)). Many microRNA genes (MIR genes) have been identified and made publicly available in a database (miRBase; microrna.sanger.ac.uk/sequences). miRNAs are also described in U.S. Patent Publications 2005/0120415 and 2005/144669A1, the entire contents of which are incorporated by reference herein.

Genes encoding miRNAs yield primary miRNAs (termed a “pri-miRNA”) of 70 to 300 by in length that can form imperfect stem-loop structures. A single pri-miRNA may contain from one to several miRNA precursors. In animals, pri-miRNAs are processed in the nucleus into shorter hairpin RNAs of about 65 nt (pre-miRNAs) by the RNaseIII enzyme Drosha and its cofactor DGCR8/Pasha. The pre-miRNA is then exported to the cytoplasm, where it is further processed by another RNaseIII enzyme, Dicer, releasing a miRNA/miRNA* duplex of about 22 nt in size. In contrast to animals, in plants, the processing of pri-miRNAs into mature miRNAs occurs entirely in the nucleus using a single RNaseIII enzyme, DCL1 (Dicer-like 1). (Zhu. Proc. Natl. Acad. Sci. 105:9851-9852 (2008)). Many reviews on microRNA biogenesis and function are available, for example, see, Bartel Cell 116:281-297 (2004), Murchison et al. Curr. Opin. Cell Biol. 16:223-229 (2004), Dugas et al. Curr. Opin. Plant Biol. 7:512-520 (2004) and Kim Nature Rev. Mol. Cell. Biol. 6:376-385 (2005).

The term “plant microRNA precursor molecule” as used herein describes a small (˜70-300 nt) non-coding RNA sequence that is processed by plant enzymes to yield a ˜19-24 nucleotide product known as a mature microRNA sequence. The mature sequences have regulatory roles through complementarity to messenger RNA. The term “artificial plant microRNA precursor molecule” describes the non-coding miRNA precursor sequence prior to processing that is employed as a backbone sequence for the delivery of a siRNA molecule via substitution of the endogenous native miRNA/miRNA* duplex of the miRNA precursor molecule with that or a non-native, heterologous miRNA (amiRNA/amiRNA*; e.g., si-rps23-1/si-rps-23-1* or siRNA/siRNA*) that is then processed into the mature miRNA sequence with the siRNA sequence.

Also as used herein, the terms “nucleic acid,” “nucleic acid molecule,’ “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides. Nucleic acid sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, the terms “fragment” or “portion” when used in reference to a nucleic acid molecule or nucleotide sequence will be understood to mean a nucleic acid molecule or nucleotide sequence of reduced length relative to a reference nucleic acid molecule or nucleotide sequence and comprising, consisting essentially of and/or consisting of a nucleotide sequence of contiguous nucleotides identical or almost identical (e.g., 80%, 81%, 82%, 83%, 84%, 85%, 86%, 87%, 88%, 89%, 90%, 91%, 92%, 93%, 94%, 95%, 96%, 98%, 99% identical) to the reference nucleic acid or nucleotide sequence. Such a nucleic acid fragment according to the invention may be, where appropriate, included in a larger polynucleotide of which it is a constituent.

An “isolated” nucleic acid molecule or nucleotide sequence or nucleic acid construct or double stranded RNA molecule of the present invention is generally free of nucleotide sequences that flank the nucleic acid of interest in the genomic DNA of the organism from which the nucleic acid was derived (such as coding sequences present at the 5′ or 3′ ends). However, the nucleic acid molecule of this invention can include some additional bases or moieties that do not deleteriously affect the basic structural and/or functional characteristics of the nucleic acid. “Isolated” does not mean that the preparation is technically pure (homogeneous).

Thus, an “isolated nucleic acid” or “isolated nucleic acid molecule” is a nucleotide sequence (either DNA or RNA) that is present in a form or setting that is different from that in which it is found in nature and is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. Thus, a nucleic acid molecule found in nature that is removed from its native environment and transformed into a plant is still considered “isolated” even when incorporated into a genome of the resulting transgenic plant. It also includes a recombinant nucleic acid that is part of a hybrid nucleic acid encoding an additional polypeptide or peptide sequence.

The term “isolated” can further refer to a nucleic acid, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid, nucleotide sequence or polypeptide that is not naturally occurring as a fragment and would not be found as such in the natural state. “Isolated” does not mean that the preparation is technically pure (homogeneous), but it is sufficiently pure to provide the polypeptide or nucleic acid in a form in which it can be used for the intended purpose.

In representative embodiments of the invention, an “isolated” nucleic acid, nucleotide sequence, and/or polypeptide is at least about 5%, 10%, 15%, 20%, 25%, 30%, 40%, 50%, 60%, 70%, 75%, 80%, 85%, 90%, 95%, 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10.000-fold, 100.000-fold or more enrichment of the nucleic acid (w/w) is achieved as compared with the starting material.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

As used herein, the terms “substantially complementary” or “partially complementary mean that two nucleic acid sequences are complementary at least about 50%, 60%, 70%, 80% or 90% of their nucleotides. In some embodiments, the two nucleic acid sequences can be complementary at least at 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g. a different copy number, and/or under the control of different regulatory sequences than that found in nature.

As used herein, the terms “transformed” and “transgenic” refer to any plant, plant cell, callus, plant tissue, or plant part that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

The term “transgene” as used herein, refers to any nucleic acid sequence used in the transformation of a plant, animal, or other organism. Thus, a transgene can be a coding sequence, a non-coding sequence, a cDNA, a gene or fragment or portion thereof, a genomic sequence, a regulatory element and the like. A “transgenic” organism, such as a transgenic plant, transgenic microorganism, or transgenic animal, is an organism into which a transgene has been delivered or introduced and the transgene can be expressed in the transgenic organism to produce a product, the presence of which can impart an effect and/or a phenotype in the organism.

Different nucleic acids or polypeptides having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins.

As used herein, the terms “contacting,” “introducing” and “administering” are used interchangeably, and refer to a process by which dsRNA of the present invention or a nucleic acid molecule encoding a dsRNA of this invention is delivered to a cell (e.g., of a nematode), in order to inhibit or alter or modify expression of an essential target gene in the nematode. The dsRNA may be administered in a number of ways, including, but not limited to, direct introduction into a cell (i.e., intracellularly) and/or extracellular introduction into a cavity, interstitial space, or into the circulation of the nematode. Oral introduction can also be employed, wherein a dsRNA and/or a nucleic acid molecule encoding the dsRNA may be introduced by bathing the nematode in a solution containing the dsRNA and/or nucleic acid, or the dsRNA and/or nucleic acid may be present in food source. Methods for oral introduction include direct mixing of dsRNA and/or nucleic acid molecules with food of the nematode, as well as engineered approaches in which a species that is used as food is engineered to express a dsRNA, which is then fed to the organism to be affected. For example, the dsRNA may be applied to and/or sprayed onto a plant, and/or the dsRNA may be applied to soil in the vicinity of roots, taken up by the plant and/or the nematode, and/or a plant may be genetically engineered to express the dsRNA in an amount sufficient to kill some or all of the nematode to which the plant is exposed.

“Introducing” in the context of a plant cell or plant means presenting the nucleic acid molecule to the plant, plant part, and/or plant cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol. Thus, the term “transformation” as used herein refers to the introduction of a heterologous nucleic acid into a cell. Transformation of a cell may be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and plastid genome, and therefore includes integration of the nucleic acid into, for example, the chloroplast genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Embodiments of the invention are directed to expression cassettes designed to express the nucleic acids of the present invention. As used herein, “expression cassette” means a nucleic acid molecule having at least a control sequence operably linked to a nucleotide sequence of interest. In this manner, for example, plant promoters in operable interaction with the nucleotide sequences for the miRNAs of the invention are provided in expression cassettes for expression in a plant, plant part and/or plant cell.

As used herein, the term “promoter” refers to a region of a nucleotide sequence that incorporates the necessary signals for the efficient expression of a coding sequence. This may include sequences to which an RNA polymerase binds, but is not limited to such sequences and can include regions to which other regulatory proteins bind together with regions involved in the control of protein translation and can also include coding sequences.

Furthermore, a “promoter” of this invention is a promoter capable of initiating transcription in a cell of a plant. Such promoters include those that drive expression of a nucleotide sequence constitutively, those that drive expression when induced, and those that drive expression in a tissue- or developmentally-specific manner, as these various types of promoters are known in the art.

For purposes of the invention, the regulatory regions (i.e., promoters, transcriptional regulatory regions, and translational termination regions) can be native/analogous to the plant, plant part and/or plant cell and/or the regulatory regions can be native/analogous to the other regulatory regions. Alternatively, the regulatory regions may be heterologous to the plant (and/or plant part and/or plant cell) and/or to each other (i.e., the regulatory regions). Thus, for example, a promoter can be heterologous when it is operably linked to a polynucleotide from a species different from the species from which the polynucleotide was derived. Alternatively, a promoter can also be heterologous to a selected nucleotide sequence if the promoter is from the same/analogous species from which the polynucleotide is derived, but one or both (i.e., promoter and polynucleotide) are substantially modified from their original form and/or genomic locus, or the promoter is not the native promoter for the operably linked polynucleotide.

The choice of promoters to be used depends upon several factors, including, but not limited to, cell- or tissue-specific expression, desired expression level, efficiency, inducibility and selectability. For example, where expression in a specific tissue or organ is desired, a tissue-specific promoter can be used (e.g., a root specific promoter). In contrast, where expression in response to a stimulus is desired, an inducible promoter can be used. Where continuous expression is desired throughout the cells of a plant, a constitutive promoter can be used. It is a routine matter for one of skill in the art to modulate the expression of a nucleotide sequence by appropriately selecting and positioning promoters and other regulatory regions relative to that sequence.

Therefore, in some instances, constitutive promoters can be used. Examples of constitutive promoters include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert at al. (1987) Proc. Natl. Acad. Sci. USA 84:5745-5749), Adh promoter (Walker at al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter.

Moreover, tissue-specific regulated nucleic acids and/or promoters have been reported in plants. Thus, in some embodiments, tissue specific promoters can be used. Some reported tissue-specific nucleic acids include those encoding the seed storage proteins (such as (3-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Thus, the promoters associated with these tissue-specific nucleic acids can be used in the present invention. Additional examples of tissue-specific promoters include, but are not limited to, the root-specific promoters RCc3 (Jeong et al. Plant Physiol. 153:185-197 (2010)) and RB7 (U.S. Pat. No. 5,459,252), the lectin promoter (Lindstrom at al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal at al. (1992) Proc. Natl. Acad. Sci. USA 89:3654-3658), corn heat shock protein promoter (O'Dell at al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” 29-39 In: Genetic Engineering of Plants (Hollaender ed., Plenum Press 1983; and Poulsen at al. (1986) Mol. Gen. Genet. 205:193-200), Ti plasmid mannopine synthase promoter (Langridge et al, (1989) Proc. Natl. Acad. Sci. USA 86:3219-3223), Ti plasmid nopaline synthase promoter (Langridge at al. (1989), supra), petunia chalcone isomerase promoter (van Tunen at al. (1988) EMBO J. 7:1257-1263), bean glycine rich protein 1 promoter (Keller at al. (1989) Genes Dev. 3:1639-1646), truncated CaMV 35S promoter (O'Dell et al. (1985) Nature 313:810-812), potato patatin promoter (Wenzler at al. (1989) Plant Mol. Biol. 13:347-354), root cell promoter (Yamamoto at al. (1990) Nucleic Acids Res. 18:7449), maize zein promoter (Kriz at al. (1987) Mol. Gen. Genet. 207:90-98; Langridge et al. (1983) Cell 34:1015-1022; Reina at al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). Particularly useful for seed-specific expression is the pea vicilin promoter (Czako et al. (1992) Mol. Gen. Genet. 235:33-40; as well as U.S. Pat. No. 5,625,136). Other useful promoters for expression in mature leaves are those that are switched on at the onset of senescence, such as the SAG promoter from Arabidopsis (Gan et al. (1995) Science 270:1986-1988). In addition, promoters functional in plastids can be used. Non-limiting examples of such promoters include the bacteriophage T3 gene 9 5′ UTR and other promoters disclosed in U.S. Pat. No. 7,579,516. Other promoters useful with the present invention, include but are not limited to the S-E9 small subunit RuBP carboxylase promoter and the Kunitz trypsin inhibitor gene promoter (Kti3).

In some instances, inducible promoters can be used. Examples of inducible promoters include, but are not limited to, tetracycline repressor system promoters, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters. Other inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston at al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez at al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108.

In addition to the promoters described above, the expression cassette also can include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences and polyadenylation signal sequences.

A number of non-translated leader sequences derived from viruses also are known to enhance gene expression. Specifically, leader sequences from Tobacco Mosaic Virus (TMV, the “ω-sequence”), Maize Chlorotic Mottle Virus (MCMV) and Alfalfa Mosaic Virus (AMV) have been shown to be effective in enhancing expression (Gallie et al. (1987) Nucleic Acids Res. 15:8693-8711; and Skuzeski et al. (1990) Plant Mol. Biol. 15:65-79). Other leader sequences known in the art include, but are not limited to, picornavirus leaders such as an encephalomyocarditis (EMCV) 5′ noncoding region leader (Elroy-Stein et al. (1989) Proc. Natl. Acad. Sci. USA 86:6126-6130); potyvirus leaders such as a Tobacco Etch Virus (TEV) leader (Allison et al. (1986) Virology 154:9-20); Maize Dwarf Mosaic Virus (MDMV) leader (Allison et al. (1986), supra); human immunoglobulin heavy-chain binding protein (BiP) leader (Macejak & Samow (1991) Nature 353:90-94); untranslated leader from the coat protein mRNA of AMV (AMV RNA 4; Jobling & Gehrke (1987) Nature 325:622-625); tobacco mosaic TMV leader (Gallie et al. (1989) Molecular Biology of RNA 237-256); and MCMV leader (Lommel et al, (1991) Virology 81:382-385). See also, Della-Cioppa et al. (1987) Plant Physiol. 84:965-968.

The expression cassette also can optionally include a transcriptional and/or translational termination region (i.e., termination region) that is functional in plants. A variety of transcriptional terminators are available for use in expression cassettes and are responsible for the termination of transcription beyond the transgene and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 355 terminator, the tml terminator, the nopaline synthase terminator and the pea rbcs E9 terminator. These can be used in both monocotyledons and dicotyledons. In addition, a coding sequence's native transcription terminator can be used.

A signal sequence can be operably linked to nucleic acids of the present invention to direct the nucleotide sequence into a cellular compartment. In this manner, the expression cassette will comprise a nucleotide sequence encoding the miRNA operably linked to a nucleic acid sequence for the signal sequence. The signal sequence may be operably linked at the N- or C-terminus of the miRNA.

Regardless of the type of regulatory sequence(s) used, they can be operably linked to the nucleotide sequence of the miRNA. As used herein, “operably linked” means that elements of a nucleic acid construct such as an expression cassette are configured so as to perform their usual function. Thus, regulatory or control sequences (e.g., promoters) operably linked to a nucleotide sequence of interest are capable of effecting expression of the nucleotide sequence of interest. The control sequences need not be contiguous with the nucleotide sequence of interest, so long as they function to direct the expression thereof. Thus, for example, intervening untranslated, yet transcribed, sequences can be present between a promoter and a coding sequence, and the promoter sequence can still be considered “operably linked” to the coding sequence. A nucleotide sequence of the present invention (i.e., a miRNA) can be operably linked to a regulatory sequence, thereby allowing its expression in a cell and/or subject.

The expression cassette also can include a nucleotide sequence for a selectable marker, which can be used to select a transformed plant, plant part or plant cell. As used herein, “selectable marker” means a nucleic acid that when expressed imparts a distinct phenotype to the plant, plant part or plant cell expressing the marker and thus allows such transformed plants, plant parts or plant cells to be distinguished from those that do not have the marker. Such a nucleic acid may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleic acid encoding neo or nptII, which confers resistance to kanamycin, G418, and the like (Potrykus et al. (1985) Mol. Gen. Genet. 199:183-188); a nucleic acid encoding bar, which confers resistance to phosphinothricin; a nucleic acid encoding an altered 5-enolpyruvylshikimate-3-phosphate (EPSP) synthase, which confers resistance to glyphosate (Hinchee et al (1988) Biotech. 6:915-922); a nucleic acid encoding a nitrilase such as bxn from Klebsiella ozaenae that confers resistance to bromoxynil (Stalker et al (1988) Science 242:419-423); a nucleic acid encoding an altered acetolactate synthase (ALS) that confers resistance to imidazolinone, sulfonylurea or other ALS-inhibiting chemicals (EP Patent Application No, 154204); a nucleic acid encoding a methotrexate-resistant dihydrofolate reductase (DHFR) (Thillet et al. (1988) J. Biol. Chem. 263:12500-12508); a nucleic acid encoding a dalapon dehalogenase that confers resistance to dalapon; a nucleic acid encoding a mannose-6-phosphate isomerase (also referred to as phosphomannose isomerase (PMI)) that confers an ability to metabolize mannose (U.S. Pat. Nos. 5,767,378 and 5,994,629); a nucleic acid encoding an altered anthranilate synthase that confers resistance to 5-methyl tryptophan; and/or a nucleic acid encoding hph that confers resistance to hygromycin. One of skill in the art is capable of choosing a suitable selectable marker for use in an expression cassette.

Additional selectable markers include, but are not limited to, a nucleic acid encoding β-glucuronidase or uidA (GUS) that encodes an enzyme for which various chromogenic substrates are known; an R-locus nucleic acid that encodes a product that regulates the production of anthocyanin pigments (red color) in plant tissues (Dellaporta et al., “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleic acid encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleic acid encoding xylE that encodes a catechol dioxygenase (Zukowsky et al, (1983) Proc. Natl. Acad. Sci. USA 80:1101-1105); a nucleic acid encoding tyrosinase, an enzyme capable of oxidizing tyrosine to DOPA and dopaquinone, which in turn condenses to form melanin (Katz et al. (1983) J. Gen. Microbiol. 129:2703-2714); a nucleic acid encoding β-galactosidase, an enzyme for which there are chromogenic substrates; a nucleic acid encoding luciferase (lux) that allows for bioluminescence detection (Ow et al. (1986) Science 234:856-859); a nucleic acid encoding aequorin which may be employed in calcium-sensitive bioluminescence detection (Prasher et al, (1985) Biochem. Biophys. Res. Comm. 126:1259-1268); or a nucleic acid encoding green fluorescent protein (Niedz et al. (1995) Plant Cell Reports 14:403-406). One of skill in the art is capable of choosing a suitable selectable marker for use in an expression cassette.

An expression cassette of the present invention also can include nucleotide sequences for coding for other desired traits. Such sequences can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, the nucleotide sequences of interest can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleic acid molecule, nucleic acid construct, chimeric nucleic acid molecule, artificial microRNA precursor molecule and/or composition of this invention, provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853.

The expression cassette also can include a coding sequence for one or more polypeptides for agronomic traits that primarily are of benefit to a seed company, grower or grain processor, for example, bacterial pathogen resistance, fungal resistance, herbicide resistance, insect resistance, nematode resistance and virus resistance. See, e.g., U.S. Pat. Nos. 5,304,730; 5,495,071; 5,569,823; 6,329,504 and 6,337,431. The trait also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various traits of interest, as well as methods for introducing these traits into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Application Publication No. 2001/0016956. See also, on the World Wide Web at lifesci.sussex.ac.uk/home/Neil_Crickmore/Bt/.

Numerous nucleotide sequences are known to enhance expression from within a transcriptional unit, and these sequences can be used in conjunction with the nucleotide sequences of this invention to increase or enhance expression in transgenic plants. For example, introns of the maize Adhl gene and Intron 1 have been shown to enhance gene expression. See, e.g., Callis et al, (1987) Genes Develop. 1:1183-1200.

In some embodiments of the present invention, the expression cassette can comprise an expression control sequence operatively linked to a nucleotide sequence that is a template for one or both strands of the dsRNA. The dsRNA template comprises (a) a first (antisense) stand having a sequence complementary to from about 18 to about 25 consecutive nucleotides of the nucleotide sequence of SEQ ID NO:931; and (b) a second (sense) strand having a nucleotide sequence fully complementary or substantially complementary to the first strand. In further embodiments, a promoter can flank either end of the template nucleotide sequence, wherein the promoters drive expression of each individual DNA strand, thereby generating two complementary (or substantially complementary) RNAs that hybridize and form the dsRNA. In alternative, embodiments, the nucleotide sequence is transcribed into both strands of the dsRNA on one transcription unit, wherein the sense strand is transcribed from the 5′ end of the transcription unit and the antisense strand is transcribed from the 3′ end, wherein the two strands are separated by about 3 to about 500 basepairs, and wherein after transcription, the RNA transcript folds on itself to form a short hairpin RNA (shRNA) molecule.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleic acid sequences have at least 60%, 70%, 80% or 90% sequence identity. In some embodiments, the two nucleic acid sequences can have at least 85%, 90%, 95%, 96%, 97%, 98%, 99% or 100% of sequence identity.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48:1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

Accordingly, the present invention further provides nucleotide sequences having significant sequence identity to the nucleotide sequences of the present invention. Significant sequence similarity or identity means at least 70%, 75%, 80%, 81%, 82%, 83%, 84%, 85%, 86%, 87%, 88%, 89%, 90%, 91%, 92%, 93%, 94%, 96%, 97%, 98%, 99% and/or 100% similarity or identity with another nucleotide sequence.

The following examples are not intended to limit the scope of the claims to the invention, but are rather intended to be exemplary of certain embodiments. Any variations in the exemplified methods that occur to the skilled artisan are intended to fall within the scope of the present invention. As will be understood by one skilled in the art, there are several embodiments and elements for each aspect of the claimed invention, and all combinations of different elements are hereby anticipated, so the specific combinations exemplified herein are not to be construed as limitations in the scope of the invention as claimed. If specific elements are removed or added to the group of elements available in a combination, then the group of elements is to be construed as having incorporated such a change.

### EXAMPLES

### Example 1

### siRNAs Targeting Various Regions of The Hg-Rps23 EST

Summary.

Four different small interfering RNA (siRNA) duplexes were designed to target various regions of the Hg-rps23 EST (GenBank® Database Accession Number BF014259; SEQ ID NO:931) of the soybean cyst nematode (SCN). The second stage juveniles (J2) of SCN were then soaked in these chemically synthesized siRNA duplexes, followed by subsequent nematode reproduction assay on host plants. Two of the siRNA duplexes were shown to immobilize the J2 and reduce the number of cysts formed on the host plant.

Experimental Approaches.

Four siRNA duplexes that target the Hg-rps23 EST of SCN were designed and chemically synthesized. The algorithm was based on the online tool at http://www/genelink.com. The sequences of the siRNA duplexes are: 1. si-rps23-1, sense strand: GAAGCGCAAUUUCCGAGAATT (SEQ ID NO:927 with 3′ TT included (Table 3)), antisense strand: UUCUCGGAAAUUGCGCUUCTT (SEQ ID NO:863 with 3′ TT included); 2. si-rps23-2, sense strand AUUGCAAAUUGUUUUGAAATT (SEQ ID NO:928 with 3′ TT included (Table 3)), antisense strand: UUUCAGAGCAAUUUGCAAUTT (SEQ ID NO:836 with 3′ TT included); 3. si-rps23-3, sense strand UUGCAUCCUUGGUGAUUAATT (SEQ ID NO:929 with 3′ TT included (Table 3)), antisense strand: UUGGUCGCCAAGGAUGCAATT (SEQ ID NO:740 with 3′ TT included); 4. si-rps23-4, sense strand ACCUGAAGAAGUUGAACAATT (SEQ ID NO:930 with 3′ TT included (Table 3)), antisense strand: UUGUUCAACUUCUUCAGGUTT (SEQ ID NO:669 with 3′ TT included).

One control was a negative siRNA duplex (si-control) from GeneLink (Catalog #27-6411-20), sense strand and antisense strand sequences unknown. Another control was H₂O.

Freshly hatched SCN J2s were soaked in the siRNA solutions in a 96-well plate under the following conditions: 250 J2/well with each well containing a different siRNA duplex; siRNA duplex concentration=0.5 μg/μl, octopamine concentration=50 μM, temperature=26° C.

After four days of soaking in darkness, the J2s were observed. The results were: H₂O control: most J2s were actively moving; si-control: most J2s were actively moving; si-rps23-1: most J2s were immobilized; si-rps23-2: most J2s were actively moving, some immobilized; si-rps23-3: some J2s were actively moving, some immobilized; and si-rps23-4: most J2s were immobilized

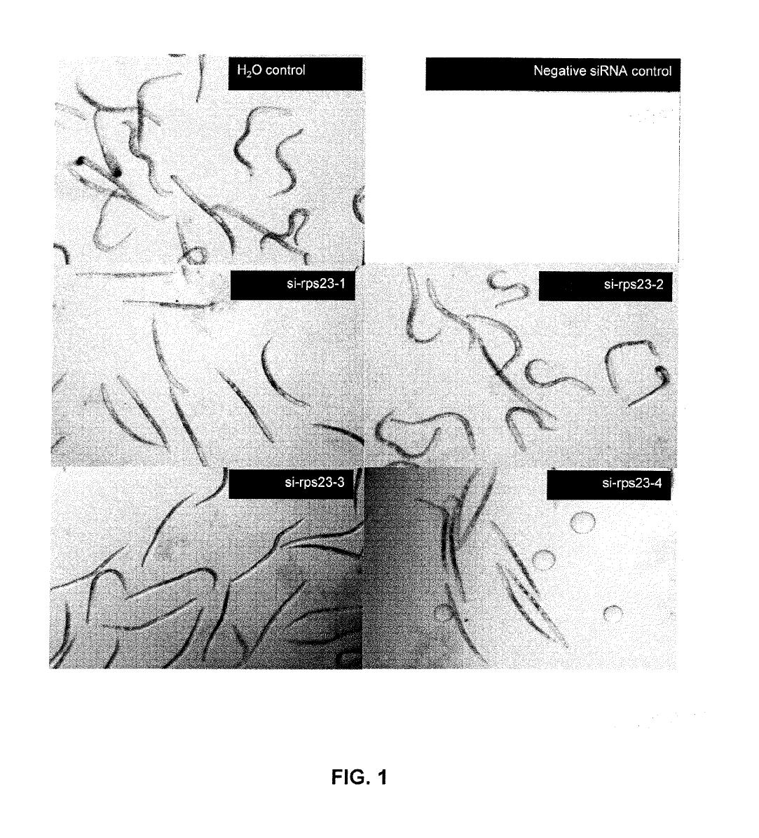

FIG. 1 shows photographs of the J2s in each treatment. Curly J2 indicates movement, and straight or “C” shaped J2 indicates inactivity. It is clear from the results that the si-rps23-1 and si-rps23-4 can immobilize the J2.

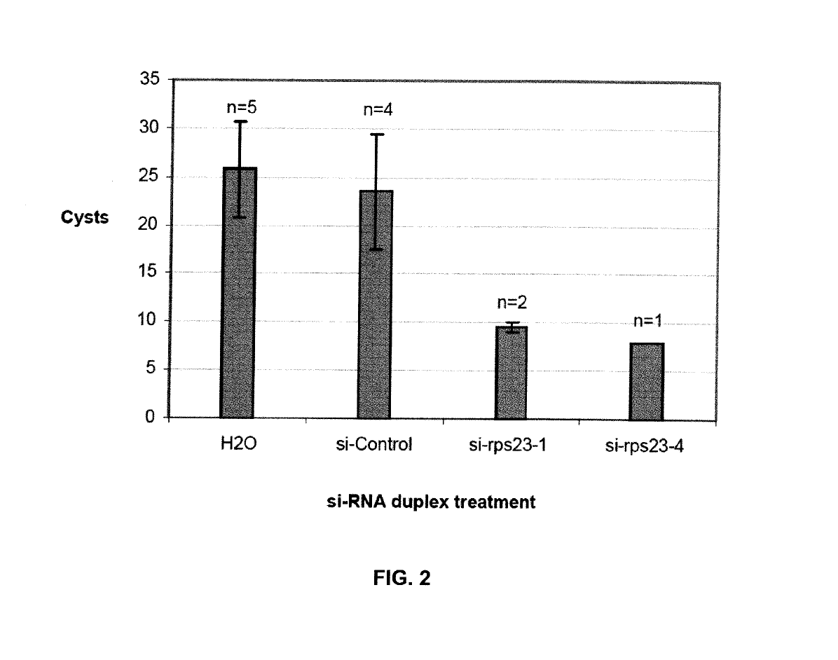

In another repeat experiment, the above controls and si-rps23-1 and si-rps23-4 were used to treat SCN J2s under the same conditions. Equal numbers of J2s were treated in each treatment, with similar results observed 4 days after treatment. The nematodes were then inoculated onto soybean seedlings growing in pouches and cultured at 26° C. with 16 hr/day lighting. Each pouch contains one soybean seedling and was inoculated with J2 from one treatment. One month later, the numbers of cysts on each pouch were counted. The cyst numbers were then plotted against the siRNA treatment and presented in FIG. 2 (n=# of replicates).

It was concluded from these experiments that the si-rps23-1 and the si-rps23-4 duplexes were able to immobilize the J2 of SCN and significantly reduced cyst formation on the host plant.

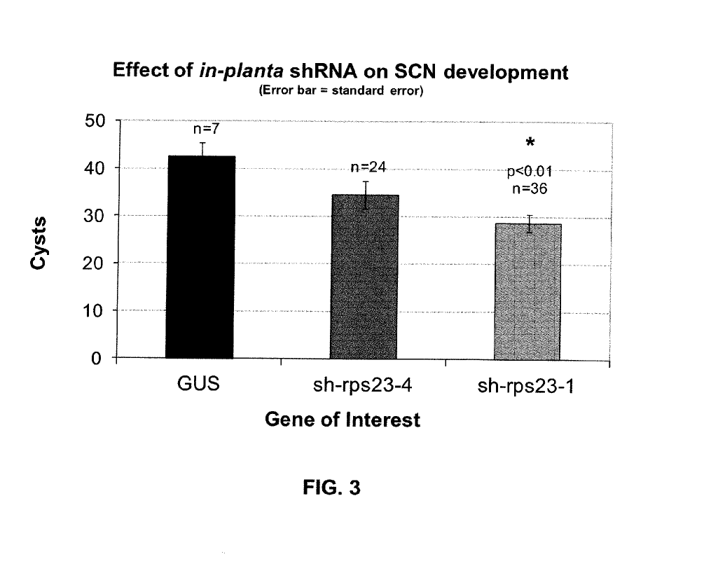

The si-rps23-1 and si-rps23-4 were expressed in the manner of short hairpin RNA (shRNA) in transgenic soybean hairy root. The shRNA sequence for sh-rps23-1 is gaagcgcaatttccgagaatatcaagagtattctcggaaattgcgcttctgtttttt (SEQ ID NO:932), while the shRNA sequence for sh-rps23-4 is acctgaagaagttgaacaatatcaagagtattgttcaacttcttcaggttgttttttt (SEQ ID NO:933). Soybean cyst nematode assays were conducted and the number of cysts on these transgenic roots was compared to the negative control. Results are illustrated in FIG. 3. The results indicated that the average number of cysts in the hairy roots over-expressing sh-rps23-1 are significantly lower than the control roots over-expressing the GUS gene.

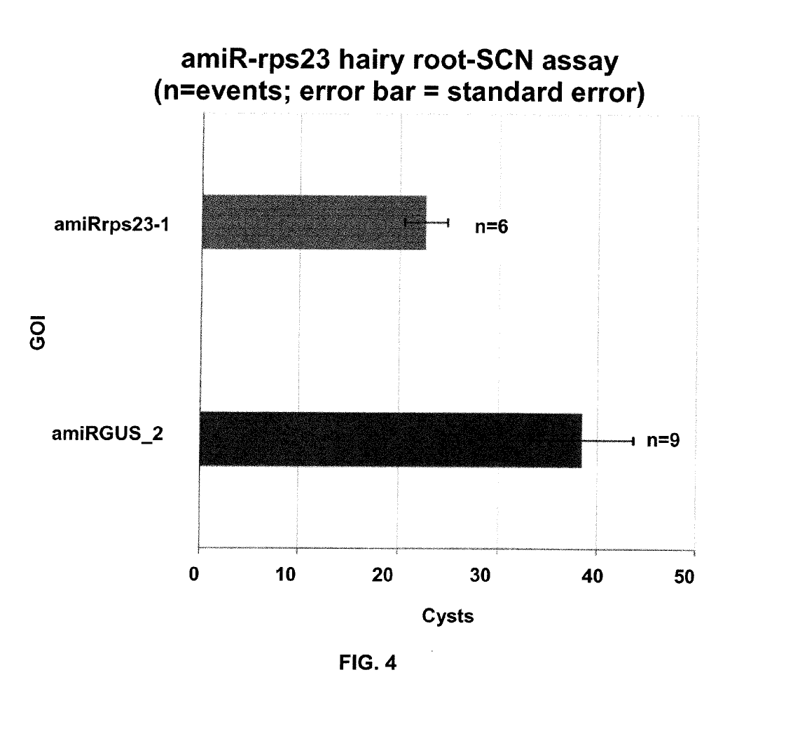

Another approach was taken to overexpress si-rps23-1 in the manner of artificial microRNA (amiRNA). Soybean microRNA precursor, gma-MIR164, was used as the backbone. The miR164/miR164* sequence on this precursor was replaced by si-rps23-1/si-rps23-1* sequence, while the mismatch positions on the miR164/miR164* duplex were maintained in the si-rps23-1/si-rps23-1* sequence. The artificial miRNA was named amiRrps23-1, and its sequence is ggatccagctccttgtttctcggaaattgcgcttcttagtctcttggatctcaaatgccactgaacccaagaagcgcaacctccgagaaca acacgggtttgagctc (SEQ ID NO:934). The amiRrps23-1 was transformed into soybean hairy roots, and multiple events were inoculated with the soybean cyst nematode J2s. The nematodes were allowed to develop into cysts on the root, and the average number of cysts on different events were compared to the control. These results are shown in FIG. 4. The results indicated that the average number of cysts in the hairy roots over-expressing amiR-rps23-1 are significantly lower than the control roots over-expressing amiR-GUS-2.

### Example 2

### Expression of Artificial microRNAs in Plant Hosts to Silence Target Genes in Pests/Pathogens

Designing the artificial microRNA. The design of the artificial microRNA (amiRNA) for expression of anti-pest small RNA in plant host cell is as described in Schwab et al. (“Highly specific gene silencing by artificial microRNAs in Arabidopsis” The Plant Cell 18:1121-1133 (2006), the entire contents of which are incorporated by reference herein for teachings of the use of artificial microRNAs), in which amiRNAs were designed to target individual genes or groups of endogenous genes in a plant cell.

For the studies of this invention, we chose the soybean miRNA precursor gma-MIR164 as the backbone of the amiRNA. The sequence of gma-MIR164 is as follows: agcuccuuguuggagaagcagggcacgugcaagucucuuggaucucaaaugccacugaacccuuugcacgugcuccccu ucuccaacacggguuu (SEQ ID NO:935). The folding structure of the transcript is as follows:

```
- u u ca --uc -u aucu
  agc cc uguuggagaag gggcacgugcaag uc ugg c
  uug gg acaaccucuuc cucgugcacguuu ag acc a
u - c cc ccca uc guaa
```

After processing by dicer, the miR164/miR164* duplex will be generated from the precursor, and further processing will generate the mature guiding strand miRNA164 and the passenger strand miR164*.

To design the amiRNA, the above miR164/miR164* strands are replaced with anti-SCN siRNA/siRNA* strands, while keeping the rest of the precursor.

As an example, miR164/miR164* strands were replaced with siRNA/siRNA* that targets the soybean cyst nematode (SCN) hg-rps23 gene. In in vitro soaking experiments, the siRNA duplex si-rps23-1/si-rps23-1* have been proven to immobilize the SCN J2s. The sequences of the si-rps23-1/si-rps23-1* duplex are:

(SEQ ID NO: 863)
si-rps23-1: uucucggaaauugcgcuucuu
(SEQ ID NO: 927; Table 3)
si-rps23-1*: gaagcgcaauuuccgagaa

In the miR164/miR164* duplex, there is a ca/cc mismatch between the two strands in the middle, which may be important for miRNA processing, therefore, the sequence of si-rps23*-1 was also mutated to generate a mismatch in the same position. The mutated si-rps23-1* sequence is: gaagcgcaaccuccgagaa (SEQ ID NO:936).

After replacing the miR164/miR164* in the gma-MIR164 precursor with the sequence of si-rps23-1/si-rps23-1*, the sequence of the amiRNA (aMIR164-rps23-1) is: agcuccuuguuucucggaaauugcgcuucuuagucucuuggaucucaaaugccacugaacccaagaagcgcaaccuccga gaacaacacggguuu (SEQ ID NO:937) and the folding structure of the amiRNA precursor transcript is as follows:

```
- u u -- aa a-|c u- aucu
  agc cc ugu uucucgga uugcgcuucuu gu uc ugg c
  uug gg aca aagagccu aacgcgaagaa ca ag acc a
u - c ac cc cc\hat{ }- uc guaa
```

### Transgenic Root Generation.

The purpose of this step is to generate transgenic soybean roots to overexpress the si-rps23-1 small RNA.
1. The above amiRNA (aMIR164-rps23-1) was cloned behind the CMP promoter into a binary vector.
2. The binary vector was then transformed into Agrobacterium rhizogenes strain K599.
3. The A. rhizogenes K599 strain carrying the binary vector was inoculated onto soybean cotyledons and transgenic hairy roots were induced a few weeks later.

### Detection of Si-Rps23-1 in Transgenic Roots.

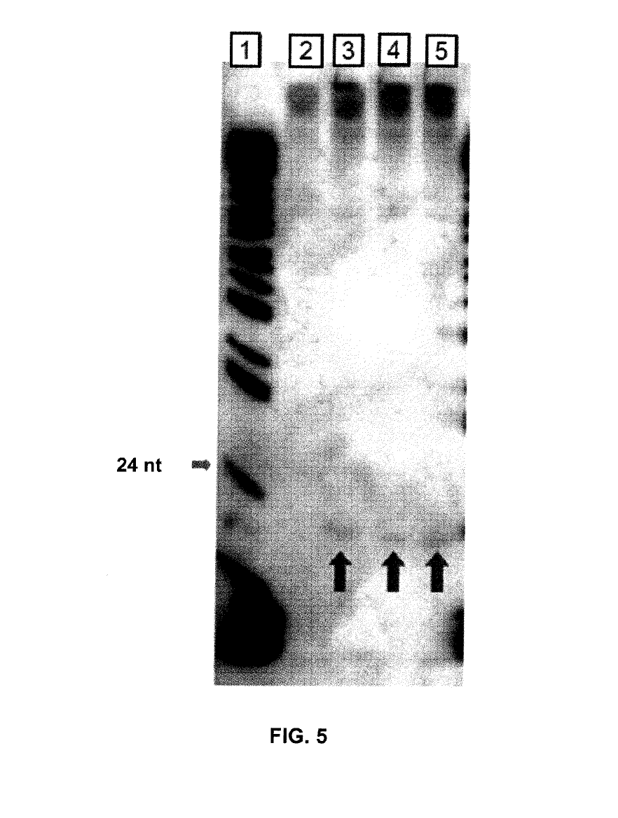

The purpose of this step is to detect the expression of si-rps23-1 in transgenic soybean roots.
1. RNA was extracted from transgenic soybean roots expressing the above amiRNA precursor.
2. Northern blot analysis was conducted to detect the si-rps23-1 small RNA, using a probe that specifically binds to it. The results in FIG. 5 indicate that the si-rps23-1 (arrows) was generated in hairy root samples (lane 3, 4, 5). Lane 2=negative control roots, Lane 1=molecular marker.

### Nematode Bioassay on Transgenic Roots.

The purpose of this step is to check the effect of si-rps23-1 on the reproduction of SCN on transgenic roots.
1. Transgenic roots overexpressing the si-rps23-1 were infected with the second stage juveniles (J2s) of SCN. As control, transgenic roots overexpressing an amiRNA targeting the GUS gene were also infected with J2s of SCN.
2. The roots and nematodes were cultured for a month, and the numbers of cysts formed on the roots were compared between the two constructs. Table 4 shows the summary of the comparison of mean cysts. Anova test indicates that the average cysts form on the transgenic roots overexpressing the amiR164-rps23-1 is significantly lower than that on the transgenic roots overexpressing the amiR164-GUS (p<0.05).

### Summary.

An anti-pest small RNA was designed and overexpressed in the form of artificial microRNA, using the context of plant miRNA. Northern blot indicated that the small RNA was generated in the plant cell, and bioassay indicated that the small RNA was able to reduce pest reproduction.

### Example 3

### Nematode Assay on Transgenic Plants Over-Expressing Si-Rps23-1

The sh-rps23-1 described in Example 1 was transformed into soybean cultivar Williams 82 to produce transgenic soybean plants. This was accomplished by using immature seed targets of variety Williams 82 via Agrobacterium tumefaciens-mediated transformation using explant materials and media recipes as described in Hwang et al 2008 (PCT Publication No. WO/08112044) and Que et al (PCT Publication No. WO/08112267) except where noted below. Using this method, genetic elements within the left and right border regions of the transformation plasmid are efficiently transferred and integrated into the genome of the plant cell, while genetic elements outside these border regions are generally not transferred. Maturing soybean pods were harvested from greenhouse grown plants, sterilized with diluted bleach solution and rinsed with sterile water. Immature seeds were then excised from seed pods and rinsed with sterile water briefly. Explants were prepared from sterilized immature seeds as described in Hwang et al 2008 (PCT Publication No. WO/08112044) and infected with A. tumefaciens strain EHA101 harboring the transformation binary vector and allowed to incubate for an additional 30 to 240 minutes. Excess A. tumefaciens suspension was then removed by aspiration and explants were moved to plates containing a non-selective co-culture medium. Explants were co-cultured with the remaining A. tumefaciens at 23° C. for 4 days in the dark. Explants were then transferred to recovery and regeneration medium supplemented with an antibiotic mixture consisting of ticarcillin (75 mg/L), cefotaxime (75 mg/L) and vancomycin (75 mg/l) and incubated in the dark for seven days. Explants were then transferred to regeneration medium containing hygromycin B (3 to 6 mg/L) and a mixture of antibiotics consisting of ticarcillin (75 mg/L), cefotaxime (75 mg/L) and vancomycin (75 mg/l) to inhibit and kill A. tumefaciens. Shoot elongation was carried out in elongation media containing the selection agent. Regenerated plantlets were transplanted to soil as described (PCT Publication No. WO/08112267) and tested for the presence of both the selection marker and the CMP promoter sequences by TaqMan PCR analysis (Ingham et al., 2001). This screen allows for the selection of transgenic events that carry the T-DNA and are free of vector backbone DNA. Plants positive for the selection gene and CMP sequences and negative for the spec gene were transferred to the greenhouse.

When the roots are about 2-3 inches, plants are then transplanted into 1 gallon pots using Fafard #3 soil and ⅛ cup (30 grams) of incorporated Osmocote Plus 15-9-12. They are watered in thoroughly and placed in the cubicle under florescent lighting set to a 16-hour day. The temperature settings are 85° F.—day and 70° F.—night. They are watered once daily. After secondary Taqman® sampling has been done, the plants are then placed on automatic drip and watered twice daily. The lighting is a combination of Metal Halide and Sodium Vapor fixtures with 400 and 1000 watt bulbs. These are scheduled for a 10-hour day. Temperatures are set at 79° F.—day, 70° F.—night. Humidity is ambient. The plants are maintained in this fashion until pods reach maturity. The pods are then harvested, placed in a paper bag, air-dried 2 days, and then machine dried at 80° F. for 2 more days. The pods are shelled and the T1 seeds are harvested and stored at 4° C. and 20% humidity until future assays.

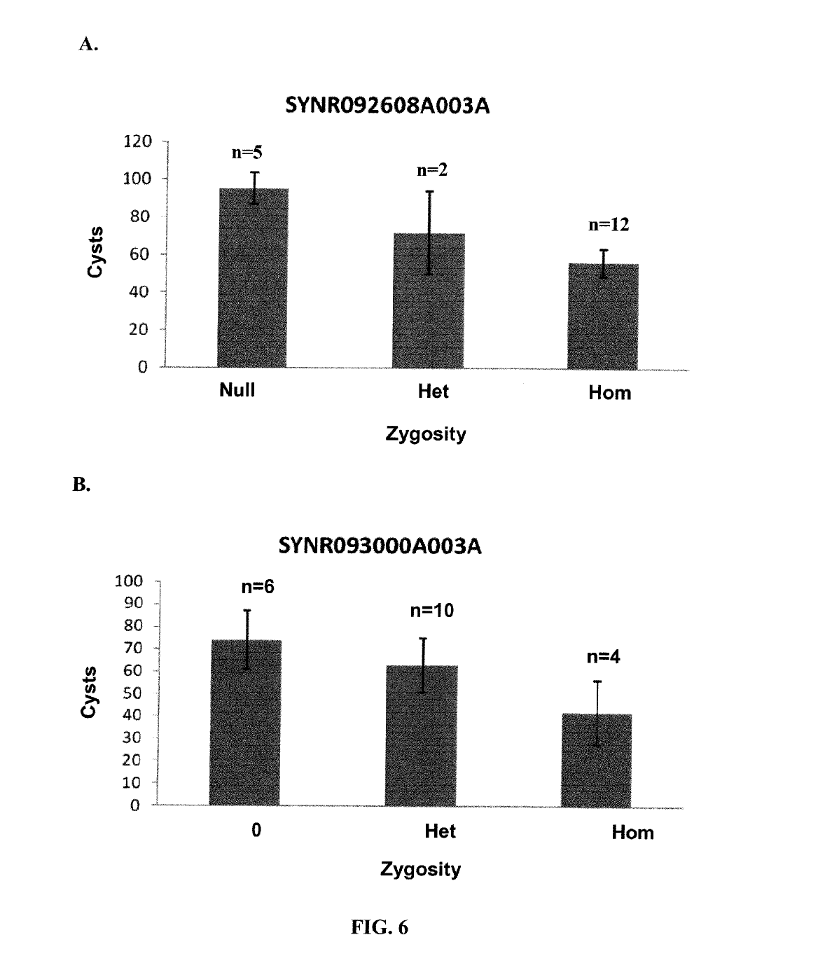

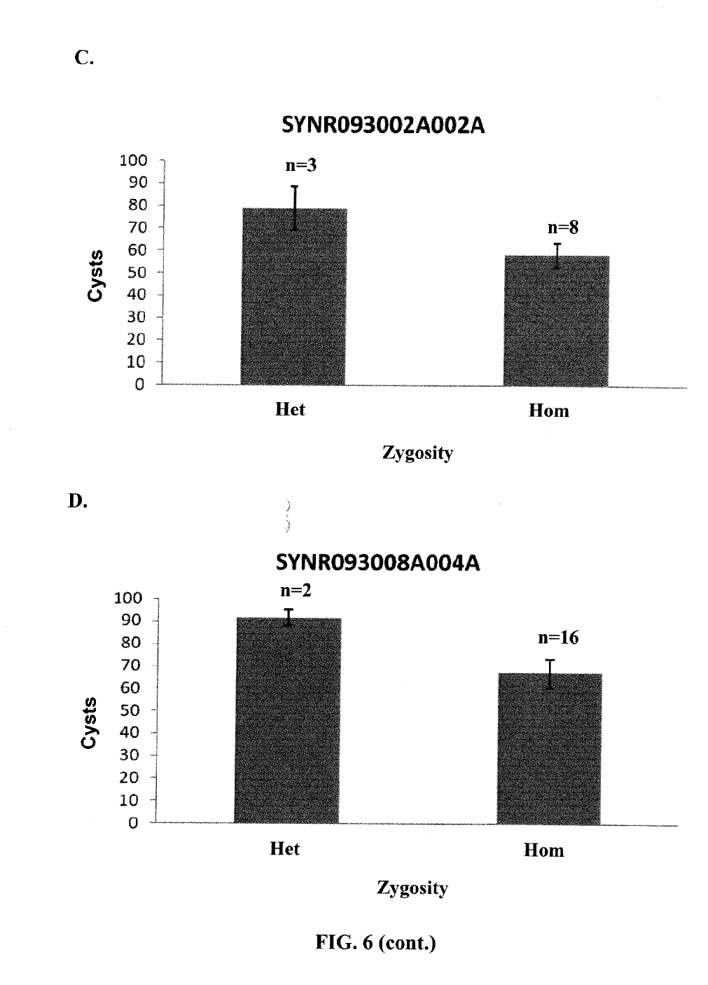

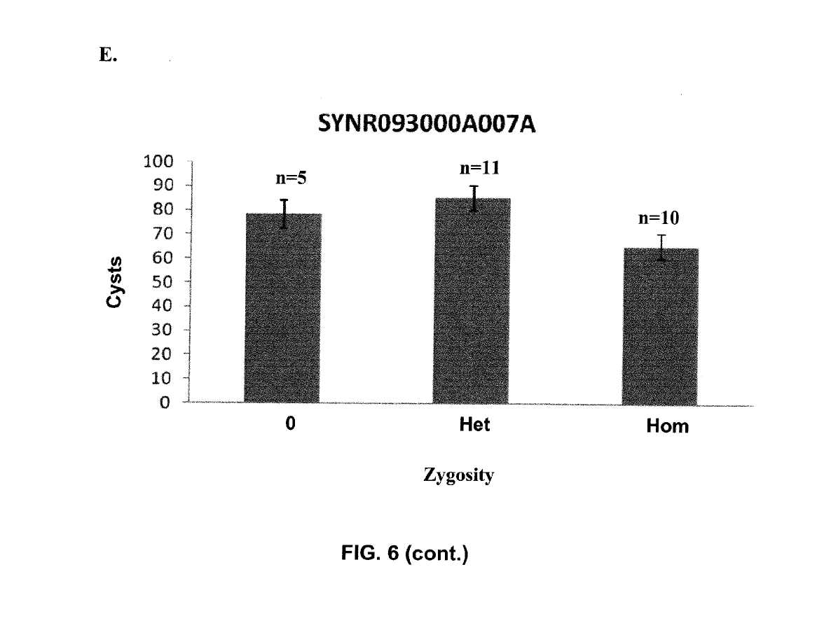

Forty T1 seeds from each of 15 T0 events were germinated in wet paper towel at 24° C. for 5 days. The germinated seedlings with 1.5 inches or longer root were transplanted into wet germination pouches with one seedling per pouch, and cultured at 24° C. for 24 hours. Each seedling was then inoculated with 1 ml of water containing 500 J2 of SCN. The seedlings were then cultured at 24° C. with 16 hours/day of lighting for 35 days, during which seedlings with fungal contamination were discarded. At 21 days after SCN inoculation, the leaves of each seedling were sampled by Taqman® assay of the zygosity of the prAR6 promoter. Since the prAR6 is immediately upstream of the sh-rps23-1 gene on the T-DNA, its copy number likely represents that of the sh-rps23-1 gene. Based on the copy number of the transgene, the zygosity of the T1 is determined as: Null (0 copy); Heterozygous (1 copy); Homozygous (2 or more copies). At 35 days after SCN inoculation, the number of cysts on each seedling was counted. The average numbers of cysts of the null, heterozygous, and homozygous plants of the same T0 event were compared. As shown in FIG. 6, the average number of cysts of homozygous plants of the same events is reduced compared to either the null or heterozygous plants.

All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

TABLE 1
siRNA target sequences of hg-rps-23
(SEQ ID NO: 1) caaaatcacacgtgaccag
(SEQ ID NO: 2) aaaatcacacgtgaccagc
(SEQ ID NO: 3) aaatcacacgtgaccagct
(SEQ ID NO: 4) aatcacacgtgaccagctg
(SEQ ID NO: 5) atcacacgtgaccagctga
(SEQ ID NO: 6) tcacacgtgaccagctgaa
(SEQ ID NO: 7) cacacgtgaccagctgaac
(SEQ ID NO: 8) acacgtgaccagctgaacg
(SEQ ID NO: 9) cacgtgaccagctgaacga
(SEQ ID NO: 10) acgtgaccagctgaacgag
(SEQ ID NO: 11) cgtgaccagctgaacgaga
(SEQ ID NO: 12) gtgaccagctgaacgagag
(SEQ ID NO: 13) tgaccagctgaacgagagt
(SEQ ID NO: 14) gaccagctgaacgagagtg
(SEQ ID NO: 15) accagctgaacgagagtgt
(SEQ ID NO: 16) ccagctgaacgagagtgtg
(SEQ ID NO: 17) cagctgaacgagagtgtgg
(SEQ ID NO: 18) agctgaacgagagtgtggc
(SEQ ID NO: 19) gctgaacgagagtgtggct
(SEQ ID NO: 20) ctgaacgagagtgtggctg
(SEQ ID NO: 21) tgaacgagagtgtggctga
(SEQ ID NO: 22) gaacgagagtgtggctgaa
(SEQ ID NO: 23) aacgagagtgtggctgaaa
(SEQ ID NO: 24) acgagagtgtggctgaaat
(SEQ ID NO: 25) cgagagtgtggctgaaatc
(SEQ ID NO: 26) gagagtgtggctgaaatct
(SEQ ID NO: 27) agagtgtggctgaaatctt
(SEQ ID NO: 28) gagtgtggctgaaatcttg
(SEQ ID NO: 29) agtgtggctgaaatcttga
(SEQ ID NO: 30) gtgtggctgaaatcttgaa
(SEQ ID NO: 31) tgtggctgaaatcttgaaa
(SEQ ID NO: 32) gtggctgaaatcttgaaac
(SEQ ID NO: 33) tggctgaaatcttgaaaca
(SEQ ID NO: 34) ggctgaaatcttgaaacaa
(SEQ ID NO: 35) gctgaaatcttgaaacaat
(SEQ ID NO: 36) ctgaaatcttgaaacaatc
(SEQ ID NO: 37) tgaaatcttgaaacaatcc
(SEQ ID NO: 38) gaaatcttgaaacaatccc
(SEQ ID NO: 39) aaatcttgaaacaatccca
(SEQ ID NO: 40) aatcttgaaacaatcccaa
(SEQ ID NO: 41) atcttgaaacaatcccaag
(SEQ ID NO: 42) tcttgaaacaatcccaaga
(SEQ ID NO: 43) cttgaaacaatcccaagag
(SEQ ID NO: 44) ttgaaacaatcccaagaga
(SEQ ID NO: 45) tgaaacaatcccaagagaa
(SEQ ID NO: 46) gaaacaatcccaagagaag
(SEQ ID NO: 47) aaacaatcccaagagaaga
(SEQ ID NO: 48) aacaatcccaagagaagaa
(SEQ ID NO: 49) acaatcccaagagaagaag
(SEQ ID NO: 50) caatcccaagagaagaagc
(SEQ ID NO: 51) aatcccaagagaagaagcg
(SEQ ID NO: 52) atcccaagagaagaagcgc
(SEQ ID NO: 53) tcccaagagaagaagcgca
(SEQ ID NO: 54) cccaagagaagaagcgcaa
(SEQ ID NO: 55) ccaagagaagaagcgcaat
(SEQ ID NO: 56) caagagaagaagcgcaatt
(SEQ ID NO: 57) aagagaagaagcgcaattt
(SEQ ID NO: 58) agagaagaagcgcaatttc
(SEQ ID NO: 59) gagaagaagcgcaatttcc
(SEQ ID NO: 60) agaagaagcgcaatttccg
(SEQ ID NO: 61) gaagaagcgcaatttccga
(SEQ ID NO: 62) aagaagcgcaatttccgag
(SEQ ID NO: 63) agaagcgcaatttccgaga
(SEQ ID NO: 64) gaagcgcaatttccgagaa
(SEQ ID NO: 65) aagcgcaatttccgagaaa
(SEQ ID NO: 66) agcgcaatttccgagaaac
(SEQ ID NO: 67) gcgcaatttccgagaaacg
(SEQ ID NO: 68) cgcaatttccgagaaacga
(SEQ ID NO: 69) gcaatttccgagaaacgat
(SEQ ID NO: 70) caatttccgagaaacgatt
(SEQ ID NO: 71) aatttccgagaaacgattg
(SEQ ID NO: 72) atttccgagaaacgattga
(SEQ ID NO: 73) tttccgagaaacgattgaa
(SEQ ID NO: 74) ttccgagaaacgattgaat
(SEQ ID NO: 75) tccgagaaacgattgaatt
(SEQ ID NO: 76) ccgagaaacgattgaattg
(SEQ ID NO: 77) cgagaaacgattgaattgc
(SEQ ID NO: 78) gagaaacgattgaattgca
(SEQ ID NO: 79) agaaacgattgaattgcaa
(SEQ ID NO: 80) gaaacgattgaattgcaaa
(SEQ ID NO: 81) aaacgattgaattgcaaat
(SEQ ID NO: 82) aacgattgaattgcaaatt
(SEQ ID NO: 83) acgattgaattgcaaattg
(SEQ ID NO: 84) cgattgaattgcaaattgc
(SEQ ID NO: 85) gattgaattgcaaattgct
(SEQ ID NO: 86) attgaattgcaaattgctc
(SEQ ID NO: 87) ttgaattgcaaattgctct
(SEQ ID NO: 88) tgaattgcaaattgctctg
(SEQ ID NO: 89) gaattgcaaattgctctga
(SEQ ID NO: 90) aattgcaaattgctctgaa
(SEQ ID NO: 91) attgcaaattgctctgaaa
(SEQ ID NO: 92) ttgcaaattgctctgaaaa
(SEQ ID NO: 93) tgcaaattgctctgaaaaa
(SEQ ID NO: 94) gcaaattgctctgaaaaac
(SEQ ID NO: 95) caaattgctctgaaaaact
(SEQ ID NO: 96) aaattgctctgaaaaacta
(SEQ ID NO: 97) aattgctctgaaaaactac
(SEQ ID NO: 98) attgctctgaaaaactacg
(SEQ ID NO: 99) ttgctctgaaaaactacga
(SEQ ID NO: 100) tgctctgaaaaactacgac
(SEQ ID NO: 101) gctctgaaaaactacgacc
(SEQ ID NO: 102) ctctgaaaaactacgaccc
(SEQ ID NO: 103) tctgaaaaactacgaccca
(SEQ ID NO: 104) ctgaaaaactacgacccac
(SEQ ID NO: 105) tgaaaaactacgacccaca
(SEQ ID NO: 106) gaaaaactacgacccacag
(SEQ ID NO: 107) aaaaactacgacccacaga
(SEQ ID NO: 108) aaaactacgacccacagaa
(SEQ ID NO: 109) aaactacgacccacagaag
(SEQ ID NO: 110) aactacgacccacagaagg
(SEQ ID NO: 111) actacgacccacagaagga
(SEQ ID NO: 112) ctacgacccacagaaggac
(SEQ ID NO: 113) tacgacccacagaaggaca
(SEQ ID NO: 114) acgacccacagaaggacaa
(SEQ ID NO: 115) cgacccacagaaggacaag
(SEQ ID NO: 116) gacccacagaaggacaagc
(SEQ ID NO: 117) acccacagaaggacaagcg
(SEQ ID NO: 118) cccacagaaggacaagcgt
(SEQ ID NO: 119) ccacagaaggacaagcgtt
(SEQ ID NO: 120) cacagaaggacaagcgttt
(SEQ ID NO: 121) acagaaggacaagcgtttc
(SEQ ID NO: 122) cagaaggacaagcgtttca
(SEQ ID NO: 123) agaaggacaagcgtttcag
(SEQ ID NO: 124) gaaggacaagcgtttcagt
(SEQ ID NO: 125) aaggacaagcgtttcagtg
(SEQ ID NO: 126) aggacaagcgtttcagtgg
(SEQ ID NO: 127) ggacaagcgtttcagtgga
(SEQ ID NO: 128) gacaagcgtttcagtggaa
(SEQ ID NO: 129) acaagcgtttcagtggaac
(SEQ ID NO: 130) caagcgtttcagtggaact
(SEQ ID NO: 131) aagcgtttcagtggaactg
(SEQ ID NO: 132) agcgtttcagtggaactgt
(SEQ ID NO: 133) gcgtttcagtggaactgtt
(SEQ ID NO: 134) cgtttcagtggaactgtta
(SEQ ID NO: 135) gtttcagtggaactgttag
(SEQ ID NO: 136) tttcagtggaactgttaga
(SEQ ID NO: 137) ttcagtggaactgttagac
(SEQ ID NO: 138) tcagtggaactgttagact
(SEQ ID NO: 139) cagtggaactgttagactg
(SEQ ID NO: 140) agtggaactgttagactga
(SEQ ID NO: 141) gtggaactgttagactgaa
(SEQ ID NO: 142) tggaactgttagactgaag
(SEQ ID NO: 143) ggaactgttagactgaagc
(SEQ ID NO: 144) gaactgttagactgaagca
(SEQ ID NO: 145) aactgttagactgaagcac
(SEQ ID NO: 146) actgttagactgaagcaca
(SEQ ID NO: 147) ctgttagactgaagcacat
(SEQ ID NO: 148) tgttagactgaagcacatc
(SEQ ID NO: 149) gttagactgaagcacatcc
(SEQ ID NO: 150) ttagactgaagcacatccc
(SEQ ID NO: 151) tagactgaagcacatccct
(SEQ ID NO: 152) agactgaagcacatccctc
(SEQ ID NO: 153) gactgaagcacatccctcg
(SEQ ID NO: 154) actgaagcacatccctcgt
(SEQ ID NO: 155) ctgaagcacatccctcgtc
(SEQ ID NO: 156) tgaagcacatccctcgtcc
(SEQ ID NO: 157) gaagcacatccctcgtccg
(SEQ ID NO: 158) aagcacatccctcgtccga
(SEQ ID NO: 159) agcacatccctcgtccgaa
(SEQ ID NO: 160) gcacatccctcgtccgaaa
(SEQ ID NO: 161) cacatccctcgtccgaaaa
(SEQ ID NO: 162) acatccctcgtccgaaaac
(SEQ ID NO: 163) catccctcgtccgaaaacg
(SEQ ID NO: 164) atccctcgtccgaaaacga
(SEQ ID NO: 165) tccctcgtccgaaaacgaa
(SEQ ID NO: 166) ccctcgtccgaaaacgaag
(SEQ ID NO: 167) cctcgtccgaaaacgaagg
(SEQ ID NO: 168) ctcgtccgaaaacgaaggt
(SEQ ID NO: 169) tcgtccgaaaacgaaggtt
(SEQ ID NO: 170) cgtccgaaaacgaaggttt
(SEQ ID NO: 171) gtccgaaaacgaaggtttg
(SEQ ID NO: 172) tccgaaaacgaaggtttgc
(SEQ ID NO: 173) ccgaaaacgaaggtttgca
(SEQ ID NO: 174) cgaaaacgaaggtttgcat
(SEQ ID NO: 175) gaaaacgaaggtttgcatc
(SEQ ID NO: 176) aaaacgaaggtttgcatcc
(SEQ ID NO: 177) aaacgaaggtttgcatcct
(SEQ ID NO: 178) aacgaaggtttgcatcctt
(SEQ ID NO: 179) acgaaggtttgcatccttg
(SEQ ID NO: 180) cgaaggtttgcatccttgg
(SEQ ID NO: 181) gaaggtttgcatccttggc
(SEQ ID NO: 182) aaggtttgcatccttggcg
(SEQ ID NO: 183) aggtttgcatccttggcga
(SEQ ID NO: 184) ggtttgcatccttggcgac
(SEQ ID NO: 185) gtttgcatccttggcgacc
(SEQ ID NO: 186) tttgcatccttggcgacca
(SEQ ID NO: 187) ttgcatccttggcgaccaa
(SEQ ID NO: 188) tgcatccttggcgaccaaa
(SEQ ID NO: 189) gcatccttggcgaccaaaa
(SEQ ID NO: 190) catccttggcgaccaaaaa
(SEQ ID NO: 191) atccttggcgaccaaaaac
(SEQ ID NO: 192) tccttggcgaccaaaaaca
(SEQ ID NO: 193) ccttggcgaccaaaaacat
(SEQ ID NO: 194) cttggcgaccaaaaacatt
(SEQ ID NO: 195) ttggcgaccaaaaacattg
(SEQ ID NO: 196) tggcgaccaaaaacattgt
(SEQ ID NO: 197) ggcgaccaaaaacattgtg
(SEQ ID NO: 198) gcgaccaaaaacattgtga
(SEQ ID NO: 199) cgaccaaaaacattgtgac
(SEQ ID NO: 200) gaccaaaaacattgtgacg
(SEQ ID NO: 201) accaaaaacattgtgacga
(SEQ ID NO: 202) ccaaaaacattgtgacgag
(SEQ ID NO: 203) caaaaacattgtgacgagg
(SEQ ID NO: 204) aaaaacattgtgacgaggc
(SEQ ID NO: 205) aaaacattgtgacgaggcc
(SEQ ID NO: 206) aaacattgtgacgaggcca
(SEQ ID NO: 207) aacattgtgacgaggccaa
(SEQ ID NO: 208) acattgtgacgaggccaat
(SEQ ID NO: 209) cattgtgacgaggccaatg
(SEQ ID NO: 210) attgtgacgaggccaatgc
(SEQ ID NO: 211) ttgtgacgaggccaatgcc
(SEQ ID NO: 212) tgtgacgaggccaatgcca
(SEQ ID NO: 213) gtgacgaggccaatgccaa
(SEQ ID NO: 214) tgacgaggccaatgccaac
(SEQ ID NO: 215) gacgaggccaatgccaacg
(SEQ ID NO: 216) acgaggccaatgccaacgg
(SEQ ID NO: 217) cgaggccaatgccaacgga
(SEQ ID NO: 218) gaggccaatgccaacggaa
(SEQ ID NO: 219) aggccaatgccaacggaat
(SEQ ID NO: 220) ggccaatgccaacggaatt
(SEQ ID NO: 221) gccaatgccaacggaattc
(SEQ ID NO: 222) ccaatgccaacggaattcc
(SEQ ID NO: 223) caatgccaacggaattcca
(SEQ ID NO: 224) aatgccaacggaattccat
(SEQ ID NO: 225) atgccaacggaattccatg
(SEQ ID NO: 226) tgccaacggaattccatgc
(SEQ ID NO: 227) gccaacggaattccatgca
(SEQ ID NO: 228) ccaacggaattccatgcat
(SEQ ID NO: 229) caacggaattccatgcatg
(SEQ ID NO: 230) aacggaattccatgcatga
(SEQ ID NO: 231) acggaattccatgcatgac
(SEQ ID NO: 232) cggaattccatgcatgaca
(SEQ ID NO: 233) ggaattccatgcatgacag
(SEQ ID NO: 234) gaattccatgcatgacagc
(SEQ ID NO: 235) aattccatgcatgacagcg
(SEQ ID NO: 236) attccatgcatgacagcgg
(SEQ ID NO: 237) ttccatgcatgacagcgga
(SEQ ID NO: 238) tccatgcatgacagcggac
(SEQ ID NO: 239) ccatgcatgacagcggacg
(SEQ ID NO: 240) catgcatgacagcggacga
(SEQ ID NO: 241) atgcatgacagcggacgac
(SEQ ID NO: 242) atgcatgacagcggacgac
(SEQ ID NO: 243) gcatgacagcggacgacct
(SEQ ID NO: 244) catgacagcggacgacctg
(SEQ ID NO: 245) atgacagcggacgacctga
(SEQ ID NO: 246) tgacagcggacgacctgaa
(SEQ ID NO: 247) gacagcggacgacctgaag
(SEQ ID NO: 248) acagcggacgacctgaaga
(SEQ ID NO: 249) cagcggacgacctgaagaa
(SEQ ID NO: 250) agcggacgacctgaagaag
(SEQ ID NO: 251) gcggacgacctgaagaagt
(SEQ ID NO: 252) cggacgacctgaagaagtt
(SEQ ID NO: 253) ggacgacctgaagaagttg
(SEQ ID NO: 254) gacgacctgaagaagttga
(SEQ ID NO: 255) acgacctgaagaagttgaa
(SEQ ID NO: 256) cgacctgaagaagttgaac
(SEQ ID NO: 257) gacctgaagaagttgaaca
(SEQ ID NO: 258) acctgaagaagttgaacaa
(SEQ ID NO: 259) cctgaagaagttgaacaag
(SEQ ID NO: 260) ctgaagaagttgaacaagg
(SEQ ID NO: 261) tgaagaagttgaacaagga
(SEQ ID NO: 262) gaagaagttgaacaaggac
(SEQ ID NO: 263) aagaagttgaacaaggaca
(SEQ ID NO: 264) agaagttgaacaaggacaa
(SEQ ID NO: 265) gaagttgaacaaggacaag
(SEQ ID NO: 266) aagttgaacaaggacaaga
(SEQ ID NO: 267) agttgaacaaggacaagaa
(SEQ ID NO: 268) gttgaacaaggacaagaag
(SEQ ID NO: 269) ttgaacaaggacaagaagc
(SEQ ID NO: 270) tgaacaaggacaagaagct
(SEQ ID NO: 271) gaacaaggacaagaagctg
(SEQ ID NO: 272) aacaaggacaagaagctga
(SEQ ID NO: 273) acaaggacaagaagctgat
(SEQ ID NO: 274) caaggacaagaagctgatc
(SEQ ID NO: 275) aaggacaagaagctgatct
(SEQ ID NO: 276) aggacaagaagctgatcta
(SEQ ID NO: 277) ggacaagaagctgatctaa
(SEQ ID NO: 278) gacaagaagctgatctaaa
(SEQ ID NO: 279) acaagaagctgatctaaaa
(SEQ ID NO: 280) caagaagctgatctaaaag
(SEQ ID NO: 281) aagaagctgatctaaaagc
(SEQ ID NO: 282) agaagctgatctaaaagct
(SEQ ID NO: 283) gaagctgatctaaaagctc
(SEQ ID NO: 284) aagctgatctaaaagctca
(SEQ ID NO: 285) agctgatctaaaagctcag
(SEQ ID NO: 286) gctgatctaaaagctcagc
(SEQ ID NO: 287) ctgatctaaaagctcagca
(SEQ ID NO: 288) tgatctaaaagctcagcaa
(SEQ ID NO: 289) gatctaaaagctcagcaaa
(SEQ ID NO: 290) atctaaaagctcagcaaaa
(SEQ ID NO: 291) tctaaaagctcagcaaaag
(SEQ ID NO: 292) ctaaaagctcagcaaaagc
(SEQ ID NO: 293) taaaagctcagcaaaagct
(SEQ ID NO: 294) aaaagctcagcaaaagcta
(SEQ ID NO: 295) aaagctcagcaaaagctac
(SEQ ID NO: 296) aagctcagcaaaagctacc
(SEQ ID NO: 297) agctcagcaaaagctacca
(SEQ ID NO: 298) gctcagcaaaagctaccac
(SEQ ID NO: 299) ctcagcaaaagctaccacg
(SEQ ID NO: 300) tcagcaaaagctaccacgc
(SEQ ID NO: 301) cagcaaaagctaccacgct
(SEQ ID NO; 302) agcaaaagctaccacgctt
(SEQ ID NO: 303) gcaaaagctaccacgcttt
(SEQ ID NO: 304) caaaagctaccacgatttc
(SEQ ID NO: 305) aaaagctaccacgctttcc
(SEQ ID NO: 306) aaagctaccacgctttcct
(SEQ ID NO: 307) aagctaccacgctttcctt
(SEQ ID NO: 308) agctaccacgctttccttg
(SEQ ID NO: 309) gctaccacgctttccttgc
(SEQ ID NO: 310) ctaccacgctttccttgcc
(SEQ ID NO: 311) taccacgctttccttgcct
(SEQ ID NO: 312) accacgctttccttgcctt
(SEQ ID NO: 313) ccacgctttccttgccttc
(SEQ ID NO: 314) cacgctttccttgccttcg
(SEQ ID NO: 315) acgctttccttgccttcga
(SEQ ID NO: 316) cgctttccttgccttcgaa
(SEQ ID NO: 317) gctttccttgccttcgaat
(SEQ ID NO: 318) ctttccttgccttcgaatc
(SEQ ID NO: 319) tttccttgccttcgaatca
(SEQ ID NO: 320) ttccttgccttcgaatcac
(SEQ ID NO: 321) tccttgccttcgaatcact
(SEQ ID NO: 322) ccttgccttcgaatcactc
(SEQ ID NO: 323) cttgccttcgaatcactca
(SEQ ID NO: 324) ttgccttcgaatcactcat
(SEQ ID NO: 325) tgccttcgaatcactcatc
(SEQ ID NO: 326) gccttcgaatcactcatca
(SEQ ID NO: 327) ccttcgaatcactcatcaa
(SEQ ID NO: 328) cttcgaatcactcatcaaa
(SEQ ID NO: 329) ttcgaatcactcatcaaac
(SEQ ID NO: 330) tcgaatcactcatcaaaca
(SEQ ID NO: 331) cgaatcactcatcaaacaa
(SEQ ID NO: 332) gaatcactcatcaaacaaa
(SEQ ID NO: 333) aatcactcatcaaacaaat
(SEQ ID NO: 334) atcactcatcaaacaaatc
(SEQ ID NO: 335) tcactcatcaaacaaatcc
(SEQ ID NO: 336) cactcatcaaacaaatccc
(SEQ ID NO: 337) actcatcaaacaaatccct
(SEQ ID NO: 338) ctcatcaaacaaatccctc
(SEQ ID NO: 339) tcatcaaacaaatccctcg
(SEQ ID NO: 340) catcaaacaaatccctcgt
(SEQ ID NO: 341) atcaaacaaatccctcgta
(SEQ ID NO: 342) tcaaacaaatccctcgtat
(SEQ ID NO: 343) caaacaaatccctcgtatt
(SEQ ID NO: 344) aaacaaatccctcgtattc
(SEQ ID NO: 345) aacaaatccctcgtattct
(SEQ ID NO: 346) acaaatccctcgtattctt
(SEQ ID NO: 347) caaatccctcgtaftcttg
(SEQ ID NO: 348) aaatccctcgtattcttgg
(SEQ ID NO: 349) aatccctcgtattcttggt
(SEQ ID NO: 350) atccctcgtattcttggtc
(SEQ ID NO: 351) tccctcgtattcttggtcc
(SEQ ID NO: 352) ccctcgtattcttggtccc
(SEQ ID NO: 353) cctcgtattcttggtcccg
(SEQ ID NO: 354) ctcgtattcttggtcccgg
(SEQ ID NO: 355) tcgtattcttggtcccgga
(SEQ ID NO: 356) cgtattcttggtcccggac
(SEQ ID NO: 357) gtattcttggtcccggact
(SEQ ID NO: 358) tattcttggtcccggactg
(SEQ ID NO: 359) attcttggtcccggactga
(SEQ ID NO: 360) ttcttggtccoggactgaa
(SEQ ID NO: 361) tcttggtcccggactgaac
(SEQ ID NO: 362) cttggtcccggactgaaca
(SEQ ID NO: 363) ttggtcccggactgaacaa
(SEQ ID NO: 364) tggtcccggactgaacaag
(SEQ ID NO: 365) ggtcccggactgaacaagg
(SEQ ID NO: 366) gtcccggactgaacaaggc
(SEQ ID NO: 367) tcccggactgaacaaggct
(SEQ ID NO: 368) cccggactgaacaaggctg
(SEQ ID NO: 369) ccggactgaacaaggctgg
(SEQ ID NO: 370) cggactgaacaaggctggc
(SEQ ID NO: 371) ggactgaacaaggctggca
(SEQ ID NO: 372) gactgaacaaggctggcaa
(SEQ ID NO: 373) actgaacaaggctggcaag
(SEQ ID NO: 374) ctgaacaaggctggcaagt
(SEQ ID NO: 375) tgaacaaggctggcaagtt
(SEQ ID NO: 376) gaacaaggctggcaagttc
(SEQ ID NO: 377) aacaaggctggcaagttcc
(SEQ ID NO: 378) acaaggctggcaagttccc
(SEQ ID NO: 379) caaggctggcaagttccca
(SEQ ID NO: 380) aaggctggcaagttcccaa
(SEQ ID NO: 381) aggctggcaagttcccaag
(SEQ ID NO: 382) ggctggcaagttcccaagt
(SEQ ID NO: 383) gctggcaagttcccaagtg
(SEQ ID NO: 384) ctggcaagttcccaagtgt
(SEQ ID NO: 385) tggcaagttcccaagtgtg
(SEQ ID NO: 386) ggcaagttcccaagtgtgg
(SEQ ID NO: 387) gcaagttcccaagtgtggt
(SEQ ID NO: 388) caagttcccaagtgtggtg
(SEQ ID NO: 389) aagttcccaagtgtggtgt
(SEQ ID NO: 390) agttcccaagtgtggtgtc
(SEQ ID NO: 391) gttcccaagtgtggtgtca
(SEQ ID NO: 392) ttcccaagtgtggtgtcac
(SEQ ID NO: 393) tcccaagtgtggtgtcaca
(SEQ ID NO: 394) cccaagtgtggtgtcacac
(SEQ ID NO: 395) ccaagtgtggtgtcacaca
(SEQ ID NO: 396) caagtgtggtgtcacacaa
(SEQ ID NO: 397) aagtgtggtgtcacacaac
(SEQ ID NO: 398) agtgtggtgtcacacaacg
(SEQ ID NO: 399) gtgtggtgtcacacaacga
(SEQ ID NO: 400) tgtggtgtcacacaacgac
(SEQ ID NO: 401) gtggtgtcacacaacgaca
(SEQ ID NO: 402) tggtgtcacacaacgacat
(SEQ ID NO: 403) ggtgtcacacaacgacatg
(SEQ ID NO: 404) gtgtcacacaacgacatgc
(SEQ ID NO: 405) tgtcacacaacgacatgct
(SEQ ID NO: 406) gtcacacaacgacatgctg
(SEQ ID NO: 407) tcacacaacgacatgctga
(SEQ ID NO: 408) cacacaacgacatgctgaa
(SEQ ID NO: 409) acacaacgacatgctgaac
(SEQ ID NO: 410) cacaacgacatgctgaacg
(SEQ ID NO: 411) acaacgacatgctgaacgc
(SEQ ID NO: 412) caacgacatgctgaacgca
(SEQ ID NO: 413) aacgacatgctgaacgcaa
(SEQ ID NO: 414) acgacatgctgaacgcaaa
(SEQ ID NO: 415) cgacatgctgaacgcaaag
(SEQ ID NO: 416) gacatgctgaacgcaaagg
(SEQ ID NO: 417) acatgctgaacgcaaaggt
(SEQ ID NO: 418) catgctgaacgcaaaggtg
(SEQ ID NO: 419) atgctgaacgcaaaggtgg
(SEQ ID NO: 420) tgctgaacgcaaaggtgga
(SEQ ID NO: 421) gctgaacgcaaaggtggat
(SEQ ID NO: 422) ctgaacgcaaaggtggatg
(SEQ ID NO: 423) tgaacgcaaaggtggatga
(SEQ ID NO: 424) gaacgcaaaggtggatgaa
(SEQ ID NO: 425) aacgcaaaggtggatgaag
(SEQ ID NO: 426) acgcaaaggtggatgaagt
(SEQ ID NO: 427) cgcaaaggtggatgaagtg
(SEQ ID NO: 428) gcaaaggtggatgaagtga
(SEQ ID NO: 429) caaaggtggatgaagtgaa
(SEQ ID NO: 430) aaaggtggatgaagtgaag
(SEQ ID NO: 431) aaggtggatgaagtgaagg
(SEQ ID NO: 432) aggtggatgaagtgaaggc
(SEQ ID NO: 433) ggtggatgaagtgaaggcg
(SEQ ID NO: 434) gtggatgaagtgaaggcga
(SEQ ID NO: 435) tggatgaagtgaaggcgaa
(SEQ ID NO: 436) ggatgaagtgaaggcgaac
(SEQ ID NO: 437) gatgaagtgaaggcgaacc
(SEQ ID NO: 438) atgaagtgaaggcgaaccg
(SEQ ID NO: 439) tgaagtgaaggcgaaccgc
(SEQ ID NO: 440) gaagtgaaggcgaaccgca
(SEQ ID NO: 441) aagtgaaggcgaaccgcaa
(SEQ ID NO: 442) agtgaaggcgaaccgcaaa
(SEQ ID NO: 443) gtgaaggcgaaccgcaaat
(SEQ ID NO: 444) tgaaggcgaaccgcaaatt
(SEQ ID NO: 445) gaaggcgaaccgcaaattc
(SEQ ID NO: 446) aaggcgaaccgcaaattcg
(SEQ ID NO: 447) aggcgaaccgcaaattcga
(SEQ ID NO: 448) ggegaaccgcaaattcgaa
(SEQ ID NO: 449) gcgaaccgcaaattcgaaa
(SEQ ID NO: 450) cgaaccgcaaattcgaaat
(SEQ ID NO: 451) gaaccgcaaattcgaaatg
(SEQ ID NO: 452) aaccgcaaattcgaaatga
(SEQ ID NO: 453) accgcaaattcgaaatgaa
(SEQ ID NO: 454) ccgcaaattcgaaatgaaa
(SEQ ID NO: 455) cgcaaattcgaaatgaaac
(SEQ ID NO: 456) gcaaattcgaaatgaaaca
(SEQ ID NO: 457) caaattcgaaatgaaacag
(SEQ ID NO: 458) aaattcgaaatgaaacagg
(SEQ ID NO: 459) aattcgaaatgaaacaggt
(SEQ ID NO: 460) attcgaaatgaaacaggtg
(SEQ ID NO: 461) ttcgaaatgaaacaggtgc
(SEQ ID NO: 462) tcgaaatgaaacaggtgct
(SEQ ID NO: 463) cgaaatgaaacaggtgctc

TABLE 2
Antisense siRNA sequences to hg-rps-23

(SEQ ID NO: 464) gagcaccuguuucauuucg
(SEQ ID NO: 465) agcaccuguuucauuucga
(SEQ ID NO: 466) gcaccuguuucauuucgaa
(SEQ ID NO: 467) caccuguuucauuucgaau
(SEQ ID NO: 468) accuguuucauuucgaauu
(SEQ ID NO: 469) ccuguuucauuucgaauuu
(SEQ ID NO: 470) cuguuucauuucgaauuug
(SEQ ID NO: 471) uguuucauuucgaauuugc
(SEQ ID NO: 472) guuucauuucgaauuugcg
(SEQ ID NO: 473) uuucauuucgaauuugcgg
(SEQ ID NO: 474) uucauuucgaauuugcggu
(SEQ ID NO: 475) ucauuucgaauuugcgguu
(SEQ ID NO: 476) cauuucgaauuugcgguuc
(SEQ ID NO: 477) auuucgaauuugcgguucg
(SEQ ID NO: 478) uuucgaauuugcgguucgc
(SEQ ID NO: 479) uucgaauuugcgguucgcc
(SEQ ID NO: 480) ucgaauuugcgguucgccu
(SEQ ID NO: 481) cgaauuugcgguucgccuu
(SEQ ID NO: 482) gaauuugcgguucgccuuc
(SEQ ID NO: 483) aauuugcgguucgccuuca
(SEQ ID NO: 484) auuugcgguucgccuucac
(SEQ ID NO: 485) uuugcgguucgccuucacu
(SEQ ID NO: 486) uugcgguucgccuucacuu
(SEQ ID NO: 487) ugcgguucgccuucacuuc
(SEQ ID NO: 488) gcgguucgccuucacuuca
(SEQ ID NO: 489) cgguucgccuucacuucau
(SEQ ID NO: 490) gguucgccuucacuucauc
(SEQ ID NO: 491) guucgccuucacuucaucc
(SEQ ID NO: 492) uucgccuucacuucaucca
(SEQ ID NO: 493) ucgccuucacuucauccac
(SEQ ID NO: 494) cgccuucacuucauccacc
(SEQ ID NO: 495) gccuucacuucauccaccu
(SEQ ID NO: 496) ccuucacuucauccaccuu
(SEQ ID NO: 497) cuucacuucauccaccuuu
(SEQ ID NO: 498) uucacuucauccaccuuug
(SEQ ID NO: 499) ucacuucauccaccuuugc
(SEQ ID NO: 500) cacuucauccaccuuugcg
(SEQ ID NO: 501) acuucauccaccuuugcgu
(SEQ ID NO: 502) cuucauccaccuuugcguu
(SEQ ID NO: 503) uucauccaccuuugcguuc
(SEQ ID NO: 504) ucauccaccuuugcguuca
(SEQ ID NO: 505) cauccaccuuugcguucag
(SEQ ID NO: 506) auccaccuuugcguucagc
(SEQ ID NO: 507) uccaccuuugcguucagca
(SEQ ID NO: 508) ccaccuuugcguucagcau
(SEQ ID NO: 509) caccuuugcguucagcaug
(SEQ ID NO: 510) accuuugcguucagcaugu
(SEQ ID NO: 511) ccuuugcguucagcauguc
(SEQ ID NO: 512) cuuugcguucagcaugucg
(SEQ ID NO: 513) uuugcguucagcaugucgu
(SEQ ID NO: 514) uugcguucagcaugucguu
(SEQ ID NO: 515) ugcguucagcaugucguug
(SEQ ID NO: 516) gcguucagcaugucguugu
(SEQ ID NO: 517) cguucagcaugucguugug
(SEQ ID NO: 518) guucagcaugucguugugu
(SEQ ID NO: 519) uucagcaugucguugugug
(SEQ ID NO: 520) ucagcaugucguuguguga
(SEQ ID NO: 521) cagcaugucguugugugac
(SEQ ID NO: 522) agcaugucguugugugaca
(SEQ ID NO: 523) gcaugucguugugugacac
(SEQ ID NO: 524) caugucguugugugacacc
(SEQ ID NO: 525) augucguugugugacacca
(SEQ ID NO: 526) ugucguugugugacaccac
(SEQ ID NO: 527) gucguugugugacaccaca
SEQ ID NO: 528) ucguugugugacaccacac
(SEQ ID NO: 529) cguugugugacaccacacu
(SEQ ID NO: 530) guugugugacaccacacuu
(SEQ ID NO: 531) uugugugacaccacacuug
(SEQ ID NO: 532) ugugugacaccacacuugg
(SEQ ID NO: 533) gugugacaccacacuuggg
(SEQ ID NO: 534) ugugacaccacacuuggga
(SEQ ID NO: 535) gugacaccacacuugggaa
(SEQ ID NO: 536) ugacaccacacuugggaac
(SEQ ID NO: 537) gacaccacacuugggaacu
(SEQ ID NO: 538) acaccacacuugggaacuu
(SEQ ID NO: 539) caccacacuugggaacuug
(SEQ ID NO: 540) accacacuugggaacuugc
(SEQ ID NO: 541) ccacacuugggaacuugcc
(SEQ ID NO: 542) cacacuugggaacuugcca
(SEQ ID NO: 543) acacuugggaacuugccag
(SEQ ID NO: 544) cacuugggaacuugccagc
(SEQ ID NO: 545) acuugggaacuugccagcc
(SEQ ID NO: 546) cuugggaacuugccagccu
(SEQ ID NO: 547) uugggaacuugccagccuu
(SEQ ID NO: 548) ugggaacuugccagccuug
(SEQ ID NO: 549) gggaacuugccagccuugu
(SEQ ID NO: 550) ggaacuugccagccuuguu
(SEQ ID NO: 551) gaacuugccagccuuguuc
(SEQ ID NO: 552) aacuugccagccuuguuca
(SEQ ID NO: 553) acuugccagccuuguucag
(SEQ ID NO: 554) cuugccagccuuguucagu
(SEQ ID NO: 555) uugccagccuuguucaguc
(SEQ ID NO: 556) ugccagccuuguucagucc
(SEQ ID NO: 557) gccagccuuguucaguccg
(SEQ ID NO: 558) ccagccuuguucaguccgg
(SEQ ID NO: 559) cagccuuguucaguccggg
(SEQ ID NO: 560) agccuuguucaguccggga
(SEQ ID NO: 561) gccuuguucaguccgggac
(SEQ ID NO: 562) ccuuguucaguccgggacc
(SEQ ID NO: 563) cuuguucaguccgggacca
(SEQ ID NO: 564) uuguucaguccgggaccaa
(SEQ ID NO: 565) uguucaguccgggaccaag
(SEQ ID NO: 566) guucaguccgggaccaaga
(SEQ ID NO: 567) uucaguccgggaccaagaa
(SEQ ID NO: 568) ucaguccgggaccaagaau
(SEQ ID NO: 569) caguccgggaccaagaaua
(SEQ ID NO: 570) aguccgggaccaagaauac
(SEQ ID NO: 571) guccgggaccaagaauacg
(SEQ ID NO: 572) uccgggaccaagaauacga
(SEQ ID NO: 573) ccgggaccaagaauacgag
(SEQ ID NO: 574) cgggaccaagaauacgagg
(SEQ ID NO: 575) gggaccaagaauacgaggg
(SEQ ID NO: 576) ggaccaagaauacgaggga
(SEQ ID NO: 577) gaccaagaauacgagggau
(SEQ ID NO: 578) accaagaauacgagggauu
(SEQ ID NO: 579) ccaagaauacgagggauuu
(SEQ ID NO: 580) caagaauacgagggauuug
(SEQ ID NO: 581) aagaauacgagggauuugu
(SEQ ID NO: 582) agaauacgagggauuuguu
(SEQ ID NO: 583) gaauacgagggauuuguuu
(SEQ ID NO: 584) aauacgagggauuuguuug
(SEQ ID NO: 585) auacgagggauuuguuuga
(SEQ ID NO: 586) uacgagggauuuguuugau
(SEQ ID NO: 587) acgagggauuuguuugaug
(SEQ ID NO: 588) cgagggauuuguuugauga
(SEQ ID NO: 589) gagggauuuguuugaugag
(SEQ ID NO: 590) agggauuuguuugaugagu
(SEQ ID NO: 591) gggauuuguuugaugagug
(SEQ ID NO: 592) ggauuuguuugaugaguga
(SEQ ID NQ: 593) gauuuguuugaugagugau
(SEQ ID NO: 594) auuuguuugaugagugauu
(SEQ ID NO: 595) uuuguuugaugagugauuc
(SEQ ID NO: 596) uuguuugaugagugauucg
(SEQ ID NO: 597) uguuugaugagugauucga
(SEQ ID NO: 598) guuugaugagugauucgaa
(SEQ ID NO: 599) uuugaugagugauucgaag
(SEQ ID NO: 600) uugaugagugauucgaagg
(SEQ ID NO: 601) ugaugagugauucgaaggc
(SEQ ID NO: 602) gaugagugauucgaaggca
(SEQ ID NO: 603) augagugauucgaaggcaa
(SEQ ID NO: 604) ugagugauucgaaggcaag
(SEQ ID NO: 605) gagugauucgaaggcaagg
(SEQ ID NO: 606) agugauucgaaggcaagga
(SEQ ID NO: 607) gugauucgaaggcaaggaa
(SEQ ID NO: 608) ugauucgaaggcaaggaaa
(SEQ ID NO: 609) gauucgaaggcaaggaaag
(SEQ ID NO: 610) auucgaaggcaaggaaagc
(SEQ ID NO: 611) uucgaaggcaaggaaagcg
(SEQ ID NO: 612) ucgaaggcaaggaaagcgu
(SEQ ID NO: 613) cgaaggcaaggaaagcgug
(SEQ ID NO: 614) gaaggcaaggaaagcgugg
(SEQ ID NO: 615) aaggcaaggaaagcguggu
(SEQ ID NO: 616) aggcaaggaaagcguggua
(SEQ ID NO: 617) ggcaaggaaagcgugguag
(SEQ ID NO: 618) gcaaggaaagcgugguagc
(SEQ ID NO: 619) caaggaaagcgugguagcu
(SEQ ID NO: 620) aaggaaagcgugguagcuu
(SEQ ID NO: 621) aggaaagcgugguagcuuu
(SEQ ID NO: 622) ggaaagcgugguagcuuuu
(SEQ ID NO: 623) gaaagcgugguagcuuuug
(SEQ ID NO: 624) aaagcgugguagcuuuugc
(SEQ ID NO: 625) aagcgugguagcuuuugcu
(SEQ ID NO: 626) agcgugguagcuuuugcug
(SEQ ID NO: 627) gcgugguagcuuuugcuga
(SEQ ID NO: 628) cgugguagcuuuugcugag
(SEQ ID NO: 629) gugguagcuuuugcugagc
(SEQ ID NO: 630) ugguagcuuuugcugagcu
(SEQ ID NO: 631) gguagcuuuugcugagcuu
(SEQ ID NO: 632) guagcuuuugcugagcuuu
(SEQ ID NO: 633) uagcuuuugcugagcuuuu
(SEQ ID NO: 634) agcuuuugcugagcuuuua
(SEQ ID NO: 635) gcuuuugcugagcuuuuag
(SEQ ID NO: 636) cuuuugcugagcuuuuaga
(SEQ ID NO: 637) uuuugcugagcuuuuagau
(SEQ ID NO: 638) uuugcugagcuuuuagauc
(SEQ ID NO: 639) uugcugagcuuuuagauca
(SEQ ID NO: 640) ugcugagcuuuuagaucag
(SEQ ID NO: 641) gcugagcuuuuagaucagc
(SEQ ID NO: 642) cugagcuuuuagaucagcu
(SEQ ID NO: 643) ugagcuuuuagaucagcuu
(SEQ ID NO: 644) gagcuuuuagaucagcuuc
(SEQ ID NO: 645) agcuuuuagaucagcuucu
(SEQ ID NO: 646) gcuuuuagaucagcuucuu
(SEQ ID NO: 647) cuuuuagaucagcuucuug
(SEQ ID NO: 648) uuuuagaucagcuucuugu
(SEQ ID NO: 649) uuuagaucagcuucuuguc
(SEQ ID NO: 650) uuagaucagcuucuugucc
(SEQ ID NO: 651) uagaucagcuucuuguccu
(SEQ ID NO: 652) agaucagcuucuuguccuu
(SEQ ID NO: 653) gaucagcuucuuguccuug
(SEQ ID NO: 654) aucagcuucuuguccuugu
(SEQ ID NO: 655) ucagcuucuuguccuuguu
(SEQ ID NO: 656) cagcuucuuguccuuguuc
(SEQ ID NO: 657) agcuucuuguccuuguuca
(SEQ ID NO: 658) gcuucuuguccuuguucaa
(SEQ ID NO: 659) cuucuuguccuuguucaac
(SEQ ID NO: 660) uucuuguccuuguucaacu
(SEQ ID NO: 661) ucuuguccuuguucaacuu
(SEQ ID NO: 662) cuuguccuuguucaacuuc
(SEQ ID NO: 663) uuguccuuguucaacuucu
(SEQ ID NO: 664) uguccuuguucaacuucuu
(SEQ ID NO: 665) guccuuguucaacuucuuc
(SEQ ID NO: 666) uccuuguucaacuucuuca
(SEQ ID NO: 667) ccuuguucaacuucuucag
(SEQ ID NO: 668) cuuguucaacuucuucagg
(SEQ ID NO: 669) uuguucaacuucuucaggu
(SEQ ID NO: 670) uguucaacuucuucagguc
(SEQ ID NO: 671) guucaacuucuucaggucg
(SEQ ID NO: 672) uucaacuucuucaggucgu
(SEQ ID NO: 673) ucaacuucuucaggucguc
(SEQ ID NO: 674) caacuucuucaggucgucc
(SEQ ID NO: 675) aacuucuucaggucguccg
(SEQ ID NO: 676) acuucuucaggucguccgc
(SEQ ID NO: 677) cuucuucaggucguccgcu
(SEQ ID NO: 678) uucuucaggucguccgcug
(SEQ ID NO: 679) ucuucaggucguccgcugu
(SEQ ID NO: 680) cuucaggucguccgcuguc
(SEQ ID NO: 681) uucaggucguccgcuguca
(SEQ ID NO: 682) ucaggucguccgcugucau
(SEQ ID NO: 683) caggucguccgcugucaug
(SEQ ID NO: 684) aggucguccgcugucaugc
(SEQ ID NO: 685) gucguccgcugucaugcau
(SEQ ID NO: 686) gucguccgcugucaugcau
(SEQ ID NO: 687) ucguccgcugucaugcaug
(SEQ ID NO: 688) cguccgcugucaugcaugg
(SEQ ID NO: 689) guccgcugucaugcaugga
(SEQ ID NO: 690) uccgcugucaugcauggaa
(SEQ ID NO: 691) ccgcugucaugcauggaau
(SEQ ID NO: 692) cgcugucaugcauggaauu
(SEQ ID NO: 693) gcugucaugcauggaauuc
(SEQ ID NO: 694) cugucaugcauggaauucc
(SEQ ID NO: 695) ugucaugcauggaauuccg
(SEQ ID NO: 696) gucaugcauggaauuccgu
(SEQ ID NO: 697) ucaugcauggaauuccguu
(SEQ ID NO: 698) caugcauggaauuccguug
(SEQ ID NO: 699) augcauggaauuccguugg
(SEQ ID NO: 700) ugcauggaauuccguuggc
(SEQ ID NO: 701) gcauggaauuccguuggca
(SEQ ID NO: 702) cauggaauuccguuggcau
(SEQ ID NO: 703) auggaauuccguuggcauu
(SEQ ID NO: 704) uggaauuccguuggcauug
(SEQ ID NO: 705) ggaauuccguuggcauugg
(SEQ ID NO: 706) gaauuccguuggcauuggc
(SEQ ID NO: 707) aauuccguuggcauuggcc
(SEQ ID NO: 708) auuccguuggcauuggccu
(SEQ ID NO: 709) uuccguuggcauuggccuc
(SEQ ID NO: 710) uccguuggcauuggccucg
(SEQ ID NO: 711) ccguuggcauuggccucgu
(SEQ ID NO: 712) cguuggcauuggccucguc
(SEQ ID NO: 713) guuggcauuggccucguca
(SEQ ID NO: 714) uuggcauuggccucgucac
(SEQ ID NO: 715) uggcauuggccucgucaca
(SEQ ID NO: 716) ggcauuggccucgucacaa
(SEQ ID NO: 717) gcauuggccucgucacaau
(SEQ ID NO: 718) cauuggccucgucacaaug
(SEQ ID NO: 719) auuggccucgucacaaugu
(SEQ ID NO: 720) uuggccucgucacaauguu
(SEQ ID NO: 721) uggccucgucacaauguuu
(SEQ ID NO: 722) ggccucgucacaauguuuu
(SEQ ID NO: 723) gccucgucacaauguuuuu
(SEQ ID NO: 724) ccucgucacaauguuuuug
(SEQ ID NO: 725) cucgucacaauguuuuugg
(SEQ ID NO: 726) ucgucacaauguuuuuggu
(SEQ ID NO: 727) cgucacaauguuuuugguc
(SEQ ID NO: 728) gucacaauguuuuuggucg
(SEQ ID NO: 729) ucacaauguuuuuggucgc
(SEQ ID NO: 730) cacaauguuuuuggucgcc
(SEQ ID NO: 731) acaauguuuuuggucgcca
(SEQ ID NO: 732) caauguuuuuggucgccaa
(SEQ ID NO: 733) aauguuuuuggucgccaag
(SEQ ID NO: 734) auguuuuuggucgccaagg
(SEQ ID NO: 735) uguuuuuggucgccaagga
(SEQ ID NO: 736) guuuuuggucgccaaggau
(SEQ ID NO: 737) uuuuuggucgccaaggaug
(SEQ ID NO: 738) uuuuggucgccaaggaugc
(SEQ ID NO: 739) uuuggucgccaaggaugca
(SEQ ID NO: 740) uuggucgccaaggaugcaa
(SEQ ID NO: 741) uggucgccaaggaugcaaa
(SEQ ID NO: 742) ggucgccaaggaugcaaac
(SEQ ID NO: 743) gucgccaaggaugcaaacc
(SEQ ID NO: 744) ucgccaaggaugcaaaccu
(SEQ ID NO: 745) cgccaaggaugcaaaccuu
(SEQ ID NO: 746) gccaaggaugcaaaccuuc
(SEQ ID NO: 747) ccaaggaugcaaaccuucg
(SEQ ID NO: 748) caaggaugcaaaccuucgu
(SEQ ID NO: 749) aaggaugcaaaccuucguu
(SEQ ID NO: 750) aggaugcaaaccuucguuu
(SEQ ID NO: 751) ggaugcaaaccuucguuuu
(SEQ ID NO: 752) gaugcaaaccuucguuuuc
(SEQ ID NO: 753) augcaaaccuucguuuucg
(SEQ ID NO: 754) ugcaaaccuucguuuucgg
(SEQ ID NO: 755) gcaaaccuucguuuucgga
(SEQ ID NO: 756) caaaccuucguuuucggac
(SEQ ID NO: 757) aaaccuucguuuucggacg
(SEQ ID NO: 758) aaccuucguuuucggacga
(SEQ ID NO: 759) accuucguuuucggacgag
(SEQ ID NO: 760) ccuucguuuucggacgagg
(SEQ ID NO: 761) cuucguuuucggacgaggg
(SEQ ID NO: 762) uucguuuucggacgaggga
(SEQ ID NO: 763) ucguuuucggacgagggau
(SEQ ID NO: 764) cguuuucggacgagggaug
(SEQ ID NO: 765) guuuucggacgagggaugu
(SEQ ID NO: 766) uuuucggacgagggaugug
(SEQ ID NO: 767) uuucggacgagggaugugc
(SEQ ID NO: 768) uucggacgagggaugugcu
(SEQ ID NO: 769) ucggacgagggaugugcuu
(SEQ ID NO: 770) cggacgagggaugugcuuc
(SEQ ID NO: 771) ggacgagggaugugcuuca
(SEQ ID NO: 772) gacgagggaugugcuucag
(SEQ ID NO: 773) acgagggaugugcuucagu
(SEQ ID NO: 774) cgagggaugugcuucaguc
(SEQ ID NO: 775) gagggaugugcuucagucu
(SEQ ID NO: 776) agggaugugcuucagucua
(SEQ ID NO: 777) gggaugugcuucagucuaa
(SEQ ID NO: 778) ggaugugcuucagucuaac
(SEQ ID NO: 779) gaugugcuucagucuaaca
(SEQ ID NO: 780) augugcuucagucuaacag
(SEQ ID NO: 781) ugugcuucagucuaacagu
(SEQ ID NO: 782) gugcuucagucuaacaguu
(SEQ ID NO: 783) ugcuucagucuaacaguuc
(SEQ ID NO: 784) gcuucagucuaacaguucc
(SEQ ID NO: 785) cuucagucuaacaguucca
(SEQ ID NO: 786) uucagucuaacaguuccac
(SEQ ID NO: 787) ucagucuaacaguuccacu
(SEQ ID NO: 788) cagucuaacaguuccacug
(SEQ ID NO: 789) agucuaacaguuccacuga
(SEQ ID NO: 790) gucuaacaguuccacugaa
(SEQ ID NO: 791) ucuaacaguuccacugaaa
(SEQ ID NO: 792) cuaacaguuccacugaaac
(SEQ ID NO: 793) uaacaguuccacugaaacg
(SEQ ID NO: 794) aacaguuccacugaaacgc
(SEQ ID NO: 795) acaguuccacugaaacgcu
(SEQ ID NO: 796) caguuccacugaaacgcuu
(SEQ ID NO: 797) aguuccacugaaacgcuug
(SEQ ID NO: 798) guuccacugaaacgcuugu
(SEQ ID NO: 799) uuccacugaaacgcuuguc
(SEQ ID NO: 800) uccacugaaacgcuugucc
(SEQ ID NO: 801) ccacugaaacgcuuguccu
(SEQ ID NO: 802) cacugaaacgcuuguccuu
(SEQ ID NO: 803) acugaaacgcuuguccuuc
(SEQ ID NO: 804) cugaaacgcuuguccuucu
(SEQ ID NO: 805) ugaaacgcuuguccuucug
(SEQ ID NO: 806) gaaacgcuuguccuucugu
(SEQ ID NO: 807) aaacgcuuguccuucugug
(SEQ ID NO: 808) aacgcuuguccuucugugg
(SEQ ID NO: 809) acgcuuguccuucuguggg
(SEQ ID NO: 810) cgcuuguccuucugugggu
(SEQ ID NO: 811) gcuuguccuucuguggguc
(SEQ ID NO: 812) cuuguccuucugugggucg
(SEQ ID NO: 813) uuguccuucugugggucgu
(SEQ ID NO: 814) uguccuucugugggucgua
(SEQ ID NO: 815) guccuucugugggucguag
(SEQ ID NO: 816) uccuucugugggucguagu
(SEQ ID NO: 817) ccuucugugggucguaguu
(SEQ ID NO: 818) cuucugugggucguaguuu
(SEQ ID NO: 819) uucugugggucguaguuuu
(SEQ ID NO: 820) ucugugggucguaguuuuu
(SEQ ID NO: 821) cugugggucguaguuuuuc
(SEQ ID NO: 822) ugugggucguaguuuuuca
(SEQ ID NO: 823) gugggucguaguuuuucag
(SEQ ID NO: 824) ugggucguaguuuuucaga
(SEQ ID NO: 825) gggucguaguuuuucagag
(SEQ ID NO: 826) ggucguaguuuuucagagc
(SEQ ID NO: 827) gucguaguuuuucagagca
(SEQ ID NO: 828) ucguaguuuuucagagcaa
(SEQ ID NO: 829) cguaguuuuucagagcaau
(SEQ ID NO: 830) guaguuuuucagagcaauu
(SEQ ID NO: 831) uaguuuuucagagcaauuu
(SEQ ID NO: 832) aguuuuucagagcaauuug
(SEQ ID NO: 833) guuuuucagagcaauuugc
(SEQ ID NO: 834) uuuuucagagcaauuugca
(SEQ ID NO: 835) uuuucagagcaauuugcaa
(SEQ ID NO: 836) uuucagagcaauuugcaau
(SEQ ID NO: 837) uucagagcaauuugcaauu
(SEQ ID NO: 838) ucagagcaauuugcaauuc
(SEQ ID NO: 839) cagagcaauuugcaauuca
(SEQ ID NO: 840) agagcaauuugcaauucaa
(SEQ ID NO: 841) gagcaauuugcaauucaau
(SEQ ID NO: 842) agcaauuugcaauucaauc
(SEQ ID NO: 843) gcaauuugcaauucaaucg
(SEQ ID NO: 844) caauuugcaauucaaucgu
(SEQ ID NO: 845) aauuugcaauucaaucguu
(SEQ ID NO: 846) auuugcaauucaaucguuu
(SEQ ID NO: 847) uuugcaauucaaucguuuc
(SEQ ID NO: 848) uugcaauucaaucguuucu
(SEQ ID NO: 849) ugcaauucaaucguuucuc
(SEQ ID NO: 850) gcaauucaaucguuucucg
(SEQ ID NO: 851) caauucaaucguuucucgg
(SEQ ID NO: 852) aauucaaucguuucucgga
(SEQ ID NO: 853) auucaaucguuucucggaa
(SEQ ID NO: 854) uucaaucguuucucggaaa
(SEQ ID NO: 855) ucaaucguuucucggaaau
(SEQ ID NO: 856) caaucguuucucggaaauu
(SEQ ID NO: 857) aaucguuucucggaaauug
(SEQ ID NO: 858) aucguuucucggaaauugc
(SEQ ID NO: 859) ucguuucucggaaauugcg
(SEQ ID NO: 860) cguuucucggaaauugcgc
(SEQ ID NO: 861) guuucucggaaauugcgcu
(SEQ ID NO: 862) uuucucggaaauugcgcuu
(SEQ ID NO: 863) uucucggaaauugcgcuuc
(SEQ ID NO: 864) ucucggaaauugcgcuucu
(SEQ ID NO: 865) cucggaaauugcgcuucuu
(SEQ ID NO: 866) ucggaaauugcgcuucuuc
(SEQ ID NO: 867) cggaaauugcgcuucuucu
(SEQ ID NO: 868) ggaaauugcgcuucuucuc
(SEQ ID NO: 869) gaaauugcgcuucuucucu
(SEQ ID NO: 870) aaauugcgcuucuucucuu
(SEQ ID NO: 871) aauugcgcuucuucucuug
(SEQ ID NO: 872) auugcgcuucuucucuugg
(SEQ ID NO: 873) uugcgcuucuucucuuggg
(SEQ ID NO: 874) ugcgcuucuucucuuggga
(SEQ ID NO: 875) gcgcuucuucucuugggau
(SEQ ID NO: 876) cgcuucuucucuugggauu
(SEQ ID NO: 877) gcuucuucucuugggauug
(SEQ ID NO: 878) cuucuucucuugggauugu
(SEQ ID NO: 879) uucuucucuugggauuguu
(SEQ ID NO: 880) ucuucucuugggauuguuu
(SEQ ID NO: 881) cuucucuugggauuguuuc
(SEQ ID NO: 882) uucucuugggauuguuuca
(SEQ ID NO: 883) ucucuugggauuguuucaa
(SEQ ID NO: 884) cucuugggauuguuucaag
(SEQ ID NO: 885) ucuugggauuguuucaaga
(SEQ ID NO: 886) cuugggauuguuucaagau
(SEQ ID NO: 887) uugggauuguuucaagauu
(SEQ ID NO: 888) ugggauuguuucaagauuu
(SEQ ID NO: 889) gggauuguuucaagauuuc
(SEQ ID NO: 890) ggauuguuucaagauuuca
(SEQ ID NO: 891) gauuguuucaagauuucag
(SEQ ID NO: 892) auuguuucaagauuucagc
(SEQ ID NO: 893) uuguuucaagauuucagcc
(SEQ ID NO: 894) uguuucaagauuucagcca
(SEQ ID NO: 895) guuucaagauuucagccac
(SEQ ID NO: 896) uuucaagauuucagccaca
(SEQ ID NO: 897) uucaagauuucagccacac
(SEQ ID NO: 898) ucaagauuucagccacacu
(SEQ ID NO: 899) caagauuucagccacacuc
(SEQ ID NO: 900) aagauuucagccacacucu
(SEQ ID NO: 901) agauuucagccacacucuc
(SEQ ID NO: 902) gauuucagccacacucucg
(SEQ ID NO: 903) auuucagccacacucucgu
(SEQ ID NO: 904) uuucagccacacucucguu
(SEQ ID NO: 905) uucagccacacucucguuc
(SEQ ID NO: 906) ucagccacacucucguuca
(SEQ ID NO: 907) cagccacacucucguucag
(SEQ ID NO: 908) agccacacucucguucagc
(SEQ ID NO: 909) gccacacucucguucagcu
(SEQ ID NO: 910) ccacacucucguucagcug
(SEQ ID NO: 911) cacacucucguucagcugg
(SEQ ID NO: 912) acacucucguucagcuggu
(SEQ ID NO: 913) cacucucguucagcugguc
(SEQ ID NO: 914) acucucguucagcugguca
(SEQ ID NO: 915) cucucguucagcuggucac
(SEQ ID NO: 916) ucucguucagcuggucacg
(SEQ ID NO: 917) cucguucagcuggucacgu
(SEQ ID NO: 918) ucguucagcuggucacgug
(SEQ ID NO: 919) cguucagcuggucacgugu
(SEQ ID NO: 920) guucagcuggucacgugug
(SEQ ID NO: 921) uucagcuggucacguguga
(SEQ ID NO: 922) ucagcuggucacgugugau
(SEQ ID NO: 923) cagcuggucacgugugauu
(SEQ ID NO: 924) agcuggucacgugugauuu
(SEQ ID NO: 925) gcuggucacgugugauuuu
(SEQ ID NO: 926) cuggucacgugugauuuug

TABLE 3
Sense siRNA sequences to hg-rps-23

(SEQ ID NO: 927) gaagcgcaauuuccgagaa
(SEQ ID NO: 928) auugcaaauuguuuugaaa
(SEQ ID NO: 929) uugcauccuuggugauuaa
(SEQ ID NO: 930) accugaagaaguugaacaa

TABLE 4

| | amiR164- | amiR164- |
|---|---|---|
| GOI | GUS | rps23-1 |
| # Root | 9 | 6 |
| events | | |
| Cysts | 38.6 | 22.7 |
| SD | 15.5 | 5.3 |

1. A double stranded RNA molecule comprising an antisense strand and a sense strand, wherein the nucleotide sequence of the antisense strand is complementary to a portion of the nucleotide sequence of a Hg-rps-23 gene of a soybean cyst nematode, the portion consisting essentially of about 18 to about 25 consecutive nucleotides of SEQ ID NO:931; wherein the double stranded RNA molecule inhibits expression of the Hg-rps-23 gene.

2. The RNA molecule of claim 1, wherein the portion of the nucleotide sequence of the Hg-rps-23 gene consists essentially of the nucleotide sequence of any of SEQ ID NOs: 1-463 (Table 1).

3. The RNA molecule of claim 1, wherein the portion of the nucleotide sequence of the Hg-rps-23 gene consists essentially of the nucleotide sequence of SEQ ID NO:64.

4. The RNA molecule of claim 1, wherein the portion of the nucleotide sequence of the Hg-rps-23 gene consists essentially of the nucleotide sequence of SEQ ID NO:258.

5. The RNA molecule of claim 1, wherein the nucleotide sequence of the antisense strand consists essentially of the nucleotide sequence of any of SEQ ID NOs:464-926 (Table 2).

6. The RNA molecule of claim 1, wherein the nucleotide sequence of the antisense strand consists essentially of the nucleotide sequence of SEQ ID NO: 863.

7. The RNA molecule of claim 1, wherein the nucleotide sequence of the antisense strand consists essentially of the nucleotide sequence of SEQ ID NO:669.

8. The RNA molecule of claim 1, wherein the nucleotide sequence of the sense strand is substantially complementary to the nucleotide sequence of the antisense strand.

9. The RNA molecule of claim 1, wherein the nucleotide sequence of the sense strand is fully complementary to the nucleotide sequence of the antisense strand.

10. The RNA molecule of claim 1, wherein the double stranded RNA molecule is a short hairpin RNA (shRNA) molecule.

11. A nucleic acid construct comprising the RNA molecule of claim 1.

12. A nucleic acid molecule encoding the RNA molecule of claim 1.

13. A nucleic acid construct comprising the nucleic acid molecule of claim 12.

14. A chimeric nucleic acid molecule comprising an antisense strand having the nucleotide sequence of any of SEQ ID NOs:464-926 operably associated with a plant microRNA precursor molecule.

15. The chimeric nucleic acid molecule of claim 14, wherein the plant microRNA precursor molecule is a soybean microRNA precursor.

16. The chimeric nucleic acid molecule of claim 15, wherein the plant microRNA precursor molecule is gma-MIR164.

17. A nucleic acid construct comprising the chimeric nucleic acid molecule of claim 14.

18. A nucleic acid molecule encoding the chimeric nucleic acid molecule of claim 14.

19. A nucleic acid construct comprising the nucleic acid molecule of claim 18.

20. An artificial plant microRNA precursor molecule comprising an antisense strand having the nucleotide sequence of any of SEQ ID Nos:464-926.

21. The artificial plant microRNA precursor molecule of claim 20, wherein the microRNA precursor molecule is a soybean microRNA precursor molecule.

22. The artificial plant microRNA precursor molecule of claim 21, wherein the microRNA precursor molecule is gma-MIR164.

23. A nucleic acid construct comprising the artificial plant microRNA precursor molecule of claim 20.

24. A nucleic acid molecule encoding the artificial plant microRNA of claim 20.

25. A nucleic acid construct comprising the nucleic acid molecule of claim 24.

26. The nucleic acid construct of claim 11 wherein the nucleic acid construct is an expression vector.

27. A composition comprising two or more of the RNA molecules of claim 1, wherein the two or more RNA molecules each comprise a different antisense strand.

28. The composition of claim 27, wherein the two or more RNA molecules are present on the same nucleic acid construct, on different nucleic acid constructs or any combination thereof.

29. The composition of claim 27, comprising an RNA molecule comprising an antisense strand consisting essentially of the nucleotide sequence of SEQ ID NO: 863 and an RNA molecule comprising an antisense strand consisting essentially of the nucleotide sequence of SEQ ID NO:669.

30. A composition comprising two or more of the nucleic acid constructs of claim 11, wherein the two or more nucleic acid constructs each comprise a different antisense strand.

31. A composition comprising two or more of the nucleic acid molecules of claim 12, wherein the two or more nucleic acid molecules each encode a different antisense strand.

32. A composition comprising two or more of the nucleic acid constructs of claim 13, wherein the two or more nucleic acid constructs each comprise a nucleic acid molecule encoding a different antisense strand.

33. A composition comprising two or more of the chimeric nucleic acid molecules of claim 14, wherein the two or more chimeric nucleic acid molecules each comprise a different antisense strand.

34. A composition comprising two or more of the artificial plant microRNA precursor molecules of claim 20, wherein the two or more artificial plant microRNA precursor molecules each comprise a different antisense strand.

35. A transformed plant cell comprising the RNA molecule of claim 1, wherein the transformed plant cell has enhanced resistance to soybean cyst nematode infection as compared to a control plant cell.

36. The plant cell of claim 35, wherein the plant cell is a legume plant cell.

37. The plant cell of claim 36, wherein the plant cell is a soybean plant cell.

38. A transgenic plant comprising the RNA molecule of claim 1, wherein the transgenic plant has enhanced resistance to soybean cyst nematode infection as compared to a control plant.

39. The transgenic plant of claim 38, wherein the transgenic plant is a legume plant.

40. The transgenic plant of claim 39, wherein the transgenic plant is a soybean plant.

41. A method of enhancing resistance of a plant cell to infection by a nematode, comprising introducing into the plant cell the RNA molecule of claim 1, thereby enhancing resistance of the plant cell to infection by the nematode.

42. A method for controlling the infection of a plant cell by a nematode, comprising contacting the nematode infecting the plant cell with the RNA molecule of claim 1, thereby controlling infection of the plant cell by the nematode.

43. A method of enhancing resistance of a plant to infection by a nematode, comprising introducing into cells of the plant the RNA molecule of claim 1, thereby enhancing resistance of the plant to infection by the nematode.

44. A method for controlling the infection of a plant by a nematode, comprising contacting the nematode infecting the plant with the RNA molecule of claim 1, thereby controlling infection of the plant by the nematode.

45. A method of reducing nematode cyst development on roots of a plant infected by a nematode, comprising introducing into cells of the plant the RNA molecule of claim 1, thereby reducing nematode cyst development on roots of the plant.

46. A method of producing a transformed plant cell having enhanced resistance to nematode infection, comprising introducing into the plant cell the RNA molecule of claim 1, thereby producing a transformed plant cell having enhanced resistance to nematode infection relative to a control plant cell.

47. A transformed plant cell produced by the method of claim 46.

48. A method of producing a transgenic plant having enhanced resistance to nematode infection, comprising transforming cells of the plant with the RNA molecule of claim 1, thereby producing a transgenic plant having enhanced resistance to nematode infection relative to a control plant.

49. A transgenic plant produced by the method of claim 48,

50. A method of making a transgenic plant having enhanced resistance to nematode infection, comprising:
a) transforming a plant cell with the RNA molecule of claim 1 to produce a transformed plant cell; and
b) growing the transformed plant cell into a transgenic plant, whereby the transgenic plant has enhanced resistant to nematode infection relative to a control plant.

51. A transgenic plant produced by the method of claim 50.

52. A progeny plant of the transgenic plant of claim 49, wherein the progeny plant is a transgenic plant.

53. A seed of the transgenic plant of claim 49, wherein the seed is a transgenic seed,

54. The method of claim 41, wherein the plant cell is a legume plant cell.

55. The method of claim 54, wherein the plant cell is a soybean plant cell.

56. The method of claim 43, wherein the plant is a legume plant.

57. The method of claim 56, wherein the plant is a soybean plant.

58. The method of claim 41, wherein the nematode is a soybean cyst nematode.

59. A crop comprising a plurality of the transgenic plant of claim 38, planted together in an agricultural field.

60. A method of improving crop yield, comprising:
a) introducing the RNA molecule of claim 1 into cells of a plant; and
b) cultivating a plurality of the plant of (a) as a crop, resulting in a plurality of plants having enhanced resistance to nematode infection, thereby improving crop yield.

61. The crop of claim 59, wherein the plant is a legume plant.

62. The crop of claim 59, wherein the plant is a soybean plant.


SEQUENCE LISTING (937 sequences)

SEQ ID NO: 1 - DNA, 19 bp, Artificial
  siRNA target sequence
caaaatcacacgtgaccag

SEQ ID NO: 2 - DNA, 19 bp, Artificial
  siRNA target sequence
aaaatcacacgtgaccagc

SEQ ID NO: 3 - DNA, 19 bp, Artificial
  siRNA target sequence
aaatcacacgtgaccagct

SEQ ID NO: 4 - DNA, 19 bp, Artificial
  siRNA target sequence
aatcacacgtgaccagctg

SEQ ID NO: 5 - DNA, 19 bp, Artificial
  siRNA target sequence
atcacacgtgaccagctga

SEQ ID NO: 6 - DNA, 19 bp, Artificial
  siRNA target sequence
tcacacgtgaccagctgaa

SEQ ID NO: 7 - DNA, 19 bp, Artificial
  siRNA target sequence
cacacgtgaccagctgaac

SEQ ID NO: 8 - DNA, 19 bp, Artificial
  siRNA target sequence
acacgtgaccagctgaacg

SEQ ID NO: 9 - DNA, 19 bp, Artificial
  siRNA target sequence
cacgtgaccagctgaacga

SEQ ID NO: 10 - DNA, 19 bp, Artificial
  siRNA target sequence
acgtgaccagctgaacgag

SEQ ID NO: 11 - DNA, 19 bp, Artificial
  siRNA target sequence
cgtgaccagctgaacgaga

SEQ ID NO: 12 - DNA, 19 bp, Artificial
  siRNA target sequence
gtgaccagctgaacgagag

SEQ ID NO: 13 - DNA, 19 bp, Artificial
  siRNA target sequence
tgaccagctgaacgagagt

SEQ ID NO: 14 - DNA, 19 bp, Artificial
  siRNA target sequence
gaccagctgaacgagagtg

SEQ ID NO: 15 - DNA, 19 bp, Artificial
  siRNA target sequence
accagctgaacgagagtgt

SEQ ID NO: 16 - DNA, 19 bp, Artificial
  siRNA target sequence
ccagctgaacgagagtgtg

SEQ ID NO: 17 - DNA, 19 bp, Artificial
  siRNA target sequence
cagctgaacgagagtgtgg

SEQ ID NO: 18 - DNA, 19 bp, Artificial
  siRNA target sequence
agctgaacgagagtgtggc

SEQ ID NO: 19 - DNA, 19 bp, Artificial
  siRNA target sequence
gctgaacgagagtgtggct

SEQ ID NO: 20 - DNA, 19 bp, Artificial
  siRNA target sequence
ctgaacgagagtgtggctg

SEQ ID NO: 21 - DNA, 19 bp, Artificial
  siRNA target sequence
tgaacgagagtgtggctga

SEQ ID NO: 22 - DNA, 19 bp, Artificial
  siRNA target sequence
gaacgagagtgtggctgaa

SEQ ID NO: 23 - DNA, 19 bp, Artificial
  siRNA target sequence
aacgagagtgtggctgaaa

SEQ ID NO: 24 - DNA, 19 bp, Artificial
  siRNA target sequence
acgagagtgtggctgaaat

SEQ ID NO: 25 - DNA, 19 bp, Artificial
  siRNA target sequence
cgagagtgtggctgaaatc

SEQ ID NO: 26 - DNA, 19 bp, Artificial
  siRNA target sequence
gagagtgtggctgaaatct

SEQ ID NO: 27 - DNA, 19 bp, Artificial
  siRNA target sequence
agagtgtggctgaaatctt

SEQ ID NO: 28 - DNA, 19 bp, Artificial
  siRNA target sequence
gagtgtggctgaaatcttg

SEQ ID NO: 29 - DNA, 19 bp, Artificial
  siRNA target sequence
agtgtggctgaaatcttga

SEQ ID NO: 30 - DNA, 19 bp, Artificial
  siRNA target sequence
gtgtggctgaaatcttgaa

SEQ ID NO: 31 - DNA, 19 bp, Artificial
  siRNA target sequence
tgtggctgaaatcttgaaa

SEQ ID NO: 32 - DNA, 19 bp, Artificial
  siRNA target sequence
gtggctgaaatcttgaaac

SEQ ID NO: 33 - DNA, 19 bp, Artificial
  siRNA target sequence
tggctgaaatcttgaaaca

SEQ ID NO: 34 - DNA, 19 bp, Artificial
  siRNA target sequence
ggctgaaatcttgaaacaa

SEQ ID NO: 35 - DNA, 19 bp, Artificial
  siRNA target sequence
gctgaaatcttgaaacaat

SEQ ID NO: 36 - DNA, 19 bp, Artificial
  siRNA target sequence
ctgaaatcttgaaacaatc

SEQ ID NO: 37 - DNA, 19 bp, Artificial
  siRNA target sequence
tgaaatcttgaaacaatcc

SEQ ID NO: 38 - DNA, 19 bp, Artificial
  siRNA target sequence
gaaatcttgaaacaatccc

SEQ ID NO: 39 - DNA, 19 bp, Artificial
  siRNA target sequence
aaatcttgaaacaatccca

SEQ ID NO: 40 - DNA, 19 bp, Artificial
  siRNA target sequence
aatcttgaaacaatcccaa

SEQ ID NO: 41 - DNA, 19 bp, Artificial
  siRNA target sequence
atcttgaaacaatcccaag

SEQ ID NO: 42 - DNA, 19 bp, Artificial
  siRNA target sequence
tcttgaaacaatcccaaga

SEQ ID NO: 43 - DNA, 19 bp, Artificial
  siRNA target sequence
cttgaaacaatcccaagag

SEQ ID NO: 44 - DNA, 19 bp, Artificial
  siRNA target sequence
ttgaaacaatcccaagaga

SEQ ID NO: 45 - DNA, 19 bp, Artificial
  siRNA target sequence
tgaaacaatcccaagagaa

SEQ ID NO: 46 - DNA, 19 bp, Artificial
  siRNA target sequence
gaaacaatcccaagagaag

SEQ ID NO: 47 - DNA, 19 bp, Artificial
  siRNA target sequence
aaacaatcccaagagaaga

SEQ ID NO: 48 - DNA, 19 bp, Artificial
  siRNA target sequence
aacaatcccaagagaagaa

SEQ ID NO: 49 - DNA, 19 bp, Artificial
  siRNA target sequence
acaatcccaagagaagaag

SEQ ID NO: 50 - DNA, 19 bp, Artificial
  siRNA target sequence
caatcccaagagaagaagc

SEQ ID NO: 51 - DNA, 19 bp, Artificial
  siRNA target sequence
aatcccaagagaagaagcg

SEQ ID NO: 52 - DNA, 19 bp, Artificial
  siRNA target sequence
atcccaagagaagaagcgc

SEQ ID NO: 53 - DNA, 19 bp, Artificial
  siRNA target sequence
tcccaagagaagaagcgca

SEQ ID NO: 54 - DNA, 19 bp, Artificial
  siRNA target sequence
cccaagagaagaagcgcaa

SEQ ID NO: 55 - DNA, 19 bp, Artificial
  siRNA target sequence
ccaagagaagaagcgcaat

SEQ ID NO: 56 - DNA, 19 bp, Artificial
  siRNA target sequence
caagagaagaagcgcaatt

SEQ ID NO: 57 - DNA, 19 bp, Artificial
  siRNA target sequence
aagagaagaagcgcaattt

SEQ ID NO: 58 - DNA, 19 bp, Artificial
  siRNA target sequence
agagaagaagcgcaatttc

SEQ ID NO: 59 - DNA, 19 bp, Artificial
  siRNA target sequence
gagaagaagcgcaatttcc

SEQ ID NO: 60 - DNA, 19 bp, Artificial
  siRNA target sequence
agaagaagcgcaatttccg

SEQ ID NO: 61 - DNA, 19 bp, Artificial
  siRNA target sequence
gaagaagcgcaatttccga

SEQ ID NO: 62 - DNA, 19 bp, Artificial
  siRNA target sequence
aagaagcgcaatttccgag

SEQ ID NO: 63 - DNA, 19 bp, Artificial
  siRNA target sequence
agaagcgcaatttccgaga

SEQ ID NO: 64 - DNA, 19 bp, Artificial
  siRNA target sequence
gaagcgcaatttccgagaa

SEQ ID NO: 65 - DNA, 19 bp, Artificial
  siRNA target sequence
aagcgcaatttccgagaaa

SEQ ID NO: 66 - DNA, 19 bp, Artificial
  siRNA target sequence
agcgcaatttccgagaaac

SEQ ID NO: 67 - DNA, 19 bp, Artificial
  siRNA target sequence
gcgcaatttccgagaaacg

SEQ ID NO: 68 - DNA, 19 bp, Artificial
  siRNA target sequence
cgcaatttccgagaaacga

SEQ ID NO: 69 - DNA, 19 bp, Artificial
  siRNA target sequence
gcaatttccgagaaacgat

SEQ ID NO: 70 - DNA, 19 bp, Artificial
  siRNA target sequence
caatttccgagaaacgatt

SEQ ID NO: 71 - DNA, 19 bp, Artificial
  siRNA target sequence
aatttccgagaaacgattg

SEQ ID NO: 72 - DNA, 19 bp, Artificial
  siRNA target sequence
atttccgagaaacgattga

SEQ ID NO: 73 - DNA, 19 bp, Artificial
  siRNA target sequence
tttccgagaaacgattgaa

SEQ ID NO: 74 - DNA, 19 bp, Artificial
  siRNA target sequence
ttccgagaaacgattgaat

SEQ ID NO: 75 - DNA, 19 bp, Artificial
  siRNA target sequence
tccgagaaacgattgaatt

SEQ ID NO: 76 - DNA, 19 bp, Artificial
  siRNA target sequence
ccgagaaacgattgaattg

SEQ ID NO: 77 - DNA, 19 bp, Artificial
  siRNA target sequence
cgagaaacgattgaattgc

SEQ ID NO: 78 - DNA, 19 bp, Artificial
  siRNA target sequence
gagaaacgattgaattgca

SEQ ID NO: 79 - DNA, 19 bp, Artificial
  siRNA target sequence
agaaacgattgaattgcaa

SEQ ID NO: 80 - DNA, 19 bp, Artificial
  siRNA target sequence
gaaacgattgaattgcaaa

SEQ ID NO: 81 - DNA, 19 bp, Artificial
  siRNA target sequence
aaacgattgaattgcaaat

SEQ ID NO: 82 - DNA, 19 bp, Artificial
  siRNA target sequence
aacgattgaattgcaaatt

SEQ ID NO: 83 - DNA, 19 bp, Artificial
  siRNA target sequence
acgattgaattgcaaattg

SEQ ID NO: 84 - DNA, 19 bp, Artificial
  siRNA target sequence
cgattgaattgcaaattgc

SEQ ID NO: 85 - DNA, 19 bp, Artificial
  siRNA target sequence
gattgaattgcaaattgct

SEQ ID NO: 86 - DNA, 19 bp, Artificial
  siRNA target sequence
attgaattgcaaattgctc

SEQ ID NO: 87 - DNA, 19 bp, Artificial
  siRNA target sequence
ttgaattgcaaattgctct

SEQ ID NO: 88 - DNA, 19 bp, Artificial
  siRNA target sequence
tgaattgcaaattgctctg

SEQ ID NO: 89 - DNA, 19 bp, Artificial
  siRNA target sequence
gaattgcaaattgctctga

SEQ ID NO: 90 - DNA, 19 bp, Artificial
  siRNA target sequence
aattgcaaattgctctgaa

SEQ ID NO: 91 - DNA, 19 bp, Artificial
  siRNA target sequence
attgcaaattgctctgaaa

SEQ ID NO: 92 - DNA, 19 bp, Artificial
  siRNA target sequence
ttgcaaattgctctgaaaa

SEQ ID NO: 93 - DNA, 19 bp, Artificial
  siRNA target sequence
tgcaaattgctctgaaaaa

SEQ ID NO: 94 - DNA, 19 bp, Artificial
  siRNA target sequence
gcaaattgctctgaaaaac

SEQ ID NO: 95 - DNA, 19 bp, Artificial
  siRNA target sequence
caaattgctctgaaaaact

SEQ ID NO: 96 - DNA, 19 bp, Artificial
  siRNA target sequence
aaattgctctgaaaaacta

SEQ ID NO: 97 - DNA, 19 bp, Artificial
  siRNA target sequence
aattgctctgaaaaactac

SEQ ID NO: 98 - DNA, 19 bp, Artificial
  siRNA target sequence
attgctctgaaaaactacg

SEQ ID NO: 99 - DNA, 19 bp, Artificial
  siRNA target sequence
ttgctctgaaaaactacga

SEQ ID NO: 100 - DNA, 19 bp, Artificial
  siRNA target sequence
tgctctgaaaaactacgac

SEQ ID NO: 101 - DNA, 19 bp, Artificial
  siRNA target sequence
gctctgaaaaactacgacc

SEQ ID NO: 102 - DNA, 19 bp, Artificial
  siRNA target sequence
ctctgaaaaactacgaccc

SEQ ID NO: 103 - DNA, 19 bp, Artificial
  siRNA target sequence
tctgaaaaactacgaccca

SEQ ID NO: 104 - DNA, 19 bp, Artificial
  siRNA target sequence
ctgaaaaactacgacccac

SEQ ID NO: 105 - DNA, 19 bp, Artificial
  siRNA target sequence
tgaaaaactacgacccaca

SEQ ID NO: 106 - DNA, 19 bp, Artificial
  siRNA target sequence
gaaaaactacgacccacag

SEQ ID NO: 107 - DNA, 19 bp, Artificial
  siRNA target sequence
aaaaactacgacccacaga

SEQ ID NO: 108 - DNA, 19 bp, Artificial
  siRNA target sequence
aaaactacgacccacagaa

SEQ ID NO: 109 - DNA, 19 bp, Artificial
  siRNA target sequence
aaactacgacccacagaag

SEQ ID NO: 110 - DNA, 19 bp, Artificial
  siRNA target sequence
aactacgacccacagaagg

SEQ ID NO: 111 - DNA, 19 bp, Artificial
  siRNA target sequence
actacgacccacagaagga

SEQ ID NO: 112 - DNA, 19 bp, Artificial
  siRNA target sequence
ctacgacccacagaaggac

SEQ ID NO: 113 - DNA, 19 bp, Artificial
  siRNA target sequence
tacgacccacagaaggaca

SEQ ID NO: 114 - DNA, 19 bp, Artificial
  siRNA target sequence
acgacccacagaaggacaa

SEQ ID NO: 115 - DNA, 19 bp, Artificial
  siRNA target sequence
cgacccacagaaggacaag

SEQ ID NO: 116 - DNA, 19 bp, Artificial
  siRNA target sequence
gacccacagaaggacaagc

SEQ ID NO: 117 - DNA, 19 bp, Artificial
  siRNA target sequence
acccacagaaggacaagcg

SEQ ID NO: 118 - DNA, 19 bp, Artificial
  siRNA target sequence
cccacagaaggacaagcgt

SEQ ID NO: 119 - DNA, 19 bp, Artificial
  siRNA target sequence
ccacagaaggacaagcgtt

SEQ ID NO: 120 - DNA, 19 bp, Artificial
  siRNA target sequence
cacagaaggacaagcgttt

SEQ ID NO: 121 - DNA, 19 bp, Artificial
  siRNA target sequence
acagaaggacaagcgtttc

SEQ ID NO: 122 - DNA, 19 bp, Artificial
  siRNA target sequence
cagaaggacaagcgtttca

SEQ ID NO: 123 - DNA, 19 bp, Artificial
  siRNA target sequence
agaaggacaagcgtttcag

SEQ ID NO: 124 - DNA, 19 bp, Artificial
  siRNA target sequence
gaaggacaagcgtttcagt

SEQ ID NO: 125 - DNA, 19 bp, Artificial
  siRNA target sequence
aaggacaagcgtttcagtg

SEQ ID NO: 126 - DNA, 19 bp, Artificial
  siRNA target sequence
aggacaagcgtttcagtgg

SEQ ID NO: 127 - DNA, 19 bp, Artificial
  siRNA target sequence
ggacaagcgtttcagtgga

SEQ ID NO: 128 - DNA, 19 bp, Artificial
  siRNA target sequence
gacaagcgtttcagtggaa

SEQ ID NO: 129 - DNA, 19 bp, Artificial
  siRNA target sequence
acaagcgtttcagtggaac

SEQ ID NO: 130 - DNA, 19 bp, Artificial
  siRNA target sequence
caagcgtttcagtggaact

SEQ ID NO: 131 - DNA, 19 bp, Artificial
  siRNA target sequence
aagcgtttcagtggaactg

SEQ ID NO: 132 - DNA, 19 bp, Artificial
  siRNA target sequence
agcgtttcagtggaactgt

SEQ ID NO: 133 - DNA, 19 bp, Artificial
  siRNA target sequence
gcgtttcagtggaactgtt

SEQ ID NO: 134 - DNA, 19 bp, Artificial
  siRNA target sequence
cgtttcagtggaactgtta

SEQ ID NO: 135 - DNA, 19 bp, Artificial
  siRNA target sequence
gtttcagtggaactgttag

SEQ ID NO: 136 - DNA, 19 bp, Artificial
  siRNA target sequence
tttcagtggaactgttaga

SEQ ID NO: 137 - DNA, 19 bp, Artificial
  siRNA target sequence
ttcagtggaactgttagac

SEQ ID NO: 138 - DNA, 19 bp, Artificial
  siRNA target sequence
tcagtggaactgttagact

SEQ ID NO: 139 - DNA, 19 bp, Artificial
  siRNA target sequence
cagtggaactgttagactg

SEQ ID NO: 140 - DNA, 19 bp, Artificial
  siRNA target sequence
agtggaactgttagactga

SEQ ID NO: 141 - DNA, 19 bp, Artificial
  siRNA target sequence
gtggaactgttagactgaa

SEQ ID NO: 142 - DNA, 19 bp, Artificial
  siRNA target sequence
tggaactgttagactgaag

SEQ ID NO: 143 - DNA, 19 bp, Artificial
  siRNA target sequence
ggaactgttagactgaagc

SEQ ID NO: 144 - DNA, 19 bp, Artificial
  siRNA target sequence
gaactgttagactgaagca

SEQ ID NO: 145 - DNA, 19 bp, Artificial
  siRNA target sequence
aactgttagactgaagcac

SEQ ID NO: 146 - DNA, 19 bp, Artificial
  siRNA target sequence
actgttagactgaagcaca

SEQ ID NO: 147 - DNA, 19 bp, Artificial
  siRNA target sequence
ctgttagactgaagcacat

SEQ ID NO: 148 - DNA, 19 bp, Artificial
  siRNA target sequence
tgttagactgaagcacatc

SEQ ID NO: 149 - DNA, 19 bp, Artificial
  siRNA target sequence
gttagactgaagcacatcc

SEQ ID NO: 150 - DNA, 19 bp, Artificial
  siRNA target sequence
ttagactgaagcacatccc

SEQ ID NO: 151 - DNA, 19 bp, Artificial
  siRNA target sequence
tagactgaagcacatccct

SEQ ID NO: 152 - DNA, 19 bp, Artificial
  siRNA target sequence
agactgaagcacatccctc

SEQ ID NO: 153 - DNA, 19 bp, Artificial
  siRNA target sequence
gactgaagcacatccctcg

SEQ ID NO: 154 - DNA, 19 bp, Artificial
  siRNA target sequence
actgaagcacatccctcgt

SEQ ID NO: 155 - DNA, 19 bp, Artificial
  siRNA target sequence
ctgaagcacatccctcgtc

SEQ ID NO: 156 - DNA, 19 bp, Artificial
  siRNA target sequence
tgaagcacatccctcgtcc

SEQ ID NO: 157 - DNA, 19 bp, Artificial
  siRNA target sequence
gaagcacatccctcgtccg

SEQ ID NO: 158 - DNA, 19 bp, Artificial
  siRNA target sequence
aagcacatccctcgtccga

SEQ ID NO: 159 - DNA, 19 bp, Artificial
  siRNA target sequence
agcacatccctcgtccgaa

SEQ ID NO: 160 - DNA, 19 bp, Artificial
  siRNA target sequence
gcacatccctcgtccgaaa

SEQ ID NO: 161 - DNA, 19 bp, Artificial
  siRNA target sequence
cacatccctcgtccgaaaa

SEQ ID NO: 162 - DNA, 19 bp, Artificial
  siRNA target sequence
acatccctcgtccgaaaac

SEQ ID NO: 163 - DNA, 19 bp, Artificial
  siRNA target sequence
catccctcgtccgaaaacg

SEQ ID NO: 164 - DNA, 19 bp, Artificial
  siRNA target sequence
atccctcgtccgaaaacga

SEQ ID NO: 165 - DNA, 19 bp, Artificial
  siRNA target sequence
tccctcgtccgaaaacgaa

SEQ ID NO: 166 - DNA, 19 bp, Artificial
  siRNA target sequence
ccctcgtccgaaaacgaag

SEQ ID NO: 167 - DNA, 19 bp, Artificial
  siRNA target sequence
cctcgtccgaaaacgaagg

SEQ ID NO: 168 - DNA, 19 bp, Artificial
  siRNA target sequence
ctcgtccgaaaacgaaggt

SEQ ID NO: 169 - DNA, 19 bp, Artificial
  siRNA target sequence
tcgtccgaaaacgaaggtt

SEQ ID NO: 170 - DNA, 19 bp, Artificial
  siRNA target sequence
cgtccgaaaacgaaggttt

SEQ ID NO: 171 - DNA, 19 bp, Artificial
  siRNA target sequence
gtccgaaaacgaaggtttg

SEQ ID NO: 172 - DNA, 19 bp, Artificial
  siRNA target sequence
tccgaaaacgaaggtttgc

SEQ ID NO: 173 - DNA, 19 bp, Artificial
  siRNA target sequence
ccgaaaacgaaggtttgca

SEQ ID NO: 174 - DNA, 19 bp, Artificial
  siRNA target sequence
cgaaaacgaaggtttgcat

SEQ ID NO: 175 - DNA, 19 bp, Artificial
  siRNA target sequence
gaaaacgaaggtttgcatc

SEQ ID NO: 176 - DNA, 19 bp, Artificial
  siRNA target sequence
aaaacgaaggtttgcatcc

SEQ ID NO: 177 - DNA, 19 bp, Artificial
  siRNA target sequence
aaacgaaggtttgcatcct

SEQ ID NO: 178 - DNA, 19 bp, Artificial
  siRNA target sequence
aacgaaggtttgcatcctt

SEQ ID NO: 179 - DNA, 19 bp, Artificial
  siRNA target sequence
acgaaggtttgcatccttg

SEQ ID NO: 180 - DNA, 19 bp, Artificial
  siRNA target sequence
cgaaggtttgcatccttgg

SEQ ID NO: 181 - DNA, 19 bp, Artificial
  siRNA target sequence
gaaggtttgcatccttggc

SEQ ID NO: 182 - DNA, 19 bp, Artificial
  siRNA target sequence
aaggtttgcatccttggcg

SEQ ID NO: 183 - DNA, 19 bp, Artificial
  siRNA target sequence
aggtttgcatccttggcga

SEQ ID NO: 184 - DNA, 19 bp, Artificial
  siRNA target sequence
ggtttgcatccttggcgac

SEQ ID NO: 185 - DNA, 19 bp, Artificial
  siRNA target sequence
gtttgcatccttggcgacc

SEQ ID NO: 186 - DNA, 19 bp, Artificial
  siRNA target sequence
tttgcatccttggcgacca

SEQ ID NO: 187 - DNA, 19 bp, Artificial
  siRNA target sequence
ttgcatccttggcgaccaa

SEQ ID NO: 188 - DNA, 19 bp, Artificial
  siRNA target sequence
tgcatccttggcgaccaaa

SEQ ID NO: 189 - DNA, 19 bp, Artificial
  siRNA target sequence
gcatccttggcgaccaaaa

SEQ ID NO: 190 - DNA, 19 bp, Artificial
  siRNA target sequence
catccttggcgaccaaaaa

SEQ ID NO: 191 - DNA, 19 bp, Artificial
  siRNA target sequence
atccttggcgaccaaaaac

SEQ ID NO: 192 - DNA, 19 bp, Artificial
  siRNA target sequence
tccttggcgaccaaaaaca

SEQ ID NO: 193 - DNA, 19 bp, Artificial
  siRNA target sequence
ccttggcgaccaaaaacat

SEQ ID NO: 194 - DNA, 19 bp, Artificial
  siRNA target sequence
cttggcgaccaaaaacatt

SEQ ID NO: 195 - DNA, 19 bp, Artificial
  siRNA target sequence
ttggcgaccaaaaacattg

SEQ ID NO: 196 - DNA, 19 bp, Artificial
  siRNA target sequence
tggcgaccaaaaacattgt

SEQ ID NO: 197 - DNA, 19 bp, Artificial
  siRNA target sequence
ggcgaccaaaaacattgtg

SEQ ID NO: 198 - DNA, 19 bp, Artificial
  siRNA target sequence
gcgaccaaaaacattgtga

SEQ ID NO: 199 - DNA, 19 bp, Artificial
  siRNA target sequence
cgaccaaaaacattgtgac

SEQ ID NO: 200 - DNA, 19 bp, Artificial
  siRNA target sequence
gaccaaaaacattgtgacg

SEQ ID NO: 201 - DNA, 19 bp, Artificial
  siRNA target sequence
accaaaaacattgtgacga

SEQ ID NO: 202 - DNA, 19 bp, Artificial
  siRNA target sequence
ccaaaaacattgtgacgag

SEQ ID NO: 203 - DNA, 19 bp, Artificial
  siRNA target sequence
caaaaacattgtgacgagg

SEQ ID NO: 204 - DNA, 19 bp, Artificial
  siRNA target sequence
aaaaacattgtgacgaggc

SEQ ID NO: 205 - DNA, 19 bp, Artificial
  siRNA target sequence
aaaacattgtgacgaggcc

SEQ ID NO: 206 - DNA, 19 bp, Artificial
  siRNA target sequence
aaacattgtgacgaggcca

SEQ ID NO: 207 - DNA, 19 bp, Artificial
  siRNA target sequence
aacattgtgacgaggccaa

SEQ ID NO: 208 - DNA, 19 bp, Artificial
  siRNA target sequence
acattgtgacgaggccaat

SEQ ID NO: 209 - DNA, 19 bp, Artificial
  siRNA target sequence
cattgtgacgaggccaatg

SEQ ID NO: 210 - DNA, 19 bp, Artificial
  siRNA target sequence
attgtgacgaggccaatgc

SEQ ID NO: 211 - DNA, 19 bp, Artificial
  siRNA target sequence
ttgtgacgaggccaatgcc

SEQ ID NO: 212 - DNA, 19 bp, Artificial
  siRNA target sequence
tgtgacgaggccaatgcca

SEQ ID NO: 213 - DNA, 19 bp, Artificial
  siRNA target sequence
gtgacgaggccaatgccaa

SEQ ID NO: 214 - DNA, 19 bp, Artificial
  siRNA target sequence
tgacgaggccaatgccaac

SEQ ID NO: 215 - DNA, 19 bp, Artificial
  siRNA target sequence
gacgaggccaatgccaacg

SEQ ID NO: 216 - DNA, 19 bp, Artificial
  siRNA target sequence
acgaggccaatgccaacgg

SEQ ID NO: 217 - DNA, 19 bp, Artificial
  siRNA target sequence
cgaggccaatgccaacgga

SEQ ID NO: 218 - DNA, 19 bp, Artificial
  siRNA target sequence
gaggccaatgccaacggaa

SEQ ID NO: 219 - DNA, 19 bp, Artificial
  siRNA target sequence
aggccaatgccaacggaat

SEQ ID NO: 220 - DNA, 19 bp, Artificial
  siRNA target sequence
ggccaatgccaacggaatt

SEQ ID NO: 221 - DNA, 19 bp, Artificial
  siRNA target sequence
gccaatgccaacggaattc

SEQ ID NO: 222 - DNA, 19 bp, Artificial
  siRNA target sequence
ccaatgccaacggaattcc

SEQ ID NO: 223 - DNA, 19 bp, Artificial
  siRNA target sequence
caatgccaacggaattcca

SEQ ID NO: 224 - DNA, 19 bp, Artificial
  siRNA target sequence
aatgccaacggaattccat

SEQ ID NO: 225 - DNA, 19 bp, Artificial
  siRNA target sequence
atgccaacggaattccatg

SEQ ID NO: 226 - DNA, 19 bp, Artificial
  siRNA target sequence
tgccaacggaattccatgc

SEQ ID NO: 227 - DNA, 19 bp, Artificial
  siRNA target sequence
gccaacggaattccatgca

SEQ ID NO: 228 - DNA, 19 bp, Artificial
  siRNA target sequence
ccaacggaattccatgcat

SEQ ID NO: 229 - DNA, 19 bp, Artificial
  siRNA target sequence
caacggaattccatgcatg

SEQ ID NO: 230 - DNA, 19 bp, Artificial
  siRNA target sequence
aacggaattccatgcatga

SEQ ID NO: 231 - DNA, 19 bp, Artificial
  siRNA target sequence
acggaattccatgcatgac

SEQ ID NO: 232 - DNA, 19 bp, Artificial
  siRNA target sequence
cggaattccatgcatgaca

SEQ ID NO: 233 - DNA, 19 bp, Artificial
  siRNA target sequence
ggaattccatgcatgacag

SEQ ID NO: 234 - DNA, 19 bp, Artificial
  siRNA target sequence
gaattccatgcatgacagc

SEQ ID NO: 235 - DNA, 19 bp, Artificial
  siRNA target sequence
aattccatgcatgacagcg

SEQ ID NO: 236 - DNA, 19 bp, Artificial
  siRNA target sequence
attccatgcatgacagcgg

SEQ ID NO: 237 - DNA, 19 bp, Artificial
  siRNA target sequence
ttccatgcatgacagcgga

SEQ ID NO: 238 - DNA, 19 bp, Artificial
  siRNA target sequence
tccatgcatgacagcggac

SEQ ID NO: 239 - DNA, 19 bp, Artificial
  siRNA target sequence
ccatgcatgacagcggacg

SEQ ID NO: 240 - DNA, 19 bp, Artificial
  siRNA target sequence
catgcatgacagcggacga

SEQ ID NO: 241 - DNA, 19 bp, Artificial
  siRNA target sequence
atgcatgacagcggacgac

SEQ ID NO: 242 - DNA, 19 bp, Artificial
  siRNA target sequence
atgcatgacagcggacgac

SEQ ID NO: 243 - DNA, 19 bp, Artificial
  siRNA target sequence
gcatgacagcggacgacct

SEQ ID NO: 244 - DNA, 19 bp, Artificial
  siRNA target sequence
catgacagcggacgacctg

SEQ ID NO: 245 - DNA, 19 bp, Artificial
  siRNA target sequence
atgacagcggacgacctga

SEQ ID NO: 246 - DNA, 19 bp, Artificial
  siRNA target sequence
tgacagcggacgacctgaa

SEQ ID NO: 247 - DNA, 19 bp, Artificial
  siRNA target sequence
gacagcggacgacctgaag

SEQ ID NO: 248 - DNA, 19 bp, Artificial
  siRNA target sequence
acagcggacgacctgaaga

SEQ ID NO: 249 - DNA, 19 bp, Artificial
  siRNA target sequence
cagcggacgacctgaagaa

SEQ ID NO: 250 - DNA, 19 bp, Artificial
  siRNA target sequence
agcggacgacctgaagaag

SEQ ID NO: 251 - DNA, 19 bp, Artificial
  siRNA target sequence
gcggacgacctgaagaagt

SEQ ID NO: 252 - DNA, 19 bp, Artificial
  siRNA target sequence
cggacgacctgaagaagtt

SEQ ID NO: 253 - DNA, 19 bp, Artificial
  siRNA target sequence
ggacgacctgaagaagttg

SEQ ID NO: 254 - DNA, 19 bp, Artificial
  siRNA target sequence
gacgacctgaagaagttga

SEQ ID NO: 255 - DNA, 19 bp, Artificial
  siRNA target sequence
acgacctgaagaagttgaa

SEQ ID NO: 256 - DNA, 19 bp, Artificial
  siRNA target sequence
cgacctgaagaagttgaac

SEQ ID NO: 257 - DNA, 19 bp, Artificial
  siRNA target sequence
gacctgaagaagttgaaca

SEQ ID NO: 258 - DNA, 19 bp, Artificial
  siRNA target sequence
acctgaagaagttgaacaa

SEQ ID NO: 259 - DNA, 19 bp, Artificial
  siRNA target sequence
cctgaagaagttgaacaag

SEQ ID NO: 260 - DNA, 19 bp, Artificial
  siRNA target sequence
ctgaagaagttgaacaagg

SEQ ID NO: 261 - DNA, 19 bp, Artificial
  siRNA target sequence
tgaagaagttgaacaagga

SEQ ID NO: 262 - DNA, 19 bp, Artificial
  siRNA target sequence
gaagaagttgaacaaggac

SEQ ID NO: 263 - DNA, 19 bp, Artificial
  siRNA target sequence
aagaagttgaacaaggaca

SEQ ID NO: 264 - DNA, 19 bp, Artificial
  siRNA target sequence
agaagttgaacaaggacaa

SEQ ID NO: 265 - DNA, 19 bp, Artificial
  siRNA target sequence
gaagttgaacaaggacaag

SEQ ID NO: 266 - DNA, 19 bp, Artificial
  siRNA target sequence
aagttgaacaaggacaaga

SEQ ID NO: 267 - DNA, 19 bp, Artificial
  siRNA target sequence
agttgaacaaggacaagaa

SEQ ID NO: 268 - DNA, 19 bp, Artificial
  siRNA target sequence
gttgaacaaggacaagaag

SEQ ID NO: 269 - DNA, 19 bp, Artificial
  siRNA target sequence
ttgaacaaggacaagaagc

SEQ ID NO: 270 - DNA, 19 bp, Artificial
  siRNA target sequence
tgaacaaggacaagaagct

SEQ ID NO: 271 - DNA, 19 bp, Artificial
  siRNA target sequence
gaacaaggacaagaagctg

SEQ ID NO: 272 - DNA, 19 bp, Artificial
  siRNA target sequence
aacaaggacaagaagctga

SEQ ID NO: 273 - DNA, 19 bp, Artificial
  siRNA target sequence
acaaggacaagaagctgat

SEQ ID NO: 274 - DNA, 19 bp, Artificial
  siRNA target sequence
caaggacaagaagctgatc

SEQ ID NO: 275 - DNA, 19 bp, Artificial
  siRNA target sequence
aaggacaagaagctgatct

SEQ ID NO: 276 - DNA, 19 bp, Artificial
  siRNA target sequence
aggacaagaagctgatcta

SEQ ID NO: 277 - DNA, 19 bp, Artificial
  siRNA target sequence
ggacaagaagctgatctaa

SEQ ID NO: 278 - DNA, 19 bp, Artificial
  siRNA target sequence
gacaagaagctgatctaaa

SEQ ID NO: 279 - DNA, 19 bp, Artificial
  siRNA target sequence
acaagaagctgatctaaaa

SEQ ID NO: 280 - DNA, 19 bp, Artificial
  siRNA target sequence
caagaagctgatctaaaag

SEQ ID NO: 281 - DNA, 19 bp, Artificial
  siRNA target sequence
aagaagctgatctaaaagc

SEQ ID NO: 282 - DNA, 19 bp, Artificial
  siRNA target sequence
agaagctgatctaaaagct

SEQ ID NO: 283 - DNA, 19 bp, Artificial
  siRNA target sequence
gaagctgatctaaaagctc

SEQ ID NO: 284 - DNA, 19 bp, Artificial
  siRNA target sequence
aagctgatctaaaagctca

SEQ ID NO: 285 - DNA, 19 bp, Artificial
  siRNA target sequence
agctgatctaaaagctcag

SEQ ID NO: 286 - DNA, 19 bp, Artificial
  siRNA target sequence
gctgatctaaaagctcagc

SEQ ID NO: 287 - DNA, 19 bp, Artificial
  siRNA target sequence
ctgatctaaaagctcagca

SEQ ID NO: 288 - DNA, 19 bp, Artificial
  siRNA target sequence
tgatctaaaagctcagcaa

SEQ ID NO: 289 - DNA, 19 bp, Artificial
  siRNA target sequence
gatctaaaagctcagcaaa

SEQ ID NO: 290 - DNA, 19 bp, Artificial
  siRNA target sequence
atctaaaagctcagcaaaa

SEQ ID NO: 291 - DNA, 19 bp, Artificial
  siRNA target sequence
tctaaaagctcagcaaaag

SEQ ID NO: 292 - DNA, 19 bp, Artificial
  siRNA target sequence
ctaaaagctcagcaaaagc

SEQ ID NO: 293 - DNA, 19 bp, Artificial
  siRNA target sequence
taaaagctcagcaaaagct

SEQ ID NO: 294 - DNA, 19 bp, Artificial
  siRNA target sequence
aaaagctcagcaaaagcta

SEQ ID NO: 295 - DNA, 19 bp, Artificial
  siRNA target sequence
aaagctcagcaaaagctac

SEQ ID NO: 296 - DNA, 19 bp, Artificial
  siRNA target sequence
aagctcagcaaaagctacc

SEQ ID NO: 297 - DNA, 19 bp, Artificial
  siRNA target sequence
agctcagcaaaagctacca

SEQ ID NO: 298 - DNA, 19 bp, Artificial
  siRNA target sequence
gctcagcaaaagctaccac

SEQ ID NO: 299 - DNA, 19 bp, Artificial
  siRNA target sequence
ctcagcaaaagctaccacg

SEQ ID NO: 300 - DNA, 19 bp, Artificial
  siRNA target sequence
tcagcaaaagctaccacgc

SEQ ID NO: 301 - DNA, 19 bp, Artificial
  siRNA target sequence
cagcaaaagctaccacgct

SEQ ID NO: 302 - DNA, 19 bp, Artificial
  siRNA target sequence
agcaaaagctaccacgctt

SEQ ID NO: 303 - DNA, 19 bp, Artificial
  siRNA target sequence
gcaaaagctaccacgcttt

SEQ ID NO: 304 - DNA, 19 bp, Artificial
  siRNA target sequence
caaaagctaccacgctttc

SEQ ID NO: 305 - DNA, 19 bp, Artificial
  siRNA target sequence
aaaagctaccacgctttcc

SEQ ID NO: 306 - DNA, 19 bp, Artificial
  siRNA target sequence
aaagctaccacgctttcct

SEQ ID NO: 307 - DNA, 19 bp, Artificial
  siRNA target sequence
aagctaccacgctttcctt

SEQ ID NO: 308 - DNA, 19 bp, Artificial
  siRNA target sequence
agctaccacgctttccttg

SEQ ID NO: 309 - DNA, 19 bp, Artificial
  siRNA target sequence
gctaccacgctttccttgc

SEQ ID NO: 310 - DNA, 19 bp, Artificial
  siRNA target sequence
ctaccacgctttccttgcc

SEQ ID NO: 311 - DNA, 19 bp, Artificial
  siRNA target sequence
taccacgctttccttgcct

SEQ ID NO: 312 - DNA, 19 bp, Artificial
  siRNA target sequence
accacgctttccttgcctt

SEQ ID NO: 313 - DNA, 19 bp, Artificial
  siRNA target sequence
ccacgctttccttgccttc

SEQ ID NO: 314 - DNA, 19 bp, Artificial
  siRNA target sequence
cacgctttccttgccttcg

SEQ ID NO: 315 - DNA, 19 bp, Artificial
  siRNA target sequence
acgctttccttgccttcga

SEQ ID NO: 316 - DNA, 19 bp, Artificial
  siRNA target sequence
cgctttccttgccttcgaa

SEQ ID NO: 317 - DNA, 19 bp, Artificial
  siRNA target sequence
gctttccttgccttcgaat

SEQ ID NO: 318 - DNA, 19 bp, Artificial
  siRNA target sequence
ctttccttgccttcgaatc

SEQ ID NO: 319 - DNA, 19 bp, Artificial
  siRNA target sequence
tttccttgccttcgaatca

SEQ ID NO: 320 - DNA, 19 bp, Artificial
  siRNA target sequence
ttccttgccttcgaatcac

SEQ ID NO: 321 - DNA, 19 bp, Artificial
  siRNA target sequence
tccttgccttcgaatcact

SEQ ID NO: 322 - DNA, 19 bp, Artificial
  siRNA target sequence
ccttgccttcgaatcactc

SEQ ID NO: 323 - DNA, 19 bp, Artificial
  siRNA target sequence
cttgccttcgaatcactca

SEQ ID NO: 324 - DNA, 19 bp, Artificial
  siRNA target sequence
ttgccttcgaatcactcat

SEQ ID NO: 325 - DNA, 19 bp, Artificial
  siRNA target sequence
tgccttcgaatcactcatc

SEQ ID NO: 326 - DNA, 19 bp, Artificial
  siRNA target sequence
gccttcgaatcactcatca

SEQ ID NO: 327 - DNA, 19 bp, Artificial
  siRNA target sequence
ccttcgaatcactcatcaa

SEQ ID NO: 328 - DNA, 19 bp, Artificial
  siRNA target sequence
cttcgaatcactcatcaaa

SEQ ID NO: 329 - DNA, 19 bp, Artificial
  siRNA target sequence
ttcgaatcactcatcaaac

SEQ ID NO: 330 - DNA, 19 bp, Artificial
  siRNA target sequence
tcgaatcactcatcaaaca

SEQ ID NO: 331 - DNA, 19 bp, Artificial
  siRNA target sequence
cgaatcactcatcaaacaa

SEQ ID NO: 332 - DNA, 19 bp, Artificial
  siRNA target sequence
gaatcactcatcaaacaaa

SEQ ID NO: 333 - DNA, 19 bp, Artificial
  siRNA target sequence
aatcactcatcaaacaaat

SEQ ID NO: 334 - DNA, 19 bp, Artificial
  siRNA target sequence
atcactcatcaaacaaatc

SEQ ID NO: 335 - DNA, 19 bp, Artificial
  siRNA target sequence
tcactcatcaaacaaatcc

SEQ ID NO: 336 - DNA, 19 bp, Artificial
  siRNA target sequence
cactcatcaaacaaatccc

SEQ ID NO: 337 - DNA, 19 bp, Artificial
  siRNA target sequence
actcatcaaacaaatccct

SEQ ID NO: 338 - DNA, 19 bp, Artificial
  siRNA target sequence
ctcatcaaacaaatccctc

SEQ ID NO: 339 - DNA, 19 bp, Artificial
  siRNA target sequence
tcatcaaacaaatccctcg

SEQ ID NO: 340 - DNA, 19 bp, Artificial
  siRNA target sequence
catcaaacaaatccctcgt

SEQ ID NO: 341 - DNA, 19 bp, Artificial
  siRNA target sequence
atcaaacaaatccctcgta

SEQ ID NO: 342 - DNA, 19 bp, Artificial
  siRNA target sequence
tcaaacaaatccctcgtat

SEQ ID NO: 343 - DNA, 19 bp, Artificial
  siRNA target sequence
caaacaaatccctcgtatt

SEQ ID NO: 344 - DNA, 19 bp, Artificial
  siRNA target sequence
aaacaaatccctcgtattc

SEQ ID NO: 345 - DNA, 19 bp, Artificial
  siRNA target sequence
aacaaatccctcgtattct

SEQ ID NO: 346 - DNA, 19 bp, Artificial
  siRNA target sequence
acaaatccctcgtattctt

SEQ ID NO: 347 - DNA, 19 bp, Artificial
  siRNA target sequence
caaatccctcgtattcttg

SEQ ID NO: 348 - DNA, 19 bp, Artificial
  siRNA target sequence
aaatccctcgtattcttgg

SEQ ID NO: 349 - DNA, 19 bp, Artificial
  siRNA target sequence
aatccctcgtattcttggt

SEQ ID NO: 350 - DNA, 19 bp, Artificial
  siRNA target sequence
atccctcgtattcttggtc

SEQ ID NO: 351 - DNA, 19 bp, Artificial
  siRNA target sequence
tccctcgtattcttggtcc

SEQ ID NO: 352 - DNA, 19 bp, Artificial
  siRNA target sequence
ccctcgtattcttggtccc

SEQ ID NO: 353 - DNA, 19 bp, Artificial
  siRNA target sequence
cctcgtattcttggtcccg

SEQ ID NO: 354 - DNA, 19 bp, Artificial
  siRNA target sequence
ctcgtattcttggtcccgg

SEQ ID NO: 355 - DNA, 19 bp, Artificial
  siRNA target sequence
tcgtattcttggtcccgga

SEQ ID NO: 356 - DNA, 19 bp, Artificial
  siRNA target sequence
cgtattcttggtcccggac

SEQ ID NO: 357 - DNA, 19 bp, Artificial
  siRNA target sequence
gtattcttggtcccggact

SEQ ID NO: 358 - DNA, 19 bp, Artificial
  siRNA target sequence
tattcttggtcccggactg

SEQ ID NO: 359 - DNA, 19 bp, Artificial
  siRNA target sequence
attcttggtcccggactga

SEQ ID NO: 360 - DNA, 19 bp, Artificial
  siRNA target sequence
ttcttggtcccggactgaa

SEQ ID NO: 361 - DNA, 19 bp, Artificial
  siRNA target sequence
tcttggtcccggactgaac

SEQ ID NO: 362 - DNA, 19 bp, Artificial
  siRNA target sequence
cttggtcccggactgaaca

SEQ ID NO: 363 - DNA, 19 bp, Artificial
  siRNA target sequence
ttggtcccggactgaacaa

SEQ ID NO: 364 - DNA, 19 bp, Artificial
  siRNA target sequence
tggtcccggactgaacaag

SEQ ID NO: 365 - DNA, 19 bp, Artificial
  siRNA target sequence
ggtcccggactgaacaagg

SEQ ID NO: 366 - DNA, 19 bp, Artificial
  siRNA target sequence
gtcccggactgaacaaggc

SEQ ID NO: 367 - DNA, 19 bp, Artificial
  siRNA target sequence
tcccggactgaacaaggct

SEQ ID NO: 368 - DNA, 19 bp, Artificial
  siRNA target sequence
cccggactgaacaaggctg

SEQ ID NO: 369 - DNA, 19 bp, Artificial
  siRNA target sequence
ccggactgaacaaggctgg

SEQ ID NO: 370 - DNA, 19 bp, Artificial
  siRNA target sequence
cggactgaacaaggctggc

SEQ ID NO: 371 - DNA, 19 bp, Artificial
  siRNA target sequence
ggactgaacaaggctggca

SEQ ID NO: 372 - DNA, 19 bp, Artificial
  siRNA target sequence
gactgaacaaggctggcaa

SEQ ID NO: 373 - DNA, 19 bp, Artificial
  siRNA target sequence
actgaacaaggctggcaag

SEQ ID NO: 374 - DNA, 19 bp, Artificial
  siRNA target sequence
ctgaacaaggctggcaagt

SEQ ID NO: 375 - DNA, 19 bp, Artificial
  siRNA target sequence
tgaacaaggctggcaagtt

SEQ ID NO: 376 - DNA, 19 bp, Artificial
  siRNA target sequence
gaacaaggctggcaagttc

SEQ ID NO: 377 - DNA, 19 bp, Artificial
  siRNA target sequence
aacaaggctggcaagttcc

SEQ ID NO: 378 - DNA, 19 bp, Artificial
  siRNA target sequence
acaaggctggcaagttccc

SEQ ID NO: 379 - DNA, 19 bp, Artificial
  siRNA target sequence
caaggctggcaagttccca

SEQ ID NO: 380 - DNA, 19 bp, Artificial
  siRNA target sequence
aaggctggcaagttcccaa

SEQ ID NO: 381 - DNA, 19 bp, Artificial
  siRNA target sequence
aggctggcaagttcccaag

SEQ ID NO: 382 - DNA, 19 bp, Artificial
  siRNA target sequence
ggctggcaagttcccaagt

SEQ ID NO: 383 - DNA, 19 bp, Artificial
  siRNA target sequence
gctggcaagttcccaagtg

SEQ ID NO: 384 - DNA, 19 bp, Artificial
  siRNA target sequence
ctggcaagttcccaagtgt

SEQ ID NO: 385 - DNA, 19 bp, Artificial
  siRNA target sequence
tggcaagttcccaagtgtg

SEQ ID NO: 386 - DNA, 19 bp, Artificial
  siRNA target sequence
ggcaagttcccaagtgtgg

SEQ ID NO: 387 - DNA, 19 bp, Artificial
  siRNA target sequence
gcaagttcccaagtgtggt

SEQ ID NO: 388 - DNA, 19 bp, Artificial
  siRNA target sequence
caagttcccaagtgtggtg

SEQ ID NO: 389 - DNA, 19 bp, Artificial
  siRNA target sequence
aagttcccaagtgtggtgt

SEQ ID NO: 390 - DNA, 19 bp, Artificial
  siRNA target sequence
agttcccaagtgtggtgtc

SEQ ID NO: 391 - DNA, 19 bp, Artificial
  siRNA target sequence
gttcccaagtgtggtgtca

SEQ ID NO: 392 - DNA, 19 bp, Artificial
  siRNA target sequence
ttcccaagtgtggtgtcac

SEQ ID NO: 393 - DNA, 19 bp, Artificial
  siRNA target sequence
tcccaagtgtggtgtcaca

SEQ ID NO: 394 - DNA, 19 bp, Artificial
  siRNA target sequence
cccaagtgtggtgtcacac

SEQ ID NO: 395 - DNA, 19 bp, Artificial
  siRNA target sequence
ccaagtgtggtgtcacaca

SEQ ID NO: 396 - DNA, 19 bp, Artificial
  siRNA target sequence
caagtgtggtgtcacacaa

SEQ ID NO: 397 - DNA, 19 bp, Artificial
  siRNA target sequence
aagtgtggtgtcacacaac

SEQ ID NO: 398 - DNA, 19 bp, Artificial
  siRNA target sequence
agtgtggtgtcacacaacg

SEQ ID NO: 399 - DNA, 19 bp, Artificial
  siRNA target sequence
gtgtggtgtcacacaacga

SEQ ID NO: 400 - DNA, 19 bp, Artificial
  siRNA target sequence
tgtggtgtcacacaacgac

SEQ ID NO: 401 - DNA, 19 bp, Artificial
  siRNA target sequence
gtggtgtcacacaacgaca

SEQ ID NO: 402 - DNA, 19 bp, Artificial
  siRNA target sequence
tggtgtcacacaacgacat

SEQ ID NO: 403 - DNA, 19 bp, Artificial
  siRNA target sequence
ggtgtcacacaacgacatg

SEQ ID NO: 404 - DNA, 19 bp, Artificial
  siRNA target sequence
gtgtcacacaacgacatgc

SEQ ID NO: 405 - DNA, 19 bp, Artificial
  siRNA target sequence
tgtcacacaacgacatgct

SEQ ID NO: 406 - DNA, 19 bp, Artificial
  siRNA target sequence
gtcacacaacgacatgctg

SEQ ID NO: 407 - DNA, 19 bp, Artificial
  siRNA target sequence
tcacacaacgacatgctga

SEQ ID NO: 408 - DNA, 19 bp, Artificial
  siRNA target sequence
cacacaacgacatgctgaa

SEQ ID NO: 409 - DNA, 19 bp, Artificial
  siRNA target sequence
acacaacgacatgctgaac

SEQ ID NO: 410 - DNA, 19 bp, Artificial
  siRNA target sequence
cacaacgacatgctgaacg

SEQ ID NO: 411 - DNA, 19 bp, Artificial
  siRNA target sequence
acaacgacatgctgaacgc

SEQ ID NO: 412 - DNA, 19 bp, Artificial
  siRNA target sequence
caacgacatgctgaacgca

SEQ ID NO: 413 - DNA, 19 bp, Artificial
  siRNA target sequence
aacgacatgctgaacgcaa

SEQ ID NO: 414 - DNA, 19 bp, Artificial
  siRNA target sequence
acgacatgctgaacgcaaa

SEQ ID NO: 415 - DNA, 19 bp, Artificial
  siRNA target sequence
cgacatgctgaacgcaaag

SEQ ID NO: 416 - DNA, 19 bp, Artificial
  siRNA target sequence
gacatgctgaacgcaaagg

SEQ ID NO: 417 - DNA, 19 bp, Artificial
  siRNA target sequence
acatgctgaacgcaaaggt

SEQ ID NO: 418 - DNA, 19 bp, Artificial
  siRNA target sequence
catgctgaacgcaaaggtg

SEQ ID NO: 419 - DNA, 19 bp, Artificial
  siRNA target sequence
atgctgaacgcaaaggtgg

SEQ ID NO: 420 - DNA, 19 bp, Artificial
  siRNA target sequence
tgctgaacgcaaaggtgga

SEQ ID NO: 421 - DNA, 19 bp, Artificial
  siRNA target sequence
gctgaacgcaaaggtggat

SEQ ID NO: 422 - DNA, 19 bp, Artificial
  siRNA target sequence
ctgaacgcaaaggtggatg

SEQ ID NO: 423 - DNA, 19 bp, Artificial
  siRNA target sequence
tgaacgcaaaggtggatga

SEQ ID NO: 424 - DNA, 19 bp, Artificial
  siRNA target sequence
gaacgcaaaggtggatgaa

SEQ ID NO: 425 - DNA, 19 bp, Artificial
  siRNA target sequence
aacgcaaaggtggatgaag

SEQ ID NO: 426 - DNA, 19 bp, Artificial
  siRNA target sequence
acgcaaaggtggatgaagt

SEQ ID NO: 427 - DNA, 19 bp, Artificial
  siRNA target sequence
cgcaaaggtggatgaagtg

SEQ ID NO: 428 - DNA, 19 bp, Artificial
  siRNA target sequence
gcaaaggtggatgaagtga

SEQ ID NO: 429 - DNA, 19 bp, Artificial
  siRNA target sequence
caaaggtggatgaagtgaa

SEQ ID NO: 430 - DNA, 19 bp, Artificial
  siRNA target sequence
aaaggtggatgaagtgaag

SEQ ID NO: 431 - DNA, 19 bp, Artificial
  siRNA target sequence
aaggtggatgaagtgaagg

SEQ ID NO: 432 - DNA, 19 bp, Artificial
  siRNA target sequence
aggtggatgaagtgaaggc

SEQ ID NO: 433 - DNA, 19 bp, Artificial
  siRNA target sequence
ggtggatgaagtgaaggcg

SEQ ID NO: 434 - DNA, 19 bp, Artificial
  siRNA target sequence
gtggatgaagtgaaggcga

SEQ ID NO: 435 - DNA, 19 bp, Artificial
  siRNA target sequence
tggatgaagtgaaggcgaa

SEQ ID NO: 436 - DNA, 19 bp, Artificial
  siRNA target sequence
ggatgaagtgaaggcgaac

SEQ ID NO: 437 - DNA, 19 bp, Artificial
  siRNA target sequence
gatgaagtgaaggcgaacc

SEQ ID NO: 438 - DNA, 19 bp, Artificial
  siRNA target sequence
atgaagtgaaggcgaaccg

SEQ ID NO: 439 - DNA, 19 bp, Artificial
  siRNA target sequence
tgaagtgaaggcgaaccgc

SEQ ID NO: 440 - DNA, 19 bp, Artificial
  siRNA target sequence
gaagtgaaggcgaaccgca

SEQ ID NO: 441 - DNA, 19 bp, Artificial
  siRNA target sequence
aagtgaaggcgaaccgcaa

SEQ ID NO: 442 - DNA, 19 bp, Artificial
  siRNA target sequence
agtgaaggcgaaccgcaaa

SEQ ID NO: 443 - DNA, 19 bp, Artificial
  siRNA target sequence
gtgaaggcgaaccgcaaat

SEQ ID NO: 444 - DNA, 19 bp, Artificial
  siRNA target sequence
tgaaggcgaaccgcaaatt

SEQ ID NO: 445 - DNA, 19 bp, Artificial
  siRNA target sequence
gaaggcgaaccgcaaattc

SEQ ID NO: 446 - DNA, 19 bp, Artificial
  siRNA target sequence
aaggcgaaccgcaaattcg

SEQ ID NO: 447 - DNA, 19 bp, Artificial
  siRNA target sequence
aggcgaaccgcaaattcga

SEQ ID NO: 448 - DNA, 19 bp, Artificial
  siRNA target sequence
ggcgaaccgcaaattcgaa

SEQ ID NO: 449 - DNA, 19 bp, Artificial
  siRNA target sequence
gcgaaccgcaaattcgaaa

SEQ ID NO: 450 - DNA, 19 bp, Artificial
  siRNA target sequence
cgaaccgcaaattcgaaat

SEQ ID NO: 451 - DNA, 19 bp, Artificial
  siRNA target sequence
gaaccgcaaattcgaaatg

SEQ ID NO: 452 - DNA, 19 bp, Artificial
  siRNA target sequence
aaccgcaaattcgaaatga

SEQ ID NO: 453 - DNA, 19 bp, Artificial
  siRNA target sequence
accgcaaattcgaaatgaa

SEQ ID NO: 454 - DNA, 19 bp, Artificial
  siRNA target sequence
ccgcaaattcgaaatgaaa

SEQ ID NO: 455 - DNA, 19 bp, Artificial
  siRNA target sequence
cgcaaattcgaaatgaaac

SEQ ID NO: 456 - DNA, 19 bp, Artificial
  siRNA target sequence
gcaaattcgaaatgaaaca

SEQ ID NO: 457 - DNA, 19 bp, Artificial
  siRNA target sequence
caaattcgaaatgaaacag

SEQ ID NO: 458 - DNA, 19 bp, Artificial
  siRNA target sequence
aaattcgaaatgaaacagg

SEQ ID NO: 459 - DNA, 19 bp, Artificial
  siRNA target sequence
aattcgaaatgaaacaggt

SEQ ID NO: 460 - DNA, 19 bp, Artificial
  siRNA target sequence
attcgaaatgaaacaggtg

SEQ ID NO: 461 - DNA, 19 bp, Artificial
  siRNA target sequence
ttcgaaatgaaacaggtgc

SEQ ID NO: 462 - DNA, 19 bp, Artificial
  siRNA target sequence
tcgaaatgaaacaggtgct

SEQ ID NO: 463 - DNA, 19 bp, Artificial
  siRNA target sequence
cgaaatgaaacaggtgctc

SEQ ID NO: 464 - DNA, 19 bp, Artificial
  Antisense siRNA sequence
gagcaccuguuucauuucg

SEQ ID NO: 465 - DNA, 19 bp, Artificial
  Antisense siRNA sequence
agcaccuguuucauuucga

SEQ ID NO: 466 - DNA, 19 bp, Artificial
  Antisense siRNA sequence
gcaccuguuucauuucgaa

SEQ ID NO: 467 - DNA, 19 bp, Artificial
  Antisense siRNA sequence
caccuguuucauuucgaau

SEQ ID NO: 468 - DNA, 19 bp, Artificial
  Antisense siRNA sequence
accuguuucauuucgaauu

SEQ ID NO: 469 - DNA, 19 bp, Artificial
  Antisense siRNA sequence
ccuguuucauuucgaauuu

SEQ ID NO: 470 - DNA, 19 bp, Artificial
  Antisense siRNA sequence
cuguuucauuucgaauuug

SEQ ID NO: 471 - DNA, 19 bp, Artificial
  Antisense siRNA sequence
uguuucauuucgaauuugc

SEQ ID NO: 472 - DNA, 19 bp, Artificial
  Antisense siRNA sequence
guuucauuucgaauuugcg

SEQ ID NO: 473 - DNA, 19 bp, Artificial
  Antisense siRNA sequence
uuucauuucgaauuugcgg

SEQ ID NO: 474 - DNA, 19 bp, Artificial
  Antisense siRNA sequence
uucauuucgaauuugcggu

SEQ ID NO: 475 - DNA, 19 bp, Artificial
  Antisense siRNA sequence
ucauuucgaauuugcgguu

SEQ ID NO: 476 - DNA, 19 bp, Artificial
  Antisense siRNA sequence
cauuucgaauuugcgguuc

SEQ ID NO: 477 - DNA, 19 bp, Artificial
  Antisense siRNA sequence
auuucgaauuugcgguucg

SEQ ID NO: 478 - DNA, 19 bp, Artificial
  Antisense siRNA sequence
uuucgaauuugcgguucgc

SEQ ID NO: 479 - DNA, 19 bp, Artificial
  Antisense siRNA sequence
uucgaauuugcgguucgcc

SEQ ID NO: 480 - DNA, 19 bp, Artificial
  Antisense siRNA sequence
ucgaauuugcgguucgccu

SEQ ID NO: 481 - DNA, 19 bp, Artificial
  Antisense siRNA sequence
cgaauuugcgguucgccuu

SEQ ID NO: 482 - DNA, 19 bp, Artificial
  Antisense siRNA sequence
gaauuugcgguucgccuuc

SEQ ID NO: 483 - DNA, 19 bp, Artificial
  Antisense siRNA sequence
aauuugcgguucgccuuca

SEQ ID NO: 484 - DNA, 19 bp, Artificial
  Antisense siRNA sequence
auuugcgguucgccuucac

SEQ ID NO: 485 - DNA, 19 bp, Artificial
  Antisense siRNA sequence
uuugcgguucgccuucacu

SEQ ID NO: 486 - DNA, 19 bp, Artificial
  Antisense siRNA sequence
uugcgguucgccuucacuu

SEQ ID NO: 487 - DNA, 19 bp, Artificial
  Antisense siRNA sequence
ugcgguucgccuucacuuc

SEQ ID NO: 488 - DNA, 19 bp, Artificial
  Antisense siRNA sequence
gcgguucgccuucacuuca

SEQ ID NO: 489 - DNA, 19 bp, Artificial
  Antisense siRNA sequence
cgguucgccuucacuucau

SEQ ID NO: 490 - DNA, 19 bp, Artificial
  Antisense siRNA sequence
gguucgccuucacuucauc

SEQ ID NO: 491 - DNA, 19 bp, Artificial
  Antisense siRNA sequence
guucgccuucacuucaucc

SEQ ID NO: 492 - DNA, 19 bp, Artificial
  Antisense siRNA sequence
uucgccuucacuucaucca

SEQ ID NO: 493 - DNA, 19 bp, Artificial
  Antisense siRNA sequence
ucgccuucacuucauccac

SEQ ID NO: 494 - DNA, 19 bp, Artificial
  Antisense siRNA sequence
cgccuucacuucauccacc

SEQ ID NO: 495 - DNA, 19 bp, Artificial
  Antisense siRNA sequence
gccuucacuucauccaccu

SEQ ID NO: 496 - DNA, 19 bp, Artificial
  Antisense siRNA sequence
ccuucacuucauccaccuu

SEQ ID NO: 497 - DNA, 19 bp, Artificial
  Antisense siRNA sequence
cuucacuucauccaccuuu

SEQ ID NO: 498 - DNA, 19 bp, Artificial
  Antisense siRNA sequence
uucacuucauccaccuuug

SEQ ID NO: 499 - DNA, 19 bp, Artificial
  Antisense siRNA sequence
ucacuucauccaccuuugc

SEQ ID NO: 500 - DNA, 19 bp, Artificial
  Antisense siRNA sequence
cacuucauccaccuuugcg

SEQ ID NO: 501 - DNA, 19 bp, Artificial
  Antisense siRNA sequence
acuucauccaccuuugcgu

SEQ ID NO: 502 - DNA, 19 bp, Artificial
  Antisense siRNA sequence
cuucauccaccuuugcguu

SEQ ID NO: 503 - DNA, 19 bp, Artificial
  Antisense siRNA sequence
uucauccaccuuugcguuc

SEQ ID NO: 504 - DNA, 19 bp, Artificial
  Antisense siRNA sequence
ucauccaccuuugcguuca

SEQ ID NO: 505 - DNA, 19 bp, Artificial
  Antisense siRNA sequence
cauccaccuuugcguucag

SEQ ID NO: 506 - DNA, 19 bp, Artificial
  Antisense siRNA sequence
auccaccuuugcguucagc

SEQ ID NO: 507 - DNA, 19 bp, Artificial
  Antisense siRNA sequence
uccaccuuugcguucagca

SEQ ID NO: 508 - DNA, 19 bp, Artificial
  Antisense siRNA sequence
ccaccuuugcguucagcau

SEQ ID NO: 509 - DNA, 19 bp, Artificial
  Antisense siRNA sequence
caccuuugcguucagcaug

SEQ ID NO: 510 - DNA, 19 bp, Artificial
  Antisense siRNA sequence
accuuugcguucagcaugu

SEQ ID NO: 511 - DNA, 19 bp, Artificial
  Antisense siRNA sequence
ccuuugcguucagcauguc

SEQ ID NO: 512 - DNA, 19 bp, Artificial
  Antisense siRNA sequence
cuuugcguucagcaugucg

SEQ ID NO: 513 - DNA, 19 bp, Artificial
  Antisense siRNA sequence
uuugcguucagcaugucgu

SEQ ID NO: 514 - DNA, 19 bp, Artificial
  Antisense siRNA sequence
uugcguucagcaugucguu

SEQ ID NO: 515 - DNA, 19 bp, Artificial
  Antisense siRNA sequence
ugcguucagcaugucguug

SEQ ID NO: 516 - DNA, 19 bp, Artificial
  Antisense siRNA sequence
gcguucagcaugucguugu

SEQ ID NO: 517 - DNA, 19 bp, Artificial
  Antisense siRNA sequence
cguucagcaugucguugug

SEQ ID NO: 518 - DNA, 19 bp, Artificial
  Antisense siRNA sequence
guucagcaugucguugugu

SEQ ID NO: 519 - DNA, 19 bp, Artificial
  Antisense siRNA sequence
uucagcaugucguugugug

SEQ ID NO: 520 - DNA, 19 bp, Artificial
  Antisense siRNA sequence
ucagcaugucguuguguga

SEQ ID NO: 521 - DNA, 19 bp, Artificial
  Antisense siRNA sequence
cagcaugucguugugugac

SEQ ID NO: 522 - DNA, 19 bp, Artificial
  Antisense siRNA sequence
agcaugucguugugugaca

SEQ ID NO: 523 - DNA, 19 bp, Artificial
  Antisense siRNA sequence
gcaugucguugugugacac

SEQ ID NO: 524 - DNA, 19 bp, Artificial
  Antisense siRNA sequence
caugucguugugugacacc

SEQ ID NO: 525 - DNA, 19 bp, Artificial
  Antisense siRNA sequence
augucguugugugacacca

SEQ ID NO: 526 - DNA, 19 bp, Artificial
  Antisense siRNA sequence
ugucguugugugacaccac

SEQ ID NO: 527 - DNA, 19 bp, Artificial
  Antisense siRNA sequence
gucguugugugacaccaca

SEQ ID NO: 528 - DNA, 19 bp, Artificial
  Antisense siRNA sequence
ucguugugugacaccacac

SEQ ID NO: 529 - DNA, 19 bp, Artificial
  Antisense siRNA sequence
cguugugugacaccacacu

SEQ ID NO: 530 - DNA, 19 bp, Artificial
  Antisense siRNA sequence
guugugugacaccacacuu

SEQ ID NO: 531 - DNA, 19 bp, Artificial
  Antisense siRNA sequence
uugugugacaccacacuug

SEQ ID NO: 532 - DNA, 19 bp, Artificial
  Antisense siRNA sequence
ugugugacaccacacuugg

SEQ ID NO: 533 - DNA, 19 bp, Artificial
  Antisense siRNA sequence
gugugacaccacacuuggg

SEQ ID NO: 534 - DNA, 19 bp, Artificial
  Antisense siRNA sequence
ugugacaccacacuuggga

SEQ ID NO: 535 - DNA, 19 bp, Artificial
  Antisense siRNA sequence
gugacaccacacuugggaa

SEQ ID NO: 536 - DNA, 19 bp, Artificial
  Antisense siRNA sequence
ugacaccacacuugggaac

SEQ ID NO: 537 - DNA, 19 bp, Artificial
  Antisense siRNA sequence
gacaccacacuugggaacu

SEQ ID NO: 538 - DNA, 19 bp, Artificial
  Antisense siRNA sequence
acaccacacuugggaacuu

SEQ ID NO: 539 - DNA, 19 bp, Artificial
  Antisense siRNA sequence
caccacacuugggaacuug

SEQ ID NO: 540 - DNA, 19 bp, Artificial
  Antisense siRNA sequence
accacacuugggaacuugc

SEQ ID NO: 541 - DNA, 19 bp, Artificial
  Antisense siRNA sequence
ccacacuugggaacuugcc

SEQ ID NO: 542 - DNA, 19 bp, Artificial
  Antisense siRNA sequence
cacacuugggaacuugcca

SEQ ID NO: 543 - DNA, 19 bp, Artificial
  Antisense siRNA sequence
acacuugggaacuugccag

SEQ ID NO: 544 - DNA, 19 bp, Artificial
  Antisense siRNA sequence
cacuugggaacuugccagc

SEQ ID NO: 545 - DNA, 19 bp, Artificial
  Antisense siRNA sequence
acuugggaacuugccagcc

SEQ ID NO: 546 - DNA, 19 bp, Artificial
  Antisense siRNA sequence
cuugggaacuugccagccu

SEQ ID NO: 547 - DNA, 19 bp, Artificial
  Antisense siRNA sequence
uugggaacuugccagccuu

SEQ ID NO: 548 - DNA, 19 bp, Artificial
  Antisense siRNA sequence
ugggaacuugccagccuug

SEQ ID NO: 549 - DNA, 19 bp, Artificial
  Antisense siRNA sequence
gggaacuugccagccuugu

SEQ ID NO: 550 - DNA, 19 bp, Artificial
  Antisense siRNA sequence
ggaacuugccagccuuguu

SEQ ID NO: 551 - DNA, 19 bp, Artificial
  Antisense siRNA sequence
gaacuugccagccuuguuc

SEQ ID NO: 552 - DNA, 19 bp, Artificial
  Antisense siRNA sequence
aacuugccagccuuguuca

SEQ ID NO: 553 - DNA, 19 bp, Artificial
  Antisense siRNA sequence
acuugccagccuuguucag

SEQ ID NO: 554 - DNA, 19 bp, Artificial
  Antisense siRNA sequence
cuugccagccuuguucagu

SEQ ID NO: 555 - DNA, 19 bp, Artificial
  Antisense siRNA sequence
uugccagccuuguucaguc

SEQ ID NO: 556 - DNA, 19 bp, Artificial
  Antisense siRNA sequence
ugccagccuuguucagucc

SEQ ID NO: 557 - DNA, 19 bp, Artificial
  Antisense siRNA sequence
gccagccuuguucaguccg

SEQ ID NO: 558 - DNA, 19 bp, Artificial
  Antisense siRNA sequence
ccagccuuguucaguccgg

SEQ ID NO: 559 - DNA, 19 bp, Artificial
  Antisense siRNA sequence
cagccuuguucaguccggg

SEQ ID NO: 560 - DNA, 19 bp, Artificial
  Antisense siRNA sequence
agccuuguucaguccggga

SEQ ID NO: 561 - DNA, 19 bp, Artificial
  Antisense siRNA sequence
gccuuguucaguccgggac

SEQ ID NO: 562 - DNA, 19 bp, Artificial
  Antisense siRNA sequence
ccuuguucaguccgggacc

SEQ ID NO: 563 - DNA, 19 bp, Artificial
  Antisense siRNA sequence
cuuguucaguccgggacca

SEQ ID NO: 564 - DNA, 19 bp, Artificial
  Antisense siRNA sequence
uuguucaguccgggaccaa

SEQ ID NO: 565 - DNA, 19 bp, Artificial
  Antisense siRNA sequence
uguucaguccgggaccaag

SEQ ID NO: 566 - DNA, 19 bp, Artificial
  Antisense siRNA sequence
guucaguccgggaccaaga

SEQ ID NO: 567 - DNA, 19 bp, Artificial
  Antisense siRNA sequence
uucaguccgggaccaagaa

SEQ ID NO: 568 - DNA, 19 bp, Artificial
  Antisense siRNA sequence
ucaguccgggaccaagaau

SEQ ID NO: 569 - DNA, 19 bp, Artificial
  Antisense siRNA sequence
caguccgggaccaagaaua

SEQ ID NO: 570 - DNA, 19 bp, Artificial
  Antisense siRNA sequence
aguccgggaccaagaauac

SEQ ID NO: 571 - DNA, 19 bp, Artificial
  Antisense siRNA sequence
guccgggaccaagaauacg

SEQ ID NO: 572 - DNA, 19 bp, Artificial
  Antisense siRNA sequence
uccgggaccaagaauacga

SEQ ID NO: 573 - DNA, 19 bp, Artificial
  Antisense siRNA sequence
ccgggaccaagaauacgag

SEQ ID NO: 574 - DNA, 19 bp, Artificial
  Antisense siRNA sequence
cgggaccaagaauacgagg

SEQ ID NO: 575 - DNA, 19 bp, Artificial
  Antisense siRNA sequence
gggaccaagaauacgaggg

SEQ ID NO: 576 - DNA, 19 bp, Artificial
  Antisense siRNA sequence
ggaccaagaauacgaggga

SEQ ID NO: 577 - DNA, 19 bp, Artificial
  Antisense siRNA sequence
gaccaagaauacgagggau

SEQ ID NO: 578 - DNA, 19 bp, Artificial
  Antisense siRNA sequence
accaagaauacgagggauu

SEQ ID NO: 579 - DNA, 19 bp, Artificial
  Antisense siRNA sequence
ccaagaauacgagggauuu

SEQ ID NO: 580 - DNA, 19 bp, Artificial
  Antisense siRNA sequence
caagaauacgagggauuug

SEQ ID NO: 581 - DNA, 19 bp, Artificial
  Antisense siRNA sequence
aagaauacgagggauuugu

SEQ ID NO: 582 - DNA, 19 bp, Artificial
  Antisense siRNA sequence
agaauacgagggauuuguu

SEQ ID NO: 583 - DNA, 19 bp, Artificial
  Antisense siRNA sequence
gaauacgagggauuuguuu

SEQ ID NO: 584 - DNA, 19 bp, Artificial
  Antisense siRNA sequence
aauacgagggauuuguuug

SEQ ID NO: 585 - DNA, 19 bp, Artificial
  Antisense siRNA sequence
auacgagggauuuguuuga

SEQ ID NO: 586 - DNA, 19 bp, Artificial
  Antisense siRNA sequence
uacgagggauuuguuugau

SEQ ID NO: 587 - DNA, 19 bp, Artificial
  Antisense siRNA sequence
acgagggauuuguuugaug

SEQ ID NO: 588 - DNA, 19 bp, Artificial
  Antisense siRNA sequence
cgagggauuuguuugauga

SEQ ID NO: 589 - DNA, 19 bp, Artificial
  Antisense siRNA sequence
gagggauuuguuugaugag

SEQ ID NO: 590 - DNA, 19 bp, Artificial
  Antisense siRNA sequence
agggauuuguuugaugagu

SEQ ID NO: 591 - DNA, 19 bp, Artificial
  Antisense siRNA sequence
gggauuuguuugaugagug

SEQ ID NO: 592 - DNA, 19 bp, Artificial
  Antisense siRNA sequence
ggauuuguuugaugaguga

SEQ ID NO: 593 - DNA, 19 bp, Artificial
  Antisense siRNA sequence
gauuuguuugaugagugau

SEQ ID NO: 594 - DNA, 19 bp, Artificial
  Antisense siRNA sequence
auuuguuugaugagugauu

SEQ ID NO: 595 - DNA, 19 bp, Artificial
  Antisense siRNA sequence
uuuguuugaugagugauuc

SEQ ID NO: 596 - DNA, 19 bp, Artificial
  Antisense siRNA sequence
uuguuugaugagugauucg

SEQ ID NO: 597 - DNA, 19 bp, Artificial
  Antisense siRNA sequence
uguuugaugagugauucga

SEQ ID NO: 598 - DNA, 19 bp, Artificial
  Antisense siRNA sequence
guuugaugagugauucgaa

SEQ ID NO: 599 - DNA, 19 bp, Artificial
  Antisense siRNA sequence
uuugaugagugauucgaag

SEQ ID NO: 600 - DNA, 19 bp, Artificial
  Antisense siRNA sequence
uugaugagugauucgaagg

SEQ ID NO: 601 - DNA, 19 bp, Artificial
  Antisense siRNA sequence
ugaugagugauucgaaggc

SEQ ID NO: 602 - DNA, 19 bp, Artificial
  Antisense siRNA sequence
gaugagugauucgaaggca

SEQ ID NO: 603 - DNA, 19 bp, Artificial
  Antisense siRNA sequence
augagugauucgaaggcaa

SEQ ID NO: 604 - DNA, 19 bp, Artificial
  Antisense siRNA sequence
ugagugauucgaaggcaag

SEQ ID NO: 605 - DNA, 19 bp, Artificial
  Antisense siRNA sequence
gagugauucgaaggcaagg

SEQ ID NO: 606 - DNA, 19 bp, Artificial
  Antisense siRNA sequence
agugauucgaaggcaagga

SEQ ID NO: 607 - DNA, 19 bp, Artificial
  Antisense siRNA sequence
gugauucgaaggcaaggaa

SEQ ID NO: 608 - DNA, 19 bp, Artificial
  Antisense siRNA sequence
ugauucgaaggcaaggaaa

SEQ ID NO: 609 - DNA, 19 bp, Artificial
  Antisense siRNA sequence
gauucgaaggcaaggaaag

SEQ ID NO: 610 - DNA, 19 bp, Artificial
  Antisense siRNA sequence
auucgaaggcaaggaaagc

SEQ ID NO: 611 - DNA, 19 bp, Artificial
  Antisense siRNA sequence
uucgaaggcaaggaaagcg

SEQ ID NO: 612 - DNA, 19 bp, Artificial
  Antisense siRNA sequence
ucgaaggcaaggaaagcgu

SEQ ID NO: 613 - DNA, 19 bp, Artificial
  Antisense siRNA sequence
cgaaggcaaggaaagcgug

SEQ ID NO: 614 - DNA, 19 bp, Artificial
  Antisense siRNA sequence
gaaggcaaggaaagcgugg

SEQ ID NO: 615 - DNA, 19 bp, Artificial
  Antisense siRNA sequence
aaggcaaggaaagcguggu

SEQ ID NO: 616 - DNA, 19 bp, Artificial
  Antisense siRNA sequence
aggcaaggaaagcguggua

SEQ ID NO: 617 - DNA, 19 bp, Artificial
  Antisense siRNA sequence
ggcaaggaaagcgugguag

SEQ ID NO: 618 - DNA, 19 bp, Artificial
  Antisense siRNA sequence
gcaaggaaagcgugguagc

SEQ ID NO: 619 - DNA, 19 bp, Artificial
  Antisense siRNA sequence
caaggaaagcgugguagcu

SEQ ID NO: 620 - DNA, 19 bp, Artificial
  Antisense siRNA sequence
aaggaaagcgugguagcuu

SEQ ID NO: 621 - DNA, 19 bp, Artificial
  Antisense siRNA sequence
aggaaagcgugguagcuuu

SEQ ID NO: 622 - DNA, 19 bp, Artificial
  Antisense siRNA sequence
ggaaagcgugguagcuuuu

SEQ ID NO: 623 - DNA, 19 bp, Artificial
  Antisense siRNA sequence
gaaagcgugguagcuuuug

SEQ ID NO: 624 - DNA, 19 bp, Artificial
  Antisense siRNA sequence
aaagcgugguagcuuuugc

SEQ ID NO: 625 - DNA, 19 bp, Artificial
  Antisense siRNA sequence
aagcgugguagcuuuugcu

SEQ ID NO: 626 - DNA, 19 bp, Artificial
  Antisense siRNA sequence
agcgugguagcuuuugcug

SEQ ID NO: 627 - DNA, 19 bp, Artificial
  Antisense siRNA sequence
gcgugguagcuuuugcuga

SEQ ID NO: 628 - DNA, 19 bp, Artificial
  Antisense siRNA sequence
cgugguagcuuuugcugag

SEQ ID NO: 629 - DNA, 19 bp, Artificial
  Antisense siRNA sequence
gugguagcuuuugcugagc

SEQ ID NO: 630 - DNA, 19 bp, Artificial
  Antisense siRNA sequence
ugguagcuuuugcugagcu

SEQ ID NO: 631 - DNA, 19 bp, Artificial
  Antisense siRNA sequence
gguagcuuuugcugagcuu

SEQ ID NO: 632 - DNA, 19 bp, Artificial
  Antisense siRNA sequence
guagcuuuugcugagcuuu

SEQ ID NO: 633 - DNA, 19 bp, Artificial
  Antisense siRNA sequence
uagcuuuugcugagcuuuu

SEQ ID NO: 634 - DNA, 19 bp, Artificial
  Antisense siRNA sequence
agcuuuugcugagcuuuua

SEQ ID NO: 635 - DNA, 19 bp, Artificial
  Antisense siRNA sequence
gcuuuugcugagcuuuuag

SEQ ID NO: 636 - DNA, 19 bp, Artificial
  Antisense siRNA sequence
cuuuugcugagcuuuuaga

SEQ ID NO: 637 - DNA, 19 bp, Artificial
  Antisense siRNA sequence
uuuugcugagcuuuuagau

SEQ ID NO: 638 - DNA, 19 bp, Artificial
  Antisense siRNA sequence
uuugcugagcuuuuagauc

SEQ ID NO: 639 - DNA, 19 bp, Artificial
  Antisense siRNA sequence
uugcugagcuuuuagauca

SEQ ID NO: 640 - DNA, 19 bp, Artificial
  Antisense siRNA sequence
ugcugagcuuuuagaucag

SEQ ID NO: 641 - DNA, 19 bp, Artificial
  Antisense siRNA sequence
gcugagcuuuuagaucagc

SEQ ID NO: 642 - DNA, 19 bp, Artificial
  Antisense siRNA sequence
cugagcuuuuagaucagcu

SEQ ID NO: 643 - DNA, 19 bp, Artificial
  Antisense siRNA sequence
ugagcuuuuagaucagcuu

SEQ ID NO: 644 - DNA, 19 bp, Artificial
  Antisense siRNA sequence
gagcuuuuagaucagcuuc

SEQ ID NO: 645 - DNA, 19 bp, Artificial
  Antisense siRNA sequence
agcuuuuagaucagcuucu

SEQ ID NO: 646 - DNA, 19 bp, Artificial
  Antisense siRNA sequence
gcuuuuagaucagcuucuu

SEQ ID NO: 647 - DNA, 19 bp, Artificial
  Antisense siRNA sequence
cuuuuagaucagcuucuug

SEQ ID NO: 648 - DNA, 19 bp, Artificial
  Antisense siRNA sequence
uuuuagaucagcuucuugu

SEQ ID NO: 649 - DNA, 19 bp, Artificial
  Antisense siRNA sequence
uuuagaucagcuucuuguc

SEQ ID NO: 650 - DNA, 19 bp, Artificial
  Antisense siRNA sequence
uuagaucagcuucuugucc

SEQ ID NO: 651 - DNA, 19 bp, Artificial
  Antisense siRNA sequence
uagaucagcuucuuguccu

SEQ ID NO: 652 - DNA, 19 bp, Artificial
  Antisense siRNA sequence
agaucagcuucuuguccuu

SEQ ID NO: 653 - DNA, 19 bp, Artificial
  Antisense siRNA sequence
gaucagcuucuuguccuug

SEQ ID NO: 654 - DNA, 19 bp, Artificial
  Antisense siRNA sequence
aucagcuucuuguccuugu

SEQ ID NO: 655 - DNA, 19 bp, Artificial
  Antisense siRNA sequence
ucagcuucuuguccuuguu

SEQ ID NO: 656 - DNA, 19 bp, Artificial
  Antisense siRNA sequence
cagcuucuuguccuuguuc

SEQ ID NO: 657 - DNA, 19 bp, Artificial
  Antisense siRNA sequence
agcuucuuguccuuguuca

SEQ ID NO: 658 - DNA, 19 bp, Artificial
  Antisense siRNA sequence
gcuucuuguccuuguucaa

SEQ ID NO: 659 - DNA, 19 bp, Artificial
  Antisense siRNA sequence
cuucuuguccuuguucaac

SEQ ID NO: 660 - DNA, 19 bp, Artificial
  Antisense siRNA sequence
uucuuguccuuguucaacu

SEQ ID NO: 661 - DNA, 19 bp, Artificial
  Antisense siRNA sequence
ucuuguccuuguucaacuu

SEQ ID NO: 662 - DNA, 19 bp, Artificial
  Antisense siRNA sequence
cuuguccuuguucaacuuc

SEQ ID NO: 663 - DNA, 19 bp, Artificial
  Antisense siRNA sequence
uuguccuuguucaacuucu

SEQ ID NO: 664 - DNA, 19 bp, Artificial
  Antisense siRNA sequence
uguccuuguucaacuucuu

SEQ ID NO: 665 - DNA, 19 bp, Artificial
  Antisense siRNA sequence
guccuuguucaacuucuuc

SEQ ID NO: 666 - DNA, 19 bp, Artificial
  Antisense siRNA sequence
uccuuguucaacuucuuca

SEQ ID NO: 667 - DNA, 19 bp, Artificial
  Antisense siRNA sequence
ccuuguucaacuucuucag

SEQ ID NO: 668 - DNA, 19 bp, Artificial
  Antisense siRNA sequence
cuuguucaacuucuucagg

SEQ ID NO: 669 - DNA, 19 bp, Artificial
  Antisense siRNA sequence
uuguucaacuucuucaggu

SEQ ID NO: 670 - DNA, 19 bp, Artificial
  Antisense siRNA sequence
uguucaacuucuucagguc

SEQ ID NO: 671 - DNA, 19 bp, Artificial
  Antisense siRNA sequence
guucaacuucuucaggucg

SEQ ID NO: 672 - DNA, 19 bp, Artificial
  Antisense siRNA sequence
uucaacuucuucaggucgu

SEQ ID NO: 673 - DNA, 19 bp, Artificial
  Antisense siRNA sequence
ucaacuucuucaggucguc

SEQ ID NO: 674 - DNA, 19 bp, Artificial
  Antisense siRNA sequence
caacuucuucaggucgucc

SEQ ID NO: 675 - DNA, 19 bp, Artificial
  Antisense siRNA sequence
aacuucuucaggucguccg

SEQ ID NO: 676 - DNA, 19 bp, Artificial
  Antisense siRNA sequence
acuucuucaggucguccgc

SEQ ID NO: 677 - DNA, 19 bp, Artificial
  Antisense siRNA sequence
cuucuucaggucguccgcu

SEQ ID NO: 678 - DNA, 19 bp, Artificial
  Antisense siRNA sequence
uucuucaggucguccgcug

SEQ ID NO: 679 - DNA, 19 bp, Artificial
  Antisense siRNA sequence
ucuucaggucguccgcugu

SEQ ID NO: 680 - DNA, 19 bp, Artificial
  Antisense siRNA sequence
cuucaggucguccgcuguc

SEQ ID NO: 681 - DNA, 19 bp, Artificial
  Antisense siRNA sequence
uucaggucguccgcuguca

SEQ ID NO: 682 - DNA, 19 bp, Artificial
  Antisense siRNA sequence
ucaggucguccgcugucau

SEQ ID NO: 683 - DNA, 19 bp, Artificial
  Antisense siRNA sequence
caggucguccgcugucaug

SEQ ID NO: 684 - DNA, 19 bp, Artificial
  Antisense siRNA sequence
aggucguccgcugucaugc

SEQ ID NO: 685 - DNA, 19 bp, Artificial
  Antisense siRNA sequence
gucguccgcugucaugcau

SEQ ID NO: 686 - DNA, 19 bp, Artificial
  Antisense siRNA sequence
gucguccgcugucaugcau

SEQ ID NO: 687 - DNA, 19 bp, Artificial
  Antisense siRNA sequence
ucguccgcugucaugcaug

SEQ ID NO: 688 - DNA, 19 bp, Artificial
  Antisense siRNA sequence
cguccgcugucaugcaugg

SEQ ID NO: 689 - DNA, 19 bp, Artificial
  Antisense siRNA sequence
guccgcugucaugcaugga

SEQ ID NO: 690 - DNA, 19 bp, Artificial
  Antisense siRNA sequence
uccgcugucaugcauggaa

SEQ ID NO: 691 - DNA, 19 bp, Artificial
  Antisense siRNA sequence
ccgcugucaugcauggaau

SEQ ID NO: 692 - DNA, 19 bp, Artificial
  Antisense siRNA sequence
cgcugucaugcauggaauu

SEQ ID NO: 693 - DNA, 19 bp, Artificial
  Antisense siRNA sequence
gcugucaugcauggaauuc

SEQ ID NO: 694 - DNA, 19 bp, Artificial
  Antisense siRNA sequence
cugucaugcauggaauucc

SEQ ID NO: 695 - DNA, 19 bp, Artificial
  Antisense siRNA sequence
ugucaugcauggaauuccg

SEQ ID NO: 696 - DNA, 19 bp, Artificial
  Antisense siRNA sequence
gucaugcauggaauuccgu

SEQ ID NO: 697 - DNA, 19 bp, Artificial
  Antisense siRNA sequence
ucaugcauggaauuccguu

SEQ ID NO: 698 - DNA, 19 bp, Artificial
  Antisense siRNA sequence
caugcauggaauuccguug

SEQ ID NO: 699 - DNA, 19 bp, Artificial
  Antisense siRNA sequence
augcauggaauuccguugg

SEQ ID NO: 700 - DNA, 19 bp, Artificial
  Antisense siRNA sequence
ugcauggaauuccguuggc

SEQ ID NO: 701 - DNA, 19 bp, Artificial
  Antisense siRNA sequence
gcauggaauuccguuggca

SEQ ID NO: 702 - DNA, 19 bp, Artificial
  Antisense siRNA sequence
cauggaauuccguuggcau

SEQ ID NO: 703 - DNA, 19 bp, Artificial
  Antisense siRNA sequence
auggaauuccguuggcauu

SEQ ID NO: 704 - DNA, 19 bp, Artificial
  Antisense siRNA sequence
uggaauuccguuggcauug

SEQ ID NO: 705 - DNA, 19 bp, Artificial
  Antisense siRNA sequence
ggaauuccguuggcauugg

SEQ ID NO: 706 - DNA, 19 bp, Artificial
  Antisense siRNA sequence
gaauuccguuggcauuggc

SEQ ID NO: 707 - DNA, 19 bp, Artificial
  Antisense siRNA sequence
aauuccguuggcauuggcc

SEQ ID NO: 708 - DNA, 19 bp, Artificial
  Antisense siRNA sequence
auuccguuggcauuggccu

SEQ ID NO: 709 - DNA, 19 bp, Artificial
  Antisense siRNA sequence
uuccguuggcauuggccuc

SEQ ID NO: 710 - DNA, 19 bp, Artificial
  Antisense siRNA sequence
uccguuggcauuggccucg

SEQ ID NO: 711 - DNA, 19 bp, Artificial
  Antisense siRNA sequence
ccguuggcauuggccucgu

SEQ ID NO: 712 - DNA, 19 bp, Artificial
  Antisense siRNA sequence
cguuggcauuggccucguc

SEQ ID NO: 713 - DNA, 19 bp, Artificial
  Antisense siRNA sequence
guuggcauuggccucguca

SEQ ID NO: 714 - DNA, 19 bp, Artificial
  Antisense siRNA sequence
uuggcauuggccucgucac

SEQ ID NO: 715 - DNA, 19 bp, Artificial
  Antisense siRNA sequence
uggcauuggccucgucaca

SEQ ID NO: 716 - DNA, 19 bp, Artificial
  Antisense siRNA sequence
ggcauuggccucgucacaa

SEQ ID NO: 717 - DNA, 19 bp, Artificial
  Antisense siRNA sequence
gcauuggccucgucacaau

SEQ ID NO: 718 - DNA, 19 bp, Artificial
  Antisense siRNA sequence
cauuggccucgucacaaug

SEQ ID NO: 719 - DNA, 19 bp, Artificial
  Antisense siRNA sequence
auuggccucgucacaaugu

SEQ ID NO: 720 - DNA, 19 bp, Artificial
  Antisense siRNA sequence
uuggccucgucacaauguu

SEQ ID NO: 721 - DNA, 19 bp, Artificial
  Antisense siRNA sequence
uggccucgucacaauguuu

SEQ ID NO: 722 - DNA, 19 bp, Artificial
  Antisense siRNA sequence
ggccucgucacaauguuuu

SEQ ID NO: 723 - DNA, 19 bp, Artificial
  Antisense siRNA sequence
gccucgucacaauguuuuu

SEQ ID NO: 724 - DNA, 19 bp, Artificial
  Antisense siRNA sequence
ccucgucacaauguuuuug

SEQ ID NO: 725 - DNA, 19 bp, Artificial
  Antisense siRNA sequence
cucgucacaauguuuuugg

SEQ ID NO: 726 - DNA, 19 bp, Artificial
  Antisense siRNA sequence
ucgucacaauguuuuuggu

SEQ ID NO: 727 - DNA, 19 bp, Artificial
  Antisense siRNA sequence
cgucacaauguuuuugguc

SEQ ID NO: 728 - DNA, 19 bp, Artificial
  Antisense siRNA sequence
gucacaauguuuuuggucg

SEQ ID NO: 729 - DNA, 19 bp, Artificial
  Antisense siRNA sequence
ucacaauguuuuuggucgc

SEQ ID NO: 730 - DNA, 19 bp, Artificial
  Antisense siRNA sequence
cacaauguuuuuggucgcc

SEQ ID NO: 731 - DNA, 19 bp, Artificial
  Antisense siRNA sequence
acaauguuuuuggucgcca

SEQ ID NO: 732 - DNA, 19 bp, Artificial
  Antisense siRNA sequence
caauguuuuuggucgccaa

SEQ ID NO: 733 - DNA, 19 bp, Artificial
  Antisense siRNA sequence
aauguuuuuggucgccaag

SEQ ID NO: 734 - DNA, 19 bp, Artificial
  Antisense siRNA sequence
auguuuuuggucgccaagg

SEQ ID NO: 735 - DNA, 19 bp, Artificial
  Antisense siRNA sequence
uguuuuuggucgccaagga

SEQ ID NO: 736 - DNA, 19 bp, Artificial
  Antisense siRNA sequence
guuuuuggucgccaaggau

SEQ ID NO: 737 - DNA, 19 bp, Artificial
  Antisense siRNA sequence
uuuuuggucgccaaggaug

SEQ ID NO: 738 - DNA, 19 bp, Artificial
  Antisense siRNA sequence
uuuuggucgccaaggaugc

SEQ ID NO: 739 - DNA, 19 bp, Artificial
  Antisense siRNA sequence
uuuggucgccaaggaugca

SEQ ID NO: 740 - DNA, 19 bp, Artificial
  Antisense siRNA sequence
uuggucgccaaggaugcaa

SEQ ID NO: 741 - DNA, 19 bp, Artificial
  Antisense siRNA sequence
uggucgccaaggaugcaaa

SEQ ID NO: 742 - DNA, 19 bp, Artificial
  Antisense siRNA sequence
ggucgccaaggaugcaaac

SEQ ID NO: 743 - DNA, 19 bp, Artificial
  Antisense siRNA sequence
gucgccaaggaugcaaacc

SEQ ID NO: 744 - DNA, 19 bp, Artificial
  Antisense siRNA sequence
ucgccaaggaugcaaaccu

SEQ ID NO: 745 - DNA, 19 bp, Artificial
  Antisense siRNA sequence
cgccaaggaugcaaaccuu

SEQ ID NO: 746 - DNA, 19 bp, Artificial
  Antisense siRNA sequence
gccaaggaugcaaaccuuc

SEQ ID NO: 747 - DNA, 19 bp, Artificial
  Antisense siRNA sequence
ccaaggaugcaaaccuucg

SEQ ID NO: 748 - DNA, 19 bp, Artificial
  Antisense siRNA sequence
caaggaugcaaaccuucgu

SEQ ID NO: 749 - DNA, 19 bp, Artificial
  Antisense siRNA sequence
aaggaugcaaaccuucguu

SEQ ID NO: 750 - DNA, 19 bp, Artificial
  Antisense siRNA sequence
aggaugcaaaccuucguuu

SEQ ID NO: 751 - DNA, 19 bp, Artificial
  Antisense siRNA sequence
ggaugcaaaccuucguuuu

SEQ ID NO: 752 - DNA, 19 bp, Artificial
  Antisense siRNA sequence
gaugcaaaccuucguuuuc

SEQ ID NO: 753 - DNA, 19 bp, Artificial
  Antisense siRNA sequence
augcaaaccuucguuuucg

SEQ ID NO: 754 - DNA, 19 bp, Artificial
  Antisense siRNA sequence
ugcaaaccuucguuuucgg

SEQ ID NO: 755 - DNA, 19 bp, Artificial
  Antisense siRNA sequence
gcaaaccuucguuuucgga

SEQ ID NO: 756 - DNA, 19 bp, Artificial
  Antisense siRNA sequence
caaaccuucguuuucggac

SEQ ID NO: 757 - DNA, 19 bp, Artificial
  Antisense siRNA sequence
aaaccuucguuuucggacg

SEQ ID NO: 758 - DNA, 19 bp, Artificial
  Antisense siRNA sequence
aaccuucguuuucggacga

SEQ ID NO: 759 - DNA, 19 bp, Artificial
  Antisense siRNA sequence
accuucguuuucggacgag

SEQ ID NO: 760 - DNA, 19 bp, Artificial
  Antisense siRNA sequence
ccuucguuuucggacgagg

SEQ ID NO: 761 - DNA, 19 bp, Artificial
  Antisense siRNA sequence
cuucguuuucggacgaggg

SEQ ID NO: 762 - DNA, 19 bp, Artificial
  Antisense siRNA sequence
uucguuuucggacgaggga

SEQ ID NO: 763 - DNA, 19 bp, Artificial
  Antisense siRNA sequence
ucguuuucggacgagggau

SEQ ID NO: 764 - DNA, 19 bp, Artificial
  Antisense siRNA sequence
cguuuucggacgagggaug

SEQ ID NO: 765 - DNA, 19 bp, Artificial
  Antisense siRNA sequence
guuuucggacgagggaugu

SEQ ID NO: 766 - DNA, 19 bp, Artificial
  Antisense siRNA sequence
uuuucggacgagggaugug

SEQ ID NO: 767 - DNA, 19 bp, Artificial
  Antisense siRNA sequence
uuucggacgagggaugugc

SEQ ID NO: 768 - DNA, 19 bp, Artificial
  Antisense siRNA sequence
uucggacgagggaugugcu

SEQ ID NO: 769 - DNA, 19 bp, Artificial
  Antisense siRNA sequence
ucggacgagggaugugcuu

SEQ ID NO: 770 - DNA, 19 bp, Artificial
  Antisense siRNA sequence
cggacgagggaugugcuuc

SEQ ID NO: 771 - DNA, 19 bp, Artificial
  Antisense siRNA sequence
ggacgagggaugugcuuca

SEQ ID NO: 772 - DNA, 19 bp, Artificial
  Antisense siRNA sequence
gacgagggaugugcuucag

SEQ ID NO: 773 - DNA, 19 bp, Artificial
  Antisense siRNA sequence
acgagggaugugcuucagu

SEQ ID NO: 774 - DNA, 19 bp, Artificial
  Antisense siRNA sequence
cgagggaugugcuucaguc

SEQ ID NO: 775 - DNA, 19 bp, Artificial
  Antisense siRNA sequence
gagggaugugcuucagucu

SEQ ID NO: 776 - DNA, 19 bp, Artificial
  Antisense siRNA sequence
agggaugugcuucagucua

SEQ ID NO: 777 - DNA, 19 bp, Artificial
  Antisense siRNA sequence
gggaugugcuucagucuaa

SEQ ID NO: 778 - DNA, 19 bp, Artificial
  Antisense siRNA sequence
ggaugugcuucagucuaac

SEQ ID NO: 779 - DNA, 19 bp, Artificial
  Antisense siRNA sequence
gaugugcuucagucuaaca

SEQ ID NO: 780 - DNA, 19 bp, Artificial
  Antisense siRNA sequence
augugcuucagucuaacag

SEQ ID NO: 781 - DNA, 19 bp, Artificial
  Antisense siRNA sequence
ugugcuucagucuaacagu

SEQ ID NO: 782 - DNA, 19 bp, Artificial
  Antisense siRNA sequence
gugcuucagucuaacaguu

SEQ ID NO: 783 - DNA, 19 bp, Artificial
  Antisense siRNA sequence
ugcuucagucuaacaguuc

SEQ ID NO: 784 - DNA, 19 bp, Artificial
  Antisense siRNA sequence
gcuucagucuaacaguucc

SEQ ID NO: 785 - DNA, 19 bp, Artificial
  Antisense siRNA sequence
cuucagucuaacaguucca

SEQ ID NO: 786 - DNA, 19 bp, Artificial
  Antisense siRNA sequence
uucagucuaacaguuccac

SEQ ID NO: 787 - DNA, 19 bp, Artificial
  Antisense siRNA sequence
ucagucuaacaguuccacu

SEQ ID NO: 788 - DNA, 19 bp, Artificial
  Antisense siRNA sequence
cagucuaacaguuccacug

SEQ ID NO: 789 - DNA, 19 bp, Artificial
  Antisense siRNA sequence
agucuaacaguuccacuga

SEQ ID NO: 790 - DNA, 19 bp, Artificial
  Antisense siRNA sequence
gucuaacaguuccacugaa

SEQ ID NO: 791 - DNA, 19 bp, Artificial
  Antisense siRNA sequence
ucuaacaguuccacugaaa

SEQ ID NO: 792 - DNA, 19 bp, Artificial
  Antisense siRNA sequence
cuaacaguuccacugaaac

SEQ ID NO: 793 - DNA, 19 bp, Artificial
  Antisense siRNA sequence
uaacaguuccacugaaacg

SEQ ID NO: 794 - DNA, 19 bp, Artificial
  Antisense siRNA sequence
aacaguuccacugaaacgc

SEQ ID NO: 795 - DNA, 19 bp, Artificial
  Antisense siRNA sequence
acaguuccacugaaacgcu

SEQ ID NO: 796 - DNA, 19 bp, Artificial
  Antisense siRNA sequence
caguuccacugaaacgcuu

SEQ ID NO: 797 - DNA, 19 bp, Artificial
  Antisense siRNA sequence
aguuccacugaaacgcuug

SEQ ID NO: 798 - DNA, 19 bp, Artificial
  Antisense siRNA sequence
guuccacugaaacgcuugu

SEQ ID NO: 799 - DNA, 19 bp, Artificial
  Antisense siRNA sequence
uuccacugaaacgcuuguc

SEQ ID NO: 800 - DNA, 19 bp, Artificial
  Antisense siRNA sequence
uccacugaaacgcuugucc

SEQ ID NO: 801 - DNA, 19 bp, Artificial
  Antisense siRNA sequence
ccacugaaacgcuuguccu

SEQ ID NO: 802 - DNA, 19 bp, Artificial
  Antisense siRNA sequence
cacugaaacgcuuguccuu

SEQ ID NO: 803 - DNA, 19 bp, Artificial
  Antisense siRNA sequence
acugaaacgcuuguccuuc

SEQ ID NO: 804 - DNA, 19 bp, Artificial
  Antisense siRNA sequence
cugaaacgcuuguccuucu

SEQ ID NO: 805 - DNA, 19 bp, Artificial
  Antisense siRNA sequence
ugaaacgcuuguccuucug

SEQ ID NO: 806 - DNA, 19 bp, Artificial
  Antisense siRNA sequence
gaaacgcuuguccuucugu

SEQ ID NO: 807 - DNA, 19 bp, Artificial
  Antisense siRNA sequence
aaacgcuuguccuucugug

SEQ ID NO: 808 - DNA, 19 bp, Artificial
  Antisense siRNA sequence
aacgcuuguccuucugugg

SEQ ID NO: 809 - DNA, 19 bp, Artificial
  Antisense siRNA sequence
acgcuuguccuucuguggg

SEQ ID NO: 810 - DNA, 19 bp, Artificial
  Antisense siRNA sequence
cgcuuguccuucugugggu

SEQ ID NO: 811 - DNA, 19 bp, Artificial
  Antisense siRNA sequence
gcuuguccuucuguggguc

SEQ ID NO: 812 - DNA, 19 bp, Artificial
  Antisense siRNA sequence
cuuguccuucugugggucg

SEQ ID NO: 813 - DNA, 19 bp, Artificial
  Antisense siRNA sequence
uuguccuucugugggucgu

SEQ ID NO: 814 - DNA, 19 bp, Artificial
  Antisense siRNA sequence
uguccuucugugggucgua

SEQ ID NO: 815 - DNA, 19 bp, Artificial
  Antisense siRNA sequence
guccuucugugggucguag

SEQ ID NO: 816 - DNA, 19 bp, Artificial
  Antisense siRNA sequence
uccuucugugggucguagu

SEQ ID NO: 817 - DNA, 19 bp, Artificial
  Antisense siRNA sequence
ccuucugugggucguaguu

SEQ ID NO: 818 - DNA, 19 bp, Artificial
  Antisense siRNA sequence
cuucugugggucguaguuu

SEQ ID NO: 819 - DNA, 19 bp, Artificial
  Antisense siRNA sequence
uucugugggucguaguuuu

SEQ ID NO: 820 - DNA, 19 bp, Artificial
  Antisense siRNA sequence
ucugugggucguaguuuuu

SEQ ID NO: 821 - DNA, 19 bp, Artificial
  Antisense siRNA sequence
cugugggucguaguuuuuc

SEQ ID NO: 822 - DNA, 19 bp, Artificial
  Antisense siRNA sequence
ugugggucguaguuuuuca

SEQ ID NO: 823 - DNA, 19 bp, Artificial
  Antisense siRNA sequence
gugggucguaguuuuucag

SEQ ID NO: 824 - DNA, 19 bp, Artificial
  Antisense siRNA sequence
ugggucguaguuuuucaga

SEQ ID NO: 825 - DNA, 19 bp, Artificial
  Antisense siRNA sequence
gggucguaguuuuucagag

SEQ ID NO: 826 - DNA, 19 bp, Artificial
  Antisense siRNA sequence
ggucguaguuuuucagagc

SEQ ID NO: 827 - DNA, 19 bp, Artificial
  Antisense siRNA sequence
gucguaguuuuucagagca

SEQ ID NO: 828 - DNA, 19 bp, Artificial
  Antisense siRNA sequence
ucguaguuuuucagagcaa

SEQ ID NO: 829 - DNA, 19 bp, Artificial
  Antisense siRNA sequence
cguaguuuuucagagcaau

SEQ ID NO: 830 - DNA, 19 bp, Artificial
  Antisense siRNA sequence
guaguuuuucagagcaauu

SEQ ID NO: 831 - DNA, 19 bp, Artificial
  Antisense siRNA sequence
uaguuuuucagagcaauuu

SEQ ID NO: 832 - DNA, 19 bp, Artificial
  Antisense siRNA sequence
aguuuuucagagcaauuug

SEQ ID NO: 833 - DNA, 19 bp, Artificial
  Antisense siRNA sequence
guuuuucagagcaauuugc

SEQ ID NO: 834 - DNA, 19 bp, Artificial
  Antisense siRNA sequence
uuuuucagagcaauuugca

SEQ ID NO: 835 - DNA, 19 bp, Artificial
  Antisense siRNA sequence
uuuucagagcaauuugcaa

SEQ ID NO: 836 - DNA, 19 bp, Artificial
  Antisense siRNA sequence
uuucagagcaauuugcaau

SEQ ID NO: 837 - DNA, 19 bp, Artificial
  Antisense siRNA sequence
uucagagcaauuugcaauu

SEQ ID NO: 838 - DNA, 19 bp, Artificial
  Antisense siRNA sequence
ucagagcaauuugcaauuc

SEQ ID NO: 839 - DNA, 19 bp, Artificial
  Antisense siRNA sequence
cagagcaauuugcaauuca

SEQ ID NO: 840 - DNA, 19 bp, Artificial
  Antisense siRNA sequence
agagcaauuugcaauucaa

SEQ ID NO: 841 - DNA, 19 bp, Artificial
  Antisense siRNA sequence
gagcaauuugcaauucaau

SEQ ID NO: 842 - DNA, 19 bp, Artificial
  Antisense siRNA sequence
agcaauuugcaauucaauc

SEQ ID NO: 843 - DNA, 19 bp, Artificial
  Antisense siRNA sequence
gcaauuugcaauucaaucg

SEQ ID NO: 844 - DNA, 19 bp, Artificial
  Antisense siRNA sequence
caauuugcaauucaaucgu

SEQ ID NO: 845 - DNA, 19 bp, Artificial
  Antisense siRNA sequence
aauuugcaauucaaucguu

SEQ ID NO: 846 - DNA, 19 bp, Artificial
  Antisense siRNA sequence
auuugcaauucaaucguuu

SEQ ID NO: 847 - DNA, 19 bp, Artificial
  Antisense siRNA sequence
uuugcaauucaaucguuuc

SEQ ID NO: 848 - DNA, 19 bp, Artificial
  Antisense siRNA sequence
uugcaauucaaucguuucu

SEQ ID NO: 849 - DNA, 19 bp, Artificial
  Antisense siRNA sequence
ugcaauucaaucguuucuc

SEQ ID NO: 850 - DNA, 19 bp, Artificial
  Antisense siRNA sequence
gcaauucaaucguuucucg

SEQ ID NO: 851 - DNA, 19 bp, Artificial
  Antisense siRNA sequence
caauucaaucguuucucgg

SEQ ID NO: 852 - DNA, 19 bp, Artificial
  Antisense siRNA sequence
aauucaaucguuucucgga

SEQ ID NO: 853 - DNA, 19 bp, Artificial
  Antisense siRNA sequence
auucaaucguuucucggaa

SEQ ID NO: 854 - DNA, 19 bp, Artificial
  Antisense siRNA sequence
uucaaucguuucucggaaa

SEQ ID NO: 855 - DNA, 19 bp, Artificial
  Antisense siRNA sequence
ucaaucguuucucggaaau

SEQ ID NO: 856 - DNA, 19 bp, Artificial
  Antisense siRNA sequence
caaucguuucucggaaauu

SEQ ID NO: 857 - DNA, 19 bp, Artificial
  Antisense siRNA sequence
aaucguuucucggaaauug

SEQ ID NO: 858 - DNA, 19 bp, Artificial
  Antisense siRNA sequence
aucguuucucggaaauugc

SEQ ID NO: 859 - DNA, 19 bp, Artificial
  Antisense siRNA sequence
ucguuucucggaaauugcg

SEQ ID NO: 860 - DNA, 19 bp, Artificial
  Antisense siRNA sequence
cguuucucggaaauugcgc

SEQ ID NO: 861 - DNA, 19 bp, Artificial
  Antisense siRNA sequence
guuucucggaaauugcgcu

SEQ ID NO: 862 - DNA, 19 bp, Artificial
  Antisense siRNA sequence
uuucucggaaauugcgcuu

SEQ ID NO: 863 - DNA, 19 bp, Artificial
  Antisense siRNA sequence
uucucggaaauugcgcuuc

SEQ ID NO: 864 - DNA, 19 bp, Artificial
  Antisense siRNA sequence
ucucggaaauugcgcuucu

SEQ ID NO: 865 - DNA, 19 bp, Artificial
  Antisense siRNA sequence
cucggaaauugcgcuucuu

SEQ ID NO: 866 - DNA, 19 bp, Artificial
  Antisense siRNA sequence
ucggaaauugcgcuucuuc

SEQ ID NO: 867 - DNA, 19 bp, Artificial
  Antisense siRNA sequence
cggaaauugcgcuucuucu

SEQ ID NO: 868 - DNA, 19 bp, Artificial
  Antisense siRNA sequence
ggaaauugcgcuucuucuc

SEQ ID NO: 869 - DNA, 19 bp, Artificial
  Antisense siRNA sequence
gaaauugcgcuucuucucu

SEQ ID NO: 870 - DNA, 19 bp, Artificial
  Antisense siRNA sequence
aaauugcgcuucuucucuu

SEQ ID NO: 871 - DNA, 19 bp, Artificial
  Antisense siRNA sequence
aauugcgcuucuucucuug

SEQ ID NO: 872 - DNA, 19 bp, Artificial
  Antisense siRNA sequence
auugcgcuucuucucuugg

SEQ ID NO: 873 - DNA, 19 bp, Artificial
  Antisense siRNA sequence
uugcgcuucuucucuuggg

SEQ ID NO: 874 - DNA, 19 bp, Artificial
  Antisense siRNA sequence
ugcgcuucuucucuuggga

SEQ ID NO: 875 - DNA, 19 bp, Artificial
  Antisense siRNA sequence
gcgcuucuucucuugggau

SEQ ID NO: 876 - DNA, 19 bp, Artificial
  Antisense siRNA sequence
cgcuucuucucuugggauu

SEQ ID NO: 877 - DNA, 19 bp, Artificial
  Antisense siRNA sequence
gcuucuucucuugggauug

SEQ ID NO: 878 - DNA, 19 bp, Artificial
  Antisense siRNA sequence
cuucuucucuugggauugu

SEQ ID NO: 879 - DNA, 19 bp, Artificial
  Antisense siRNA sequence
uucuucucuugggauuguu

SEQ ID NO: 880 - DNA, 19 bp, Artificial
  Antisense siRNA sequence
ucuucucuugggauuguuu

SEQ ID NO: 881 - DNA, 19 bp, Artificial
  Antisense siRNA sequence
cuucucuugggauuguuuc

SEQ ID NO: 882 - DNA, 19 bp, Artificial
  Antisense siRNA sequence
uucucuugggauuguuuca

SEQ ID NO: 883 - DNA, 19 bp, Artificial
  Antisense siRNA sequence
ucucuugggauuguuucaa

SEQ ID NO: 884 - DNA, 19 bp, Artificial
  Antisense siRNA sequence
cucuugggauuguuucaag

SEQ ID NO: 885 - DNA, 19 bp, Artificial
  Antisense siRNA sequence
ucuugggauuguuucaaga

SEQ ID NO: 886 - DNA, 19 bp, Artificial
  Antisense siRNA sequence
cuugggauuguuucaagau

SEQ ID NO: 887 - DNA, 19 bp, Artificial
  Antisense siRNA sequence
uugggauuguuucaagauu

SEQ ID NO: 888 - DNA, 19 bp, Artificial
  Antisense siRNA sequence
ugggauuguuucaagauuu

SEQ ID NO: 889 - DNA, 19 bp, Artificial
  Antisense siRNA sequence
gggauuguuucaagauuuc

SEQ ID NO: 890 - DNA, 19 bp, Artificial
  Antisense siRNA sequence
ggauuguuucaagauuuca

SEQ ID NO: 891 - DNA, 19 bp, Artificial
  Antisense siRNA sequence
gauuguuucaagauuucag

SEQ ID NO: 892 - DNA, 19 bp, Artificial
  Antisense siRNA sequence
auuguuucaagauuucagc

SEQ ID NO: 893 - DNA, 19 bp, Artificial
  Antisense siRNA sequence
uuguuucaagauuucagcc

SEQ ID NO: 894 - DNA, 19 bp, Artificial
  Antisense siRNA sequence
uguuucaagauuucagcca

SEQ ID NO: 895 - DNA, 19 bp, Artificial
  Antisense siRNA sequence
guuucaagauuucagccac

SEQ ID NO: 896 - DNA, 19 bp, Artificial
  Antisense siRNA sequence
uuucaagauuucagccaca

SEQ ID NO: 897 - DNA, 19 bp, Artificial
  Antisense siRNA sequence
uucaagauuucagccacac

SEQ ID NO: 898 - DNA, 19 bp, Artificial
  Antisense siRNA sequence
ucaagauuucagccacacu

SEQ ID NO: 899 - DNA, 19 bp, Artificial
  Antisense siRNA sequence
caagauuucagccacacuc

SEQ ID NO: 900 - DNA, 19 bp, Artificial
  Antisense siRNA sequence
aagauuucagccacacucu

SEQ ID NO: 901 - DNA, 19 bp, Artificial
  Antisense siRNA sequence
agauuucagccacacucuc

SEQ ID NO: 902 - DNA, 19 bp, Artificial
  Antisense siRNA sequence
gauuucagccacacucucg

SEQ ID NO: 903 - DNA, 19 bp, Artificial
  Antisense siRNA sequence
auuucagccacacucucgu

SEQ ID NO: 904 - DNA, 19 bp, Artificial
  Antisense siRNA sequence
uuucagccacacucucguu

SEQ ID NO: 905 - DNA, 19 bp, Artificial
  Antisense siRNA sequence
uucagccacacucucguuc

SEQ ID NO: 906 - DNA, 19 bp, Artificial
  Antisense siRNA sequence
ucagccacacucucguuca

SEQ ID NO: 907 - DNA, 19 bp, Artificial
  Antisense siRNA sequence
cagccacacucucguucag

SEQ ID NO: 908 - DNA, 19 bp, Artificial
  Antisense siRNA sequence
agccacacucucguucagc

SEQ ID NO: 909 - DNA, 19 bp, Artificial
  Antisense siRNA sequence
gccacacucucguucagcu

SEQ ID NO: 910 - DNA, 19 bp, Artificial
  Antisense siRNA sequence
ccacacucucguucagcug

SEQ ID NO: 911 - DNA, 19 bp, Artificial
  Antisense siRNA sequence
cacacucucguucagcugg

SEQ ID NO: 912 - DNA, 19 bp, Artificial
  Antisense siRNA sequence
acacucucguucagcuggu

SEQ ID NO: 913 - DNA, 19 bp, Artificial
  Antisense siRNA sequence
cacucucguucagcugguc

SEQ ID NO: 914 - DNA, 19 bp, Artificial
  Antisense siRNA sequence
acucucguucagcugguca

SEQ ID NO: 915 - DNA, 19 bp, Artificial
  Antisense siRNA sequence
cucucguucagcuggucac

SEQ ID NO: 916 - DNA, 19 bp, Artificial
  Antisense siRNA sequence
ucucguucagcuggucacg

SEQ ID NO: 917 - DNA, 19 bp, Artificial
  Antisense siRNA sequence
cucguucagcuggucacgu

SEQ ID NO: 918 - DNA, 19 bp, Artificial
  Antisense siRNA sequence
ucguucagcuggucacgug

SEQ ID NO: 919 - DNA, 19 bp, Artificial
  Antisense siRNA sequence
cguucagcuggucacgugu

SEQ ID NO: 920 - DNA, 19 bp, Artificial
  Antisense siRNA sequence
guucagcuggucacgugug

SEQ ID NO: 921 - DNA, 19 bp, Artificial
  Antisense siRNA sequence
uucagcuggucacguguga

SEQ ID NO: 922 - DNA, 19 bp, Artificial
  Antisense siRNA sequence
ucagcuggucacgugugau

SEQ ID NO: 923 - DNA, 19 bp, Artificial
  Antisense siRNA sequence
cagcuggucacgugugauu

SEQ ID NO: 924 - DNA, 19 bp, Artificial
  Antisense siRNA sequence
agcuggucacgugugauuu

SEQ ID NO: 925 - DNA, 19 bp, Artificial
  Antisense siRNA sequence
gcuggucacgugugauuuu

SEQ ID NO: 926 - DNA, 19 bp, Artificial
  Antisense siRNA sequence
cuggucacgugugauuuug

SEQ ID NO: 927 - DNA, 19 bp, Artificial
  Sense siRNA sequence
gaagcgcaauuuccgagaa

SEQ ID NO: 928 - DNA, 19 bp, Artificial
  Sense siRNA sequence
auugcaaauuguuuugaaa

SEQ ID NO: 929 - DNA, 19 bp, Artificial
  Sense siRNA sequence
uugcauccuuggugauuaa

SEQ ID NO: 930 - DNA, 19 bp, Artificial
  Sense siRNA sequence
accugaagaaguugaacaa

SEQ ID NO: 931 - DNA, 481 bp, Heterodera glycines
caaaatcacacgtgaccagctgaacgagagtgtggctgaaatcttgaaacaatcccaagagaagaagcgcaatttccgagaaacgattgaattgcaaattgctctgaaaaactacgacccacagaaggacaagcgtttcagtggaactgttagactgaagcacatccctcgtccgaaaacgaaggtttgcatccttggcgaccaaaaacattgtgacgaggccaatgccaacggaattccatgcatgacagcggacgacctgaagaagttgaacaaggacaagaagctgatctaaaagctcagcaaaagctaccacgctttccttgccttcgaatcactcatcaaacaaatccctcgtattcttggtcccggactgaacaaggctggcaagttcccaagtgtggtgtcacacaacgacatgctgaacgcaaaggtggatgaagtgaaggcgaaccgcaaattcgaaatgaaacaggtgctc

SEQ ID NO: 932 - DNA, 58 bp, Artificial
  shRNA sequence
gaagcgcaatttccgagaatatcaagagtattctcggaaattgcgcttctgttttttt

SEQ ID NO: 933 - DNA, 58 bp, Artificial
  shRNA sequence
acctgaagaagttgaacaatatcaagagtattgttcaacttcttcaggttgttttttt

SEQ ID NO: 934 - DNA, 107 bp, Artificial
  Artificial miRNA sequence
ggatccagctccttgtttctcggaaattgcgcttcttagtctcttggatctcaaatgccactgaacccaagaagcgcaacctccgagaacaacacgggtttgagctc

SEQ ID NO: 935 - DNA, 95 bp, Artificial
  miRNA sequence
agcuccuuguuggagaagcagggcacgugcaagucucuuggaucucaaaugccacugaacccuuugcacgugcuccccuucuccaacacggguuu

SEQ ID NO: 936 - DNA, 19 bp, Artificial
  Mutated siRNA sequence
gaagcgcaaccuccgagaa

SEQ ID NO: 937 - DNA, 95 bp, Artificial
  Artificial miRNA sequence
agcuccuuguuucucggaaauugcgcuucuuagucucuuggaucucaaaugccacugaacccaagaagcgcaaccuccgagaacaacacggguuu